SEQUENCE LISTING (262 sequences)

SEQ ID NO: 1 - DNA, 15 bp, Zea mays
gcggcarccatggcg

SEQ ID NO: 2 - DNA, 15 bp, artificial
  synthetic sequence
gtttattttatgaga

SEQ ID NO: 3 - DNA, 14 bp, Arabidopsis
aaaaraaaatggcg

SEQ ID NO: 4 - DNA, 15 bp, artificial
  synthetic sequence
gggcttcgtatgctc

SEQ ID NO: 5 - DNA, 15 bp, Solanum lycopersicum
ttaacaaaaatggct

SEQ ID NO: 6 - DNA, 15 bp, artificial
  synthetic sequence
  misc_feature (2)..(2): n is a, c, g, or t
cngcgccgtatgcgc

SEQ ID NO: 7 - DNA, 11 bp, Oriza sativa
  misc_feature (2)..(5): B is G or C, D is A or G,  H is A or C,
cbdhbatggcg

SEQ ID NO: 8 - DNA, 100 bp, Zea mays
accgcccggcagggctgctcatctcgtcgacgaccagtggattaatcggcatggcggctctagccacgtcgcagctcgtcgcaacgcgcgccggcctggg

SEQ ID NO: 9 - DNA, 100 bp, Zea mays
ctctctctctctctctccagtctccaccggcagcgctaatcgtaatagccatgggctccaccgccggcgacgtggccgcggtggtggacgaggaggcgtg

SEQ ID NO: 10 - DNA, 100 bp, Zea mays
agccagctccattcatccccattccgtttattcaccgtccgttgcagcgaatgccctcgaggagccagtacgaccgccgcggcagcgatggcgacgagga

SEQ ID NO: 11 - DNA, 100 bp, Zea mays
cccatgatttactttacttgttaagaggaagcacaaactactccaacttcatggcggacttggcgctagttggcttaaggtgggcagcatcgccgattgt

SEQ ID NO: 12 - DNA, 100 bp, Zea mays
cttgttagtttctagaaccttccagacctccagttcccgacctcggcgccatggacctggacccagaggggatcttccgcgacgacagcgatgaagacga

SEQ ID NO: 13 - DNA, 100 bp, Glycine max
ttttctcattttgccaaactaaaatagttgtgttggtagctttggcaaagatgttttcagcaggccataagatcaaagggacagtggtgttgatgcccaa

SEQ ID NO: 14 - DNA, 100 bp, Glycine max
aattcgatcaatcaatccatcttcaattgctttgtttcgatcggaggaaaatggccgatcagttatcgaagggagaggaattcgagaaaaaggctgagaa

SEQ ID NO: 15 - DNA, 100 bp, artificial
  synthetic sequence
accgcccggcagggctgctcatctcgtcgacgaccagtggattaatccccatggcggctctagccacgtcgcagctcgtcgcaacgcgcgccggcctggg

SEQ ID NO: 16 - DNA, 100 bp, artificial
  synthetic sequence
accgcccggcagggctgctcatctcgtcgacgaccagtggattaatccccatgccggctctagccacgtcgcagctcgtcgcaacgcgcgccggcctggg

SEQ ID NO: 17 - DNA, 100 bp, artificial
  synthetic sequence
ctctctctctctctctccagtctccaccggcagcgctaatcgtaatattcatgggctccaccgccggcgacgtggccgcggtggtggacgaggaggcgtg

SEQ ID NO: 18 - DNA, 100 bp, artificial
  synthetic sequence
ctctctctctctctctccagtctccaccggcagcgctaatcgtaatattcatgcgctccaccgccggcgacgtggccgcggtggtggacgaggaggcgtg

SEQ ID NO: 19 - DNA, 100 bp, artificial
  synthetic sequence
agccagctccattcatccccattccgtttattcaccgtccgttgcaggccatgccctcgaggagccagtacgaccgccgcggcagcgatggcgacgagga

SEQ ID NO: 20 - DNA, 100 bp, artificial
  synthetic sequence
agccagctccattcatccccattccgtttattcaccgtccgttgcaggccatggcctcgaggagccagtacgaccgccgcggcagcgatggcgacgagga

SEQ ID NO: 21 - DNA, 100 bp, artificial
  synthetic sequence
cccatgatttactttacttgttaagaggaagcacaaactactccaacttcatgctggacttggcgctagttggcttaaggtgggcagcatcgccgattgt

SEQ ID NO: 22 - DNA, 100 bp, artificial
  synthetic sequence
cccatgatttactttacttgttaagaggaagcacaaactactccaacgccatggcggacttggcgctagttggcttaaggtgggcagcatcgccgattgt

SEQ ID NO: 23 - DNA, 100 bp, artificial
  synthetic sequence
cttgttagtttctagaaccttccagacctccagttcccgacctcggcttcatggacctggacccagaggggatcttccgcgacgacagcgatgaagacga

SEQ ID NO: 24 - DNA, 100 bp, artificial
  synthetic sequence
cttgttagtttctagaaccttccagacctccagttcccgacctcggcttcatgcacctggacccagaggggatcttccgcgacgacagcgatgaagacga

SEQ ID NO: 25 - DNA, 100 bp, artificial
  synthetic sequence
ttttctcattttgccaaactaaaatagttgtgttggtagctttggcaaaaatggcttcagcaggccataagatcaaagggacagtggtgttgatgcccaa

SEQ ID NO: 26 - DNA, 100 bp, artificial
  synthetic sequence
ttttctcattttgccaaactaaaatagttgtgttggtagctttggcaccgatgttttcagcaggccataagatcaaagggacagtggtgttgatgcccaa

SEQ ID NO: 27 - DNA, 100 bp, artificial
  synthetic sequence
aattcgatcaatcaatccatcttcaattgctttgtttcgatcggaaaaaaatggccgatcagttatcgaagggagaggaattcgagaaaaaggctgagaa

SEQ ID NO: 28 - DNA, 100 bp, artificial
  synthetic sequence
aattcgatcaatcaatccatcttcaattgctttgtttcgatcggaggcgtatgcccgatcagttatcgaagggagaggaattcgagaaaaaggctgagaa

SEQ ID NO: 29 - DNA, 23 bp, Zea mays
atcggcatggcggctctagccac

SEQ ID NO: 30 - DNA, 23 bp, Zea mays
cgacgagctgcgacgtggctaga

SEQ ID NO: 31 - DNA, 23 bp, Zea mays
atggcggacttggcgctagttgg

SEQ ID NO: 32 - DNA, 23 bp, Zea mays
agccaactagcgccaagtccgcc

SEQ ID NO: 33 - DNA, 23 bp, Zea mays
acttcatggcggacttggcgcta

SEQ ID NO: 34 - DNA, 23 bp, Zea mays
tggcggacttggcgctagttggc

SEQ ID NO: 35 - DNA, 23 bp, Zea mays
ccatgaagttggagtagtttgtg

SEQ ID NO: 36 - DNA, 23 bp, Zea mays
ccgacctcggcgccatggacctg

SEQ ID NO: 37 - DNA, 23 bp, Zea mays
gttcccgacctcggcgccatgga

SEQ ID NO: 38 - DNA, 23 bp, Zea mays
cgacctcggcgccatggacctgg

SEQ ID NO: 39 - DNA, 23 bp, Zea mays
gacctcggcgccatggacctgga

SEQ ID NO: 40 - DNA, 23 bp, Zea mays
tggcgccgaggtcgggaactgga

SEQ ID NO: 41 - DNA, 23 bp, Zea mays
ggtccatggcgccgaggtcggga

SEQ ID NO: 42 - DNA, 23 bp, Zea mays
tctgggtccaggtccatggcgcc

SEQ ID NO: 43 - DNA, 23 bp, Zea mays
ctctgggtccaggtccatggcgc

SEQ ID NO: 44 - DNA, 23 bp, Zea mays
ttcaccgtccgttgcagcgaatg

SEQ ID NO: 45 - DNA, 23 bp, Zea mays
accgtccgttgcagcgaatgccc

SEQ ID NO: 46 - DNA, 23 bp, Zea mays
cagcgaatgccctcgaggagcca

SEQ ID NO: 47 - DNA, 23 bp, Zea mays
ttcaccgtccgttgcagcgaatg

SEQ ID NO: 48 - DNA, 23 bp, Zea mays
ccgtccgttgcagcgaatgccct

SEQ ID NO: 49 - DNA, 23 bp, Zea mays
ttgcagcgaatgccctcgaggag

SEQ ID NO: 50 - DNA, 20 bp, Zea mays
gtcgccggcggtggagccca

SEQ ID NO: 51 - DNA, 23 bp, Glycine max
gcaaagatgttttcagcaggcca

SEQ ID NO: 52 - DNA, 23 bp, Glycine max
tgttggtagctttggcaaagatg

SEQ ID NO: 53 - DNA, 23 bp, Glycine max
gtagctttggcaaagatgttttc

SEQ ID NO: 54 - DNA, 23 bp, Glycine max
ccaaagctaccaacacaactatt

SEQ ID NO: 55 - DNA, 23 bp, Glycine max
gcaaagatgttttcagcaggcca

SEQ ID NO: 56 - DNA, 23 bp, Glycine max
ccaaagctaccaacacaactatt

SEQ ID NO: 57 - DNA, 23 bp, Glycine max
atcttatggcctgctgaaaacat

SEQ ID NO: 58 - DNA, 23 bp, Glycine max
tttgatcttatggcctgctgaaa

SEQ ID NO: 59 - DNA, 23 bp, Glycine max
gatcggaggaaaatggccgatca

SEQ ID NO: 60 - DNA, 23 bp, Glycine max
tttcgatcggaggaaaatggccg

SEQ ID NO: 61 - DNA, 23 bp, Glycine max
gataactgatcggccattttcct

SEQ ID NO: 62 - DNA, 23 bp, Glycine max
gatcggaggaaaatggccgatca

SEQ ID NO: 63 - DNA, 23 bp, Glycine max
tttcgatcggaggaaaatggccg

SEQ ID NO: 64 - DNA, 23 bp, Glycine max
atcggaggaaaatggccgatcag

SEQ ID NO: 65 - DNA, 23 bp, Glycine max
ataactgatcggccattttcctc

SEQ ID NO: 66 - DNA, 20 bp, Glycine max
ttgtttcgatcggaggaaaa

SEQ ID NO: 67 - DNA, 20 bp, Glycine max
aattgctttgtttcgatcgg

SEQ ID NO: 68 - DNA, 99 bp, artificial
  synthetic sequence
agccagctccattcatccccattccgtttattcaccgtccgttgcaggaaatgccctcgaggagccagtacgaccgccgcggcagcgatggcgacgagg

SEQ ID NO: 69 - DNA, 96 bp, artificial
  synthetic sequence
ttttctcattttgccaaactaaaatagttgtgttggtagctttggcatgttttcagcaggccataagatcaaagggacagtggtgttgatgcccaa

SEQ ID NO: 70 - DNA, 21 bp, Francisella novicida U112
gtaatttctactgttgtagat

SEQ ID NO: 71 - DNA, 76 bp, artificial
  synthetic sequence
gttttagagctagaaatagcaagttaaaataaggctagtccgttatcaacttgaaaaagtggcaccgagtcggtgc

SEQ ID NO: 72 - DNA, 36 bp, artificial
  synthetic sequence
gtcgccggcggtggagcccagttttagagctatgct

SEQ ID NO: 73 - DNA, 89 bp, artificial
  synthetic sequence
gagcatagcaagttaaaataaggctagtccgttatcaacttgaaaaagtggcaccgagtcggtgcaatatccatgggctccaccgccgg

SEQ ID NO: 74 - DNA, 89 bp, artificial
  synthetic sequence
gagcatagcaagttaaaataaggctagtccgttatcaacttgaaaaagtggcaccgagtcggtgcaatatccatgcgctccaccgccgg

SEQ ID NO: 75 - DNA, 37 bp, artificial
  synthetic sequence
gttgtttcgatcggaggaaaagttttagagctatgct

SEQ ID NO: 76 - DNA, 86 bp, artificial
  synthetic sequence
gagcatagcaagttaaaataaggctagtccgttatcaacttgaaaaagtggcaccgagtcggtgcccattttttcctccgatcgaa

SEQ ID NO: 77 - DNA, 45 bp, Zea mays
gtcaagggctggagccgcggtacctggacctgggtctcccctaga

SEQ ID NO: 78 - DNA, 45 bp, artificial
  synthetic sequence
cagttcccgacctcggcactatggacctggacccagaggggatct

SEQ ID NO: 79 - DNA, 45 bp, artificial
  synthetic sequence
gtcaagggctggagccgtggtacctggacctgggtctcccctaga

SEQ ID NO: 80 - DNA, 45 bp, artificial
  synthetic sequence
cagttcccgacctcggcgccatgaacctggacccagaggggatct

SEQ ID NO: 81 - DNA, 45 bp, artificial
  synthetic sequence
gtcaagggctggagccgcggtacttggacctgggtctcccctaga

SEQ ID NO: 82 - DNA, 42 bp, artificial
  synthetic sequence
caattgctttgtttcgatcggagggggatggccgatcagtta

SEQ ID NO: 83 - DNA, 44 bp, artificial
  synthetic sequence
aatcgtaatatccatgggctccaccgccggcgacgtggccgcgg

SEQ ID NO: 84 - DNA, 44 bp, artificial
  synthetic sequence
aatcgtaatatccatgcgctccaccgccggcgacgtggccgcgg

SEQ ID NO: 85 - DNA, 49 bp, artificial
  synthetic sequence
ttcaattgctttgtttcgatcggaggaaaaaatggccgatcagttatcg

SEQ ID NO: 86 - DNA, 14 bp, Zea mays
cccccgccatggcg

SEQ ID NO: 87 - DNA, 15 bp, artificial
  synthetic sequence
cttaccaccatgaac

SEQ ID NO: 88 - DNA, 15 bp, zea mays
gcggcagccatggcg

SEQ ID NO: 89 - DNA, 21 bp, artificial
  synthetic sequence
cttaccaccatggcgtcctcc

SEQ ID NO: 90 - DNA, 21 bp, artificial
  synthetic sequence
cttaccaccatggcttcctcc

SEQ ID NO: 91 - DNA, 18 bp, artificial
  synthetic sequence
cttaccaccatggcggcc

SEQ ID NO: 92 - DNA, 21 bp, artificial
  synthetic sequence
cttaccaccatggcgtccctc

SEQ ID NO: 93 - DNA, 21 bp, artificial
  synthetic sequence
cttaccaccatggcggccctc

SEQ ID NO: 94 - DNA, 21 bp, artificial
  synthetic sequence
gcggcagccatggcgtcctcc

SEQ ID NO: 95 - DNA, 15 bp, artificial
  synthetic sequence
gtgaccgccatggac

SEQ ID NO: 96 - DNA, 21 bp, artificial
  synthetic sequence
gtgaccgccatggcgtcctcc

SEQ ID NO: 97 - DNA, 18 bp, artificial
  synthetic sequence
gtgaccgccatggcggcc

SEQ ID NO: 98 - DNA, 21 bp, artificial
  synthetic sequence
gtgaccgccatggcgtccctc

SEQ ID NO: 99 - DNA, 21 bp, artificial
  synthetic sequence
gtgaccgccatggcggccctc

SEQ ID NO: 100 - DNA, 15 bp, artificial
  synthetic sequence
ggtaccgccatgact

SEQ ID NO: 101 - DNA, 21 bp, artificial
  synthetic sequence
ggtaccgccatggcgtcctcc

SEQ ID NO: 102 - DNA, 18 bp, artificial
  synthetic sequence
ggtaccgccatggcggcc

SEQ ID NO: 103 - DNA, 21 bp, artificial
  synthetic sequence
ggtaccgccatggcgtccctc

SEQ ID NO: 104 - DNA, 21 bp, artificial
  synthetic sequence
ggtaccgccatggcggccctc

SEQ ID NO: 105 - DNA, 15 bp, artificial
  synthetic sequence
gtcgccgccatggcg

SEQ ID NO: 106 - DNA, 17 bp, artificial
  synthetic sequence
gtgcggcagccatggcg

SEQ ID NO: 107 - DNA, 16 bp, artificial
  synthetic sequence
gtcccccgccatggcg

SEQ ID NO: 108 - DNA, 21 bp, artificial
  synthetic sequence
gtcgccgccatggcgtcctcc

SEQ ID NO: 109 - DNA, 18 bp, artificial
  synthetic sequence
gtcgccgccatggcggcc

SEQ ID NO: 110 - DNA, 21 bp, artificial
  synthetic sequence
gtcgccgccatggcgtccctc

SEQ ID NO: 111 - DNA, 21 bp, artificial
  synthetic sequence
gtcgccgccatggcggccctc

SEQ ID NO: 112 - DNA, 23 bp, artificial
  synthetic sequence
gtgcggcagccatggcgtcctcc

SEQ ID NO: 113 - protein, 57 aa, artificial
  synthetic sequence
MNNNIENQCVPYNCLSNPEEVLLDGERISTGNSSIDISLSLVQLLVSNFVPGGGFLV

SEQ ID NO: 114 - protein, 59 aa, artificial
  synthetic sequence
MAANNNIENQCVPYNCLSNPEEVLLDGERISTGNSSIDISLSLVQLLVSNFVPGGGFLV

SEQ ID NO: 115 - protein, 58 aa, artificial
  synthetic sequence
MANNNIENQCVPYNCLSNPEEVLLDGERISTGNSSIDISLSLVQLLVSNFVPGGGFLV

SEQ ID NO: 116 - protein, 58 aa, artificial
  synthetic sequence
MRNNNIENQCVPYNCLSNPEEVLLDGERISTGNSSIDISLSLVQLLVSNFVPGGGFLV

SEQ ID NO: 117 - protein, 60 aa, artificial
  synthetic sequence
MASSNNNIENQCVPYNCLSNPEEVLLDGERISTGNSSIDISLSLVQLLVSNFVPGGGFLV

SEQ ID NO: 118 - protein, 60 aa, artificial
  synthetic sequence
MASLNNNIENQCVPYNCLSNPEEVLLDGERISTGNSSIDISLSLVQLLVSNFVPGGGFLV

SEQ ID NO: 119 - protein, 60 aa, artificial
  synthetic sequence
MAALNNNIENQCVPYNCLSNPEEVLLDGERISTGNSSIDISLSLVQLLVSNFVPGGGFLV

SEQ ID NO: 120 - protein, 57 aa, artificial
  synthetic sequence
MDQTANITDVDAQMDKISDFYFKNELEWKDLPEYPGAYHYIRLDSKKTNMDLDLKAN

SEQ ID NO: 121 - protein, 58 aa, artificial
  synthetic sequence
MADQTANITDVDAQMDKISDFYFKNELEWKDLPEYPGAYHYIRLDSKKTNMDLDLKAN

SEQ ID NO: 122 - protein, 59 aa, artificial
  synthetic sequence
MAADQTANITDVDAQMDKISDFYFKNELEWKDLPEYPGAYHYIRLDSKKTNMDLDLKAN

SEQ ID NO: 123 - protein, 58 aa, artificial
  synthetic sequence
MRDQTANITDVDAQMDKISDFYFKNELEWKDLPEYPGAYHYIRLDSKKTNMDLDLKAN

SEQ ID NO: 124 - protein, 60 aa, artificial
  synthetic sequence
MASSDQTANITDVDAQMDKISDFYFKNELEWKDLPEYPGAYHYIRLDSKKTNMDLDLKAN

SEQ ID NO: 125 - protein, 60 aa, artificial
  synthetic sequence
MASLDQTANITDVDAQMDKISDFYFKNELEWKDLPEYPGAYHYIRLDSKKTNMDLDLKAN

SEQ ID NO: 126 - protein, 60 aa, artificial
  synthetic sequence
MAALDQTANITDVDAQMDKISDFYFKNELEWKDLPEYPGAYHYIRLDSKKTNMDLDLKAN

SEQ ID NO: 127 - protein, 57 aa, artificial
  synthetic sequence
MTNNYTDALQSPEATAISKGAVSAAISISTKVLGLLGVPFAAQIGQLWTFILNALWP

SEQ ID NO: 128 - protein, 60 aa, artificial
  synthetic sequence
MASSTNNYTDALQSPEATAISKGAVSAAISISTKVLGLLGVPFAAQIGQLWTFILNALWP

SEQ ID NO: 129 - protein, 60 aa, artificial
  synthetic sequence
MASLTNNYTDALQSPEATAISKGAVSAAISISTKVLGLLGVPFAAQIGQLWTFILNALWP

SEQ ID NO: 130 - protein, 60 aa, artificial
  synthetic sequence
MAALTNNYTDALQSPEATAISKGAVSAAISISTKVLGLLGVPFAAQIGQLWTFILNALWP

SEQ ID NO: 131 - protein, 58 aa, artificial
  synthetic sequence
MATNNYTDALQSPEATAISKGAVSAAISISTKVLGLLGVPFAAQIGQLWTFILNALWP

SEQ ID NO: 132 - protein, 59 aa, artificial
  synthetic sequence
MAATNNYTDALQSPEATAISKGAVSAAISISTKVLGLLGVPFAAQIGQLWTFILNALWP

SEQ ID NO: 133 - protein, 58 aa, artificial
  synthetic sequence
MRTNNYTDALQSPEATAISKGAVSAAISISTKVLGLLGVPFAAQIGQLWTFILNALWP

SEQ ID NO: 134 - protein, 58 aa, artificial
  synthetic sequence
MAGSKKRRIKQDSKLEKFTNCYSLSKTLRFKAIPVGKTQENIDNKRLLVEDEKRAEDY

SEQ ID NO: 135 - protein, 59 aa, artificial
  synthetic sequence
MAAGSKKRRIKQDSKLEKFTNCYSLSKTLRFKAIPVGKTQENIDNKRLLVEDEKRAEDY

SEQ ID NO: 136 - protein, 58 aa, artificial
  synthetic sequence
MRGSKKRRIKQDSKLEKFTNCYSLSKTLRFKAIPVGKTQENIDNKRLLVEDEKRAEDY

SEQ ID NO: 137 - protein, 60 aa, artificial
  synthetic sequence
MASLGSKKRRIKQDSKLEKFTNCYSLSKTLRFKAIPVGKTQENIDNKRLLVEDEKRAEDY

SEQ ID NO: 138 - protein, 60 aa, artificial
  synthetic sequence
MASSGSKKRRIKQDSKLEKFTNCYSLSKTLRFKAIPVGKTQENIDNKRLLVEDEKRAEDY

SEQ ID NO: 139 - protein, 60 aa, artificial
  synthetic sequence
MAALGSKKRRIKQDSKLEKFTNCYSLSKTLRFKAIPVGKTQENIDNKRLLVEDEKRAEDY

SEQ ID NO: 140 - DNA, 27 bp, Zea mays
tggattaatcggcatggcggctctagc

SEQ ID NO: 141 - DNA, 27 bp, artificial
  synthetic sequence
tggattaatccccatggcggctctagc

SEQ ID NO: 142 - DNA, 27 bp, artificial
  synthetic sequence
tggattaatccccatgccggctctagc

SEQ ID NO: 143 - DNA, 27 bp, Zea mays
aatcgtaatagccatgggctccaccgc

SEQ ID NO: 144 - DNA, 27 bp, artificial
  synthetic sequence
aatcgtaatattcatgggctccaccgc

SEQ ID NO: 145 - DNA, 27 bp, artificial
  synthetic sequence
aatcgtaatattcatgcgctccaccgc

SEQ ID NO: 146 - DNA, 27 bp, Zea mays
tccgttgcagcgaatgccctcgaggag

SEQ ID NO: 147 - DNA, 27 bp, artificial
  synthetic sequence
tccgttgcaggccatgccctcgaggag

SEQ ID NO: 148 - DNA, 27 bp, artificial
  synthetic sequence
tccgttgcaggccatggcctcgaggag

SEQ ID NO: 149 - DNA, 27 bp, Zea mays
ctactccaacttcatggcggacttggc

SEQ ID NO: 150 - DNA, 27 bp, artificial
  synthetic sequence
ctactccaacttcatgctggacttggc

SEQ ID NO: 151 - DNA, 27 bp, artificial
  synthetic sequence
ctactccaacgccatggcggacttggc

SEQ ID NO: 152 - DNA, 27 bp, Zea mays
cgacctcggcgccatggacctggaccc

SEQ ID NO: 153 - DNA, 27 bp, artificial
  synthetic sequence
cgacctcggcttcatggacctggaccc

SEQ ID NO: 154 - DNA, 27 bp, artificial
  synthetic sequence
cgacctcggcttcatgcacctggaccc

SEQ ID NO: 155 - DNA, 29 bp, Glycine max
gtagctttggcaaagatgttttcagcagg

SEQ ID NO: 156 - DNA, 29 bp, artificial
  synthetic sequence
gtagctttggcaaaaatggcttcagcagg

SEQ ID NO: 157 - DNA, 29 bp, artificial
  synthetic sequence
gtagctttggcaccgatgttttcagcagg

SEQ ID NO: 158 - DNA, 29 bp, Glycine max
ttcgatcggaggaaaatggccgatcagtt

SEQ ID NO: 159 - DNA, 29 bp, artificial
  synthetic sequence
ttcgatcggaaaaaaatggccgatcagtt

SEQ ID NO: 160 - DNA, 29 bp, artificial
  synthetic sequence
ttcgatcggaggcgtatgcccgatcagtt

SEQ ID NO: 161 - DNA, 37 bp, Zea mays
tccgtttattcaccgtccgttgcagcgaatgccctcg

SEQ ID NO: 162 - DNA, 36 bp, artificial
  synthetic sequence
tccgtttattcaccgtccgttgcaggaatgccctcg

SEQ ID NO: 163 - DNA, 52 bp, Glycine max
gtgttggtagctttggcaaagatgttttcagcaggccataagatcaaaggga

SEQ ID NO: 164 - DNA, 48 bp, artificial
  synthetic sequence
gtgttggtagctttggcatgttttcagcaggccataagatcaaaggga

SEQ ID NO: 165 - DNA, 45 bp, Zea mays
cagttcccgacctcggcgccatggacctggacccagaggggatct

SEQ ID NO: 166 - DNA, 42 bp, Glycine max
caattgctttgtttcgatcggaggaaaatggccgatcagtta

SEQ ID NO: 167 - DNA, 44 bp, Zea mays
aatcgtaatagccatgggctccaccgccggcgacgtggccgcgg

SEQ ID NO: 168 - DNA, 47 bp, Glycine max
ttcaattgctttgtttcgatcggaggaaaatggccgatcagttatcg

SEQ ID NO: 169 - DNA, 20 bp, Lachnospiraceae bacterium ND2006
gaatttctactgttgtagat

SEQ ID NO: 170 - DNA, 120 bp, Glycine max
ttcttctttattttctcattttgccaaactaaaatagttgtgttggtagctttggcaaagatgttttcagcaggccataagatcaaagggacagtggtgttgatgcccaagaatgagttg

SEQ ID NO: 171 - DNA, 120 bp, Glycine max
ttggtttcgttgttcgaatctgttatctatttccgaaccaataatgcgacgcgaacgttaatggagaaagaaggtgatgcattagtggatcctctctccaactattggggactttccctc

SEQ ID NO: 172 - DNA, 120 bp, Glycine max
gagacttcacagaccaaagtgcagagaacaacaaagttcacaactttaaggaaaattgaaatggcccaagtgagcagagtgcacaatcttgctcaaagcactcaaatttttggccattct

SEQ ID NO: 173 - DNA, 120 bp, Glycine max
aactcacctctcccaaatccgtttcctctttcaaacctttttttttccaccaaatcggcgatggcaacgacgtcggtggtgccggcgaaccggcgtcgcgcggcgaaccccgcggaggag

SEQ ID NO: 174 - DNA, 120 bp, Glycine max
tcttctctagggtttcaatttcaccatgtttccctttctcaaattagggttccggcgagcatggccgacggtccggcgagtccaggcggcggtagccacgagagcggcgagcacagccct

SEQ ID NO: 175 - DNA, 120 bp, Glycine max
gcgtaacatatcattggagtttccaatttagattaacatagtgtgttgattttgggattgatgcttgcggtgtcacctttgaggagcaatatcaaagatgaaaagaagcaaggagagatg

SEQ ID NO: 176 - DNA, 120 bp, Glycine max
cttttgtctttgtcttcatcttcggtgaatgatcactgatcactcatcatcatcatcatcatgggggtgttacaggagaagctctcttccttctacaagagccggtggctagtgttcgtg

SEQ ID NO: 177 - DNA, 120 bp, Glycine max
ctgcacccctccctccttgtttgtatcatatcaggcagctcagaaggcaaagatagatccatgtccaccaccgcaactacaacctctgaaggtttttttttaaatttcattccgtgctac

SEQ ID NO: 178 - DNA, 120 bp, Glycine max
gatccgagacagaaaaaggggttagggtttgttcttgtgatcgaagctaagcgaagcaaaatgggtggatcagtgagggacgtgaagtcgaagggcgaggtcgacgaggtggtggcgagt

SEQ ID NO: 179 - DNA, 120 bp, Glycine max
tatgttgtcaacgttctctatttttcttcgcagaaagcgaatcgattccgattcctctcaatggctgcggaacccaaggaatcgccttccaacaaccccggtctccatacaactcctgac

SEQ ID NO: 180 - DNA, 120 bp, Glycine max
tcaaaacatcagaagaatcattttctttttttaatttttgaaattaacgttcaggcgaagatgaagggtaagacttgcgagctttgtgatcaacaagcttctctctattgtccctccgat

SEQ ID NO: 181 - DNA, 120 bp, Glycine max
agttgttgcataacttgcatcgaacaattaatagaaataacagaaaattaaaaaagaaatatggttgctttcactgagaagcaagatgctttggtgagtagctcattcgaagcattcaag

SEQ ID NO: 182 - DNA, 120 bp, Glycine max
tgcgtaatattgtgaaatttatcttgcagggttaagcggtgagatttgcgaatctgagaaatggcggattcttctcgggaggagaacgtttacatggcgaaattggcggagcaggccgag

SEQ ID NO: 183 - DNA, 120 bp, Glycine max
tatttgacttccctttcccctgataaaaaaaacacacactttcttgccattttccaagccatgtcaacctcaagaacctcagatgcttccctcaaaggatatgagtccaaccaaacccaa

SEQ ID NO: 184 - DNA, 23 bp, Glycine max
gcaaagatgttttcagcaggcca

SEQ ID NO: 185 - DNA, 23 bp, Glycine max
ccaaagctaccaacacaactatt

SEQ ID NO: 186 - DNA, 23 bp, Glycine max
gtagctttggcaaagatgttttc

SEQ ID NO: 187 - DNA, 23 bp, Glycine max
tgttggtagctttggcaaagatg

SEQ ID NO: 188 - DNA, 23 bp, Glycine max
gcaaagatgttttcagcaggcca

SEQ ID NO: 189 - DNA, 23 bp, Glycine max
ccaaagctaccaacacaactatt

SEQ ID NO: 190 - DNA, 23 bp, Glycine max
atcttatggcctgctgaaaacat

SEQ ID NO: 191 - DNA, 23 bp, Glycine max
tttgatcttatggcctgctgaaa

SEQ ID NO: 192 - DNA, 23 bp, Glycine max
tccattaacgttcgcgtcgcatt

SEQ ID NO: 193 - DNA, 23 bp, Glycine max
cgaaccaataatgcgacgcgaac

SEQ ID NO: 194 - DNA, 23 bp, Glycine max
tttctccattaacgttcgcgtcg

SEQ ID NO: 195 - DNA, 23 bp, Glycine max
acgttcgcgtcgcattattggtt

SEQ ID NO: 196 - DNA, 23 bp, Glycine max
tctatttccgaaccaataatgcg

SEQ ID NO: 197 - DNA, 23 bp, Glycine max
tccattaacgttcgcgtcgcatt

SEQ ID NO: 198 - DNA, 23 bp, Glycine max
cgaaccaataatgcgacgcgaac

SEQ ID NO: 199 - DNA, 23 bp, Glycine max
ctaatgcatcaccttctttctcc

SEQ ID NO: 200 - DNA, 23 bp, Glycine max
ttaacgttcgcgtcgcattattg

SEQ ID NO: 201 - DNA, 23 bp, Glycine max
aaccaataatgcgacgcgaacgt

SEQ ID NO: 202 - DNA, 23 bp, Glycine max
aggaaaattgaaatggcccaagt

SEQ ID NO: 203 - DNA, 23 bp, Glycine max
agcaagattgtgcactctgctca

SEQ ID NO: 204 - DNA, 23 bp, Glycine max
acaactttaaggaaaattgaaat

SEQ ID NO: 205 - DNA, 23 bp, Glycine max
ggccatttcaattttccttaaag

SEQ ID NO: 206 - DNA, 23 bp, Glycine max
tgcactctgctcacttgggccat

SEQ ID NO: 207 - DNA, 23 bp, Glycine max
aggaaaattgaaatggcccaagt

SEQ ID NO: 208 - DNA, 23 bp, Glycine max
agcaagattgtgcactctgctca

SEQ ID NO: 209 - DNA, 23 bp, Glycine max
caactttaaggaaaattgaaatg

SEQ ID NO: 210 - DNA, 23 bp, Glycine max
cgattcctctcaatggctgcgga

SEQ ID NO: 211 - DNA, 23 bp, Glycine max
ctctcaatggctgcggaacccaa

SEQ ID NO: 212 - DNA, 23 bp, Glycine max
cgcagccattgagaggaatcgga

SEQ ID NO: 213 - DNA, 23 bp, Glycine max
cttgggttccgcagccattgaga

SEQ ID NO: 214 - DNA, 23 bp, Glycine max
gattcctctcaatggctgcggaa

SEQ ID NO: 215 - DNA, 23 bp, Glycine max
tctcaatggctgcggaacccaag

SEQ ID NO: 216 - DNA, 23 bp, Glycine max
gcagccattgagaggaatcggaa

SEQ ID NO: 217 - DNA, 23 bp, Glycine max
ttgggttccgcagccattgagag

SEQ ID NO: 218 - DNA, 23 bp, Glycine max
cctttctcaaattagggttccgg

SEQ ID NO: 219 - DNA, 23 bp, Glycine max
cggcgagcatggccgacggtccg

SEQ ID NO: 220 - DNA, 23 bp, Glycine max
ctttctcaaattagggttccggc

SEQ ID NO: 221 - DNA, 23 bp, Glycine max
ggcgagcatggccgacggtccgg

SEQ ID NO: 222 - DNA, 23 bp, Glycine max
tcagtgaaagcaaccatatttct

SEQ ID NO: 223 - DNA, 23 bp, Glycine max
acgtccctcactgatccacccat

SEQ ID NO: 224 - DNA, 23 bp, Glycine max
cgtccctcactgatccacccatt

SEQ ID NO: 225 - DNA, 23 bp, Glycine max
aggcgaagatgaagggtaagact

SEQ ID NO: 226 - DNA, 23 bp, Glycine max
ggcgaagatgaagggtaagactt

SEQ ID NO: 227 - DNA, 23 bp, Glycine max
ttgccattttccaagccatgtca

SEQ ID NO: 228 - DNA, 23 bp, Glycine max
ttgaggttgacatggcttggaaa

SEQ ID NO: 229 - DNA, 23 bp, Glycine max
tgccattttccaagccatgtcaa

SEQ ID NO: 230 - DNA, 23 bp, Glycine max
tgaggttgacatggcttggaaaa

SEQ ID NO: 231 - DNA, 23 bp, Glycine max
tcctgtaacacccccatgatgat

SEQ ID NO: 232 - DNA, 23 bp, Glycine max
cgaatctgagaaatggcggattc

SEQ ID NO: 233 - DNA, 23 bp, Glycine max
cgaatctgagaaatggcggattc

SEQ ID NO: 234 - DNA, 23 bp, Glycine max
tagttgcggtggtggacatggat

SEQ ID NO: 235 - DNA, 23 bp, Glycine max
aaacctttttttttccaccaaat

SEQ ID NO: 236 - DNA, 23 bp, Glycine max
caccaaatcggcgatggcaacga

SEQ ID NO: 237 - DNA, 23 bp, Glycine max
aaacctttttttttccaccaaat

SEQ ID NO: 238 - DNA, 23 bp, Glycine max
caccaaatcggcgatggcaacga

SEQ ID NO: 239 - DNA, 23 bp, Glycine max
gattaacatagtgtgttgatttt

SEQ ID NO: 240 - DNA, 23 bp, Glycine max
ggattgatgcttgcggtgtcacc

SEQ ID NO: 241 - DNA, 23 bp, Glycine max
gattaacatagtgtgttgatttt

SEQ ID NO: 242 - DNA, 23 bp, Glycine max
ggattgatgcttgcggtgtcacc

SEQ ID NO: 243 - DNA, 70 bp, Artificial Sequence
  Synthetic sequence
agaacaacaaagttcacaactttaaggaaaagaaaaatggcccaagtgagcagagtgcacaatcttgctc

SEQ ID NO: 244 - DNA, 31 bp, Glycine max
ggtagctttggcaaagatgttttcagcaggc

SEQ ID NO: 245 - DNA, 31 bp, Glycine max
tgcgacgcgaacgttaatggagaaagaaggt

SEQ ID NO: 246 - DNA, 31 bp, Glycine max
tttaaggaaaattgaaatggcccaagtgagc

SEQ ID NO: 247 - DNA, 31 bp, Glycine max
ttccaccaaatcggcgatggcaacgacgtcg

SEQ ID NO: 248 - DNA, 31 bp, Glycine max
tagggttccggcgagcatggccgacggtccg

SEQ ID NO: 249 - DNA, 31 bp, Glycine max
gttgattttgggattgatgcttgcggtgtca

SEQ ID NO: 250 - DNA, 31 bp, Glycine max
catcatcatcatcatcatgggggtgttacag

SEQ ID NO: 251 - DNA, 31 bp, Glycine max
aggcaaagatagatccatgtccaccaccgca

SEQ ID NO: 252 - DNA, 31 bp, Glycine max
agctaagcgaagcaaaatgggtggatcagtg

SEQ ID NO: 253 - DNA, 31 bp, Glycine max
attccgattcctctcaatggctgcggaaccc

SEQ ID NO: 254 - DNA, 31 bp, Glycine max
taacgttcaggcgaagatgaagggtaagact

SEQ ID NO: 255 - DNA, 31 bp, Glycine max
aaattaaaaaagaaatatggttgctttcact

SEQ ID NO: 256 - DNA, 31 bp, Glycine max
tttgcgaatctgagaaatggcggattcttct

SEQ ID NO: 257 - DNA, 31 bp, Glycine max
tgccattttccaagccatgtcaacctcaaga

SEQ ID NO: 258 - DNA, 87 bp, Glycine max
aagtgcagagaacaacaaagttcacaactttaaggaaaattgaaatggcccaagtgagcagagtgcacaatcttgctcaaagcactc

SEQ ID NO: 259 - DNA, 87 bp, Artificial Sequence
  Synthetic sequence
aagtgcagagaacaacaaagttcacaactttaaggaaaagaaaaatggcccaagtgagcagagtgcacaatcttgctcaaagcactc

SEQ ID NO: 260 - DNA, 87 bp, Artificial Sequence
  Synthetic sequence
attgaaatggcacaacaaagttcacaactttaaggaaaagaaaaatggcccaagtgagcagagtgcacaatcttgctcaaagcactc

SEQ ID NO: 261 - protein, 1228 aa, Lachnospiraceae bacterium ND2006
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 262 - protein, 1300 aa, Francisella novicida U112
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

(19) United States
(12) Patent Application Publication
(10) Pub. No.: US 2022/0403401 A1
(43) Pub. Date: Dec. 22, 2022

(54) METHODS AND COMPOSITIONS FOR ALTERING PROTEIN ACCUMULATION

(71) Applicant: MONSANTO TECHNOLOGY LLC, St. Louis, MO (US)

(72) Inventors: Keith DECKER, Wildwood, MO (US); Bryan GONTAREK, Ballwin, MO (US); Natalia IVLEVA, Webster Groves, MO (US); Hong LI, St. Louis, MO (US); Matthew MARENGO, Wildwood, MO (US); Ervin NAGY, Lake Saint Louis, MO (US); Brent O'Brien, St. Charles, MO (US); Qungang QI, Wildwood, MO (US); Graziana TARAMINO, Clayton, MO (US)

(73) Assignee: MONSANTO TECHNOLOGY LLC, St. Louis, MO (US)

(21) Appl. No.: 17/836,783

(22) Filed: Jun. 9, 2022

Related U.S. Application Data
(60) Provisional application No. 63/209,836, filed on Jun. 11, 2021.

(51) Int. Cl. C12N 15/82

(52) U.S. Cl. CPC C12N 15/8213; C12N 15/8257

(57) ABSTRACT

The invention provides compositions and methods useful for modulating protein expression in eukaryotic cells. The invention also provides transgenic plants, edited plant cells, plant parts, and seeds comprising depleted or optimized Kozak sequences and methods of their use.

### CROSS-REFERENCE TO RELATED APPLICATIONS

This application claims benefit of U.S. Provisional Application No. 63/209,836, which was filed on Jun. 11, 2021. The entire content of this provisional application is incorporated herein by reference

### FIELD

The present disclosure relates to compositions and methods related to the use of genome editing to alter protein expression levels.

### INCORPORATION OF SEQUENCE LISTING

The instant application contains a Sequence Listing which has been submitted electronically in ASCII format and is hereby incorporated by reference in its entirety. Said ASCII copy, created on Jun. 9, 2022, is named P345055US01_SL.txt and is 86,016 bytes in size as measured in Microsoft Windows®.

### BACKGROUND

The Kozak sequence is a nucleic acid motif that functions as the protein translation initiation site in eukaryotic mRNA transcripts. Kozak sequences regulate the specificity and efficiency of the initiation of translation. Kozak sequences also mediate the recruitment and assembly of ribosomes onto a messenger RNA (mRNA) transcript. Kozak sequence are also known to be involved in the recognition of the proper AUG start codon to initiate translation.

The consensus Kozak sequence varies amongst different species, but it is often contained within about 5 to 8 nucleotides upstream and downstream of an AUG start codon. There are several characterized conserved positional effects for nucleotides within a consensus Kozak sequence that can impact overall strength of translation. Relative to the A nucleotide in the AUG start codon (termed the +1 position), if the +4, −1, −2, and −3 positions of a Kozak sequence match the consensus Kozak sequence for the species it is classified as having strong mRNA translation efficiency. If only one of the −3 and +4 positions of a Kozak sequence match the consensus Kozak sequence for the species it is classified as having adequate mRNA translation efficiency. If neither of the −3 and +4 positions of a Kozak sequence match the consensus Kozak sequence for the species it is classified as having weak mRNA translation efficiency.

Here, Applicant provides novel methods and compositions for altering protein expression levels of a target gene without altering the tissue specific, developmental regulation, and environmental regulation of native gene expression.

### BRIEF DESCRIPTION OF DRAWINGS

FIG. 1 comprises panels (A) and (B). (A) Consensus sequence (top panel) and Sequence logo (bottom panel) of Kozak from analysis of 99 high RNA, high ribosomal protection maize genes. (B) Consensus sequence (top panel) and Sequence logo (bottom panel) of Kozak from analysis of 99 high RNA, high ribosomal protection Arabidopsis genes. Numbers below the consensus sequence denote position of nucleotides relative to the start codon “ATG” where the “A” nucleotide of the start codon is delineated as +1.

FIG. 2. Schematic illustrating the positions (arrows) of conserved Kozak sequence features relative to the Maize consensus sequence. “R” means Adenine (A) or Guanine (G). Numbers below the consensus sequence denote position of nucleotides relative to the start codon “ATG” where the “A” nucleotide of the start codon is delineated as +1.

FIG. 3. Schematic illustrating the positions (arrows) of conserved Kozak sequence features relative to the Dicot conserved Kozak consensus sequence. “R” means Adenine (A) or Guanine (G). Numbers below the consensus sequence denote position of nucleotides relative to the start codon “ATG” where the “A” nucleotide of the start codon is delineated as +1.

FIG. 4. Schematic of genomic sequence of regions around the Kozak sequences of five Zea mays (Zm) and two Glycine max (Gm) genes. The core Kozak consensus sequence comprising positions −3 to +4 (for Zm) and −4 to +5 (for Gm) are shown in bold. The strength classifications (strong, adequate, weak) are indicated. Under each wild type (WT) Kozak sequence, two putative edited sequences (Ed) are listed which would covert the WT Kozak sequence to a Kozak with an alternative strength classification. Shaded nucleotides indicate point mutations relative to the WT sequence. Bent arrows denote start codon.

FIG. 5 comprises panels (A) and (B). Schematic of targeted mutations of Kozak sequences achievable by insertions or deletions at CRISPR target sites. (A) shows conversion of the wild-type (WT) weak Kozak sequence of ZmRad54 to an adequate Kozak sequence by deleting a ‘C’ (shaded) in the −3 position, thus sliding a flanking ‘G’ into the −3 position. (B) conversion of the WT adequate Kozak sequence of the GmLOX gene into a weak Kozak sequence by a 4-bp ‘AAAG’ deletion (shaded). The core Kozak sequence is shown in bold. PAM sites for Fn- or LbCas12a are shown in italics. Arrows indicate Cas12a gRNA target sites. Bent arrows indicate the start codon. Filled triangle indicates deletions.

FIG. 6 comprises panels (A) and (B). Alignments of the native sequence of Kozak containing portions of genes encoding proteins of interest with examples of modified Kozak sequences obtainable using base editing to alter the mRNA translational efficiency. (A) Alignment of the native strong Kozak sequence of ZmKu70 to examples of engineered weak Kozak sequences achievable with cytosine base editing (CBE). Either of the C to T changes (shaded) shown in panels (i) or (ii) would create an adequate Kozak, while both changes would create a weak Kozak sequence. (B) Alignment of the adequate native Kozak sequence of alpha SNAP of soy to examples of engineered weak Kozak sequences achievable by using adenosine base editing (ABE) to turn one or more ‘A’s to ‘G’s (shaded) as indicated. The change can be mediated by either (i) LbCas12a or (ii) LbCas12-RR. Core Kozak sequences are shown in bold. PAM sites are shown in italics. Arrows indicate Cas12a gRNA target sites. Arrowhead indicates start codon. Box represents 8-14 bp region of the target site, known in the art to be most accessible to Cas12a base editors.

FIG. 7 comprises panels (A) and (B). Alignments of the sequence of Kozak containing portions of genes encoding proteins of interest with sequences of PEtracrRNAs useful in prime editing to alter the ribosome-binding properties of Kozak sequences. (A) Two examples of PEtracrRNA designs useful for prime editing to convert the wild type strong Kozak sequence of the ZmBM3 gene of maize (ZmBM3_WT_Strong) to either adequate (ZmBM3_Ed_Adeq) or weak (ZmBM3 Ed Weak) Kozak sequences. Shaded areas are 7-bp addition inserted into the Cas9 nick site by prime editing, which represent a new Kozak sequence. (B) An example of a PEtracrRNA design for prime editing useful to convert the adequate Kozak sequence of alpha SNAP gene of soy (GmaSNAP_WT_Adeq) to a strong Kozak sequence (GmaSNAP_WT_Strong). Shaded areas are a 2-bp addition inserted into the Cas9 nick site by prime editing, which represent a new Kozak sequence. The core Kozak sequence is shown in bold. PAM sites are shown in italics. Arrows indicate Cas9 gRNA target sites. Arrowhead indicates start codon. Lowercase nucleotides in PEtracrRNA indicate nucleotides from Cas9 tracrRNA. Uppercase nucleotides in PEtracrRNA indicate unique 3′ extensions.

FIG. 8 comprises panels (A), (B), (C), and (D). Amino terminal alignments of approximately first 60 amino acids of representatives of (A) Protein of Interest 1, (B) Protein of Interest 2, (C) Protein of Interest 3, and (D) Protein of Interest 4 described in Table 5. N-terminal modifications are indicated by shading. POI 1-1, POI 2-1, POI 3-1 and POI 4-1 are the native/original protein sequences.

FIG. 9 comprises panels (A), (B), (C), and (D). Graphical depictions of protein accumulation of Kozak and N-terminal variants of (A) POI 1, (B) POI 2, (C) POI 3 and (D) POI 4 in protoplasts. Bar heights and error bars represent means±std errors. Different letters within each Protein of Interest graph represent intervals of Kozak/N-terminal modifications with a significantly different protein expression (α=0.05, Tukey familywise error control after type III Analysis of Variance with Satterthwaite's method). Multiple letters indicate overlapping intervals.

FIG. 10 comprises panels (A), (B), (C), and (D). Graphical depictions of normalized RNA accumulation shown in log 2 space for Kozak and N-terminal variants of (A) POI 1, (B) POI 2, (C) POI 3 and (D) POI 4 in protoplasts. Bar heights and error bars represent means±std error. Different letters within each protein of interest graph represent intervals of Kozak/N-terminal modifications with a significantly different protein expression (α=0.05, Tukey familywise error control after type III Analysis of Variance with Satterthwaite's method). Multiple letters indicate overlapping intervals.

FIG. 11 comprises panels (A) and (B). Graphical depictions of protein accumulation measured from Kozak and N-terminal variants of (A) POI 1 and (B) POI 3 in stably transformed F1 maize plants. Different letters within each Protein of Interest graph represent intervals of Kozak/N-terminal modifications with a significantly different protein expression (α=0.05, Tukey familywise error control).

FIG. 12 comprises panels (A) and (B). Graphical depictions of normalized RNA accumulation shown in log 2 space for Kozak and N-terminal variants of (A) POI 1 and (B) POI 3 in stably transformed F1 maize plants. ANOVA 21.94, p=0.0000115. Letters above bars represent different 95% confidence intervals via Tukey's contrasts.

FIG. 13. Alignment of genomic sequence around the Kozak sequences of thirteen Glycine max (Gm) genes. The core Kozak consensus sequence comprising positions −4 to +5 are shown in bold. The mRNA translational efficiency classifications of the native Kozak sequences (strong, adequate, weak) are indicated. Bent arrows denote start codon. Part. Indicates Partial. All sequences are shown in the 5′ to 3′ orientation.

FIG. 14. DNA-based chromosome cutting rates in soy protoplasts across various combinations of CRISPR nuclease and gRNAs targeting sites in LOC 344. See Table 10 for a combination of different CRISPR reagents used for each protoplast treatment. Error bars represent standard deviation.

FIG. 15. RNP-based chromosome cutting rates in soy protoplasts across various combinations of CRISPR nuclease, repair templates and gRNAs targeting TS1 in LOC 344. See Table 11 for a combination of different CRISPR reagents and controls used for each protoplast treatment. Error bars represent standard deviation. * indicates p value of 0.05

FIG. 16. RNP-based, HDR-mediated templated editing rates in soy protoplasts across various combinations of CRISPR nuclease, repair templates and gRNAs targeting TS1 in LOC 344. See Table 11 for a combination of different CRISPR reagents and controls used for each protoplast treatment. Error bars represent standard deviation. * indicates p value of 0.05.

FIG. 17. RNP-based, SDSA-mediated partial templated editing rates in soy protoplasts across various combinations of CRISPR nuclease, repair templates and gRNAs targeting TS1 in LOC 344. See Table 11 for a combination of different CRISPR reagents and controls used for each protoplast treatment. Error bars represent standard deviation. * indicate p value of 0.05.

### SUMMARY

Several embodiments relate to a method of altering protein accumulation in an edited eukaryotic cell, the method comprising editing the Kozak sequence of a nucleic acid molecule encoding the protein at one or more nucleotides of positions −9, −8, −7, −6, −5, −4, −3, −2, −1, +4, and +5 of the Kozak sequence to generate an edited nucleic acid molecule comprising an edited Kozak sequence, wherein the edited eukaryotic cell comprising the edited nucleic acid molecule exhibits a statistically significant alteration of the accumulation of the protein as compared to the accumulation of the protein within a control eukaryotic cell comprising a reference nucleic acid sequence. In some embodiments, the protein accumulation is increased in the edited eukaryotic cell as compared to the control eukaryotic cell. In some embodiments, the protein accumulation is increased by at least 20%. In some embodiments, the protein accumulation is decreased in the edited eukaryotic cell as compared to the control eukaryotic cell. In some embodiments, protein accumulation is decreased by at least 20%. In some embodiments, protein accumulation is decreased by at least 2-fold. In some embodiments, the nucleic acid molecule is an endogenous nucleic acid molecule. In some embodiments, the nucleic acid molecule is a transgenic nucleic acid molecule. In some embodiments, accumulation of mRNA transcribed from the edited nucleic acid molecule in the edited eukaryotic cell is increased as compared to accumulation of mRNA transcribed from the reference sequence in the control eukaryotic cell. In some embodiments, accumulation of mRNA transcribed from the edited nucleic acid molecule in the edited eukaryotic cell is decreased as compared to accumulation of mRNA transcribed from the reference sequence in the control eukaryotic cell. In some embodiments, accumulation of mRNA transcribed from the edited nucleic acid molecule in the edited eukaryotic cell is not statistically significantly different as compared to accumulation of mRNA transcribed from the reference sequence in the control eukaryotic cell. In some embodiments, the eukaryotic cell is selected from the group consisting of a plant cell, a fungal cell, and an animal cell. In some embodiments, the plant cell is selected from the group consisting of a dicot cell and a monocot cell. In some embodiments, the plant cell is selected from the group consisting of a corn cell, a soybean cell, a tomato cell, a rice cell, a canola cell, a pepper cell, a wheat cell, a cucumber cell, an onion cell, an oilseed rape cell, and a cotton cell. In some embodiments, the edited Kozak sequence comprises a sequence selected from the group consisting of SEQ ID NOs: 1-7, 85-89, 95 and 105. In some embodiments, the editing comprises the use of a method selected from the group consisting of template editing, base editing, and prime editing. In some embodiments, the edited Kozak sequence is a depleted Kozak sequence. In some embodiments, the protein comprises one or more N-terminal amino acid modifications. In some embodiments, the protein comprises one or more N-terminal amino acid modifications selected from the group consisting of: Alanine; Arginine; Methionine-Alanine-Serine-Serine wherein Alanine is coded by the codon GCG; Methionine-Alanine-Serine-Serine wherein Alanine is coded by the codon GCT; Methionine-Alanine-Alanine; Methionine-Alanine-Serine-Leucine; and Methionine-Alanine-Alanine-Leucine. In some embodiments, an A or G at the −3 position is edited to a C or T. In some embodiments, a G at the +4 position is edited to an A, C, or T. In some embodiments, a C at the −1 position is edited to an A, G, or T. In some embodiments, a C at the −2 position is edited to an A, G, or T. In some embodiments, an A at the −4 position is edited to a G, C, or T. In some embodiments, an A at the −3 position is edited to a G, C, or T. In some embodiments, an A at the −2 position is edited to a G, C, or T. In some embodiments, an A at the −1 position is edited to a G, C, or T. In some embodiments, a G at the +4 position is edited to an A, C, or T. In some embodiments, a C at the +5 position is edited to an A, G, or T.

Several embodiments relate to a method of generating an edited plant, the method comprising: (a) providing an editing enzyme, or a nucleic acid molecule encoding the editing enzyme, to a plant cell; (b) generating an edit in a Kozak sequence of a nucleic acid molecule encoding a protein in the plant cell to generate an edited Kozak sequence, wherein the edit comprises editing the Kozak sequence in one or more nucleotide positions of the Kozak sequence selected from the group consisting of positions −9, −8, −7, −6, −5, −4, −3, −2, −1, +4, and +5; and (c) regenerating an edited plant from the plant cell, wherein the edited plant comprises the edited Kozak sequence, and wherein accumulation of the protein is altered in the edited plant as compared to a control plant when grown under comparable conditions. In some embodiments, the editing enzyme is selected from the group consisting of a Cas9 nuclease, a Cas12a nuclease, a cytosine base editor, an adenine base editor, a Cas9 nickase, and a Cas12a nickase. In some embodiments, the editing enzyme further comprises an engineered reverse transcriptase. In some embodiments, the method further comprises the use of a guide RNA (gRNA), or a nucleic acid molecule encoding the gRNA.

In some embodiments, the gRNA is a single-gRNA (sgRNA). In some embodiments, the gRNA is a split gRNA. In some embodiments, the editing enzyme and the gRNA are provided as a ribonucleoprotein complex. In some embodiments, the providing comprises a method selected from: Agrobacterium-mediated transformation, particle bombardment, and carbon nanoparticle delivery. In some embodiments, accumulation of the protein is increased in the edited plant as compared to the control plant. In some embodiments, accumulation of the protein is increased at least 20%. In some embodiments, accumulation of the protein is decreased in the edited plant as compared to the control plant. In some embodiments, accumulation of the protein is decreased at least 20%. In some embodiments, the plant cell is selected from the group consisting of a corn cell, a soybean cell, a tomato cell, a rice cell, a canola cell, a pepper cell, a wheat cell, a cucumber cell, an onion cell, an oilseed rape cell, and a cotton cell. In some embodiments, the plant cell is a protoplast cell or a callus cell. In some embodiments, the nucleic acid molecule is an endogenous nucleic acid molecule. In some embodiments, the nucleic acid molecule is a transgenic nucleic acid molecule. In some embodiments, the edited Kozak sequence comprises a sequence selected from the group consisting of SEQ ID NOs: 1-7, 85-89, 95 and 105. In some embodiments, the method further comprises generating an edit resulting in one or more N-terminal amino acid modifications of the protein. In some embodiments, the one or more N-terminal amino acid modifications introduces an N-terminal sequence selected from the group consisting of: Methionine-Alanine-Serine-Serine wherein Alanine is coded by the codon GCG; Methionine-Alanine-Serine-Serine wherein Alanine is coded by the codon GCT; Methionine-Alanine-Alanine; Methionine-Alanine-Serine-Leucine; and Methionine-Alanine-Alanine-Leucine. In some embodiments, an A or G at the −3 position is edited to a C or T. In some embodiments, a G at the +4 position is edited to an A, C, or T. In some embodiments, a C at the −1 position is edited to an A, G, or T. In some embodiments, a C at the −2 position is edited to an A, G, or T. In some embodiments, an A at the −4 position is edited to a G, C, or T. In some embodiments, an A at the −3 position is edited to a G, C, or T. In some embodiments, an A at the −2 position is edited to a G, C, or T. In some embodiments, an A at the −1 position is edited to a G, C, or T. In some embodiments, a G at the +4 position is edited to an A, C, or T. In some embodiments, a C at the +5 position is edited to an A, G, or T.

Several embodiments relate to a prime editing guide RNA (pegRNA) sequence, wherein the pegRNA sequence is capable of directing a prime editor (PE) to a Kozak sequence of a nucleic acid molecule, and wherein the pegRNA comprises a template sequence to edit the Kozak sequence at one or more positions selected from the group consisting of positions −9, −8, −7, −6, −5, −4, −3, −2, −1, +4, and +5 as compared to a reference Kozak sequence. In some embodiments, the pegRNA is a split pegRNA. Several embodiments relate to a DNA molecule encoding pegRNA sequence, wherein the pegRNA sequence is capable of directing a prime editor (PE) to a Kozak sequence of a nucleic acid molecule, and wherein the pegRNA comprises a template sequence to edit the Kozak sequence at one or more positions selected from the group consisting of positions −9, −8, −7, −6, −5, −4, −3, −2, −1, +4, and +5 as compared to a reference Kozak sequence. In some embodiments, the pegRNA is a split pegRNA. In some embodiments, the split pegRNA comprises a prime editing tracrRNA (petracrRNA) and a crRNA. In some embodiments, the template sequence comprises a strong Kozak sequence. In some embodiments, the strong Kozak sequence is selected from the group consisting of SEQ ID NOs: 1, 3, 5, 7, 86, 95 and 105. In some embodiments, the template sequence comprises an adequate Kozak sequence. In some embodiments, the template sequence comprises a weak Kozak sequence. In some embodiments, the template sequence comprises a depleted Kozak sequence. In some embodiments, the depleted Kozak sequence is selected from the group consisting of SEQ ID NOs: 2, 4, and 6. In some embodiments, the pegRNA is part of a ribonucleoprotein complex. In some embodiments, the ribonucleoprotein complex comprises either (a) a Cas9 nickase or (b) a Cas12a nickase; and (c) an engineered reverse transcriptase.

Several embodiments relate to an edited eukaryotic cell comprising a recombinant Kozak sequence within a nucleic acid molecule encoding a target protein, wherein the recombinant Kozak sequence comprises one or more mutations as compared to a reference sequence in nucleotides at one or more positions independently selected from the group consisting of positions −9, −8, −7, −6, −5, −4, −3, −2, −1, +4, and +5, wherein the edited eukaryotic cell exhibits altered accumulation of the target protein compared to a control eukaryotic cell. In some embodiments, the edited eukaryotic cell is an edited plant cell. In some embodiments, the plant cell is selected from the group consisting of a corn cell, a soybean cell, a tomato cell, a rice cell, a canola cell, a pepper cell, a wheat cell, a cucumber cell, an onion cell, an oilseed rape cell, and a cotton cell. In some embodiments, the recombinant Kozak sequence comprises one or more of an A or G at the −3 position; a G at the +4 position; a C at the −1 position; and a C at the −2 position. In some embodiments, the recombinant Kozak sequence comprises an C or T at the −3 position and an A, C, or T at the +4 position. In some embodiments, the recombinant Kozak sequence comprises one or more of a C or T at the −3 position; an A, C or T at the +4 position; an A, G or T at the −1 position; and an A, G or T at the −2 position. In some embodiments, the recombinant Kozak sequence comprises one or more of an A at the −4 position; an A at the −3 position; an A at the −2 position; an A at the −1 position; a G at the +4 position; and a C at the +5 position. In some embodiments, the recombinant Kozak sequence comprises one or more of a C, T, or G at the −4 position; a C, T, or G at the −3 position; a C, T, or G at the −2 position; a C, T, or G at the −1 position; an A, C or T at the +4 position; and an A, G or T at the +5 position. In some embodiments, the recombinant Kozak sequence comprises: (a) at least two A's between positions −4 to −1; or (b) one A between positions −4 and −1 and a G at position +4. In some embodiments, the recombinant Kozak sequence comprises less than two A's between positions −4 and −1 and no G at position +4. In some embodiments, the recombinant Kozak sequence comprises a sequence selected from the group consisting of SEQ ID NOs: 2, 4, and 6. In some embodiments, the recombinant Kozak sequence comprises a sequence selected from the group consisting of SEQ ID NOs: 1, 3, 5, 7, and 86, 95 and 105.

Several embodiments relate to a recombinant DNA molecule comprising a plant expressible promoter operably linked to a heterologous nucleic acid sequence encoding a protein, wherein the nucleic acid sequence comprises a sequence selected from the group consisting of: a) a sequence with at least 90 percent sequence identity to any of SEQ ID NOs: 1-7, 85-89, 95 and 105; and b) a sequence comprising any of SEQ ID NOs: 1-7, 85-89, 95 and 105. In some embodiments, the sequence has at least 95 percent sequence identity to the DNA sequence of any of SEQ ID NOs: 1-7, 85-89, 95 and 105. In some embodiments, the protein confers herbicide tolerance in plants. In some embodiments, the protein confers pest resistance in plants. Several embodiments relate to a transgenic plant cell comprising the recombinant DNA molecule comprising a plant expressible promoter operably linked to a heterologous nucleic acid sequence encoding a protein, wherein the nucleic acid sequence comprises a sequence selected from the group consisting of: a) a sequence with at least 90 percent sequence identity to any of SEQ ID NOs: 1-7, 85-89, 95 and 105; and b) a sequence comprising any of SEQ ID NOs: 1-7, 85-89, 95 and 105. In some embodiments, the transgenic plant cell is a monocotyledonous plant cell. In some embodiments, transgenic plant cell is a dicotyledonous plant cell. Several embodiments relate to a transgenic seed, wherein the seed comprises the recombinant DNA molecule comprising a plant expressible promoter operably linked to a heterologous nucleic acid sequence encoding a protein, wherein the nucleic acid sequence comprises a sequence selected from the group consisting of: a) a sequence with at least 90 percent sequence identity to any of SEQ ID NOs: 1-7, 85-89, 95 and 105; and b) a sequence comprising any of SEQ ID NOs: 1-7, 85-89, 95 and 105.

### DETAILED DESCRIPTION

Unless defined otherwise, all technical and scientific terms used have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York). The inventors do not intend to be limited to a mechanism or mode of action. Reference thereto is provided for illustrative purposes only.

The practice of this disclosure includes, unless otherwise indicated, conventional techniques of biochemistry, chemistry, molecular biology, microbiology, cell biology, plant biology, genomics, biotechnology, and genetics, which are within the skill of the art. See, for example, Green and Sambrook, Molecular Cloning: A Laboratory Manual, 4th edition (2012); Current Protocols In Molecular Biology (F. M. Ausubel, et al. eds., (1987)); Plant Breeding Methodology (N. F. Jensen, Wiley-Interscience (1988)); the series Methods In Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Animal Cell Culture (R. I. Freshney, ed. (1987)); Recombinant Protein Purification: Principles And Methods, 18-1142-75, GE Healthcare Life Sciences; C. N. Stewart, A. Touraev, V. Citovsky, T. Tzfira eds. (2011) Plant Transformation Technologies (Wiley-Blackwell); and R. H. Smith (2013) Plant Tissue Culture: Techniques and Experiments (Academic Press, Inc.).

Any references cited herein, including, e.g., all patents, published patent applications, and non-patent publications, are incorporated herein by reference in their entirety.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc.

As used herein, terms in the singular and the singular forms “a,” “an,” and “the,” for example, include plural referents unless the content clearly dictates otherwise.

Any composition, nucleic acid molecule, polypeptide, cell, plant, etc. provided herein is specifically envisioned for use with any method provided herein.

“Percent identity” or “% identity” means the extent to which two optimally aligned DNA or protein segments are invariant throughout a window of alignment of components, for example nucleotide sequence or amino acid sequence. An “identity fraction” for aligned segments of a test sequence and a reference sequence is the number of identical components that are shared by sequences of the two aligned segments divided by the total number of sequence components in the reference segment over a window of alignment which is the smaller of the full test sequence or the full reference sequence.

“Plant” refers to a whole plant any part thereof, or a cell or tissue culture derived from a plant, comprising any of: whole plants, plant components, or organs (e.g., leaves, stems, roots, etc.), plant tissues, seeds, plant cells, and/or progeny of the same. A plant cell is a biological cell of a plant, taken from a plant or derived through culture from a cell taken from a plant.

“Promoter” as used herein refers to a nucleic acid sequence located upstream or 5′ to a translational start codon of an open reading frame (or protein-coding region) of a gene and that is involved in recognition and binding of RNA polymerase I, II, or III and other proteins (trans-acting transcription factors) to initiate transcription. A “plant promoter” is a native or non-native promoter that is functional in plant cells. Constitutive promoters are functional in most or all tissues of a plant throughout plant development. Tissue-, organ- or cell-specific promoters are expressed only or predominantly in a particular tissue, organ, or cell type, respectively. Rather than being expressed “specifically” in a given tissue, plant part, or cell type, a promoter may display “enhanced” expression, a higher level of expression, in one cell type, tissue, or plant part of the plant compared to other parts of the plant. Temporally regulated promoters are functional only or predominantly during certain periods of plant development or at certain times of day, as in the case of genes associated with circadian rhythm, for example. Inducible promoters selectively express an operably linked DNA sequence in response to the presence of an endogenous or exogenous stimulus, for example by chemical compounds (chemical inducers) or in response to environmental, hormonal, chemical, and/or developmental signals.

“Recombinant” in reference to a nucleic acid or polypeptide indicates that the material (for example, a recombinant nucleic acid, gene, polynucleotide, polypeptide, etc.) has been altered by human intervention. The term recombinant can also refer to an organism that harbors recombinant material, for example, a plant that comprises a recombinant nucleic acid is considered a recombinant plant.

As used herein, the term “sequence identity” refers to the extent to which two optimally aligned polynucleotide sequences or two optimally aligned polypeptide sequences are identical. An optimal sequence alignment is created by manually aligning two sequences, e.g., a reference sequence and another sequence, to maximize the number of nucleotide matches in the sequence alignment with appropriate internal nucleotide insertions, deletions, or gaps.

As used herein, the term “percent sequence identity” or “percent identity” or “% identity” is the identity fraction multiplied by 100. The “identity fraction” for a sequence optimally aligned with a reference sequence is the number of nucleotide matches in the optimal alignment, divided by the total number of nucleotides in the reference sequence, e.g., the total number of nucleotides in the full length of the entire reference sequence. Thus, one embodiment of the invention provides a DNA molecule comprising a sequence that, when optimally aligned to a sequence selected from SEQ ID NOs: 1-7, 86-89, 95 and 105 has at least about 85 percent identity, at least about 86 percent identity, at least about 87 percent identity, at least about 88 percent identity, at least about 89 percent identity, at least about 90 percent identity, at least about 91 percent identity, at least about 92 percent identity, at least about 93 percent identity, at least about 94 percent identity, at least about 95 percent identity, at least about 96 percent identity, at least about 97 percent identity, at least about 98 percent identity, at least about 99 percent identity, or at least about 100 percent identity to a sequence selected from SEQ ID NOs: 1-7, 86-89, 95 and 105.

A “transgene” refers to a transcribable DNA molecule heterologous to a host cell at least with respect to its location in the host cell genome and/or a transcribable DNA molecule artificially incorporated into a host cell's genome in the current or any prior generation of the cell.

“Transgenic plant” refers to a plant that comprises within its cells a heterologous polynucleotide. In some embodiments, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to refer to any cell, cell line, callus, tissue, plant part or plant, the genotype of which has been altered by the presence of heterologous nucleic acid including those transgenic organisms or cells initially so altered, as well as those created by crosses or asexual propagation from the initial transgenic organism or cell. The term “transgenic” as used herein does not encompass the alteration of the genome (chromosomal or extrachromosomal) by conventional plant breeding methods (e.g., crosses) or by naturally occurring events such as random cross-fertilization, non-recombinant viral infection, non-recombinant bacterial transformation, non-recombinant transposition, or spontaneous mutation.

As used herein, a “recombinant DNA molecule” is a DNA molecule comprising a combination of DNA molecules that would not naturally occur together without human intervention. For instance, a recombinant DNA molecule may be a DNA molecule that is comprised of at least two DNA molecules heterologous with respect to each other, a DNA molecule that comprises a DNA sequence that deviates from DNA sequences that exist in nature, a DNA molecule that comprises a synthetic DNA sequence or a DNA molecule that has been incorporated into a host cell's DNA by genetic transformation or gene editing.

Methods involving transient transformation or stable integration of any nucleic acid molecule into any plant or plant cell are provided herein. As used herein, “stable integration” or “stably integrated” on “in planta transformation” refers to a transfer of DNA into genomic DNA of a targeted cell or plant that allows the targeted cell or plant to pass the transferred DNA to the next generation of the transformed organism. Stable transformation requires the integration of transferred DNA within the reproductive cell(s) of the transformed organism. As used herein, “transiently transformed” or “transient transformation” refers to a transfer of DNA into a cell that is not transferred to the next generation of the transformed organism. In one aspect, a method stably transforms a plant cell or plant with one or more nucleic acid molecules provided herein. In another aspect, a method transiently transforms a plant cell or plant with one or more nucleic acid molecules provided herein.

Numerous methods for transforming cells with a recombinant nucleic acid molecule or construct are known in the art, which can be used according to methods of the present application. Any suitable method or technique for transformation of a cell known in the art can be used according to present methods. Effective methods for transformation of plants include bacterially mediated transformation, such as Agrobacterium-mediated or Rhizobium-mediated transformation and microprojectile bombardment-mediated transformation. A variety of methods are known in the art for transforming explants with a transformation vector via bacterially mediated transformation or microprojectile bombardment and then subsequently culturing, etc., those explants to regenerate or develop transgenic plants.

In an aspect, a method comprises providing a cell with a nucleic acid molecule via Agrobacterium-mediated transformation. In an aspect, a method comprises providing a cell with a nucleic acid molecule via polyethylene glycol-mediated transformation. In an aspect, a method comprises providing a cell with a nucleic acid molecule via biolistic transformation. In an aspect, a method comprises providing a cell with a nucleic acid molecule via liposome-mediated transfection. In an aspect, a method comprises providing a cell with a nucleic acid molecule via viral transduction. In an aspect, a method comprises providing a cell with a nucleic acid molecule via use of one or more delivery particles. In an aspect, a method comprises providing a cell with a nucleic acid molecule via microinjection. In an aspect, a method comprises providing a cell with a nucleic acid molecule via electroporation.

In an aspect, a nucleic acid molecule is provided to a cell via a method selected from the group consisting of Agrobacterium-mediated transformation, polyethylene glycol-mediated transformation, biolistic transformation, liposome-mediated transfection, viral transduction, the use of one or more delivery particles, microinjection, and electroporation.

Other methods for transformation, such as vacuum infiltration, pressure, sonication, and silicon carbide fiber agitation, are also known in the art and envisioned for use with any method provided herein.

Methods of transforming cells are well known by persons of ordinary skill in the art. For instance, specific instructions for transforming plant cells by microprojectile bombardment with particles coated with recombinant DNA (e.g., biolistic transformation) are found in U.S. Pat. Nos. 5,550,318; 5,538,880 6,160,208; 6,399,861; and 6,153,812 and Agrobacterium-mediated transformation is described in U.S. Pat. Nos. 5,159,135; 5,824,877; 5,591,616; 6,384,301; 5,750,871; 5,463,174; and 5,188,958, all of which are incorporated herein by reference. Additional methods for transforming plants can be found in, for example, Compendium of Transgenic Crop Plants (2009) Blackwell Publishing. Any appropriate method known to those skilled in the art can be used to transform a plant cell with any of the nucleic acid molecules provided herein.

Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration).

Delivery vehicles, vectors, particles, nanoparticles, formulations and components thereof for expression of one or more elements of a nucleic acid molecule are as used in WO 2014/093622. In an aspect, a method of providing a nucleic acid molecule or a protein to a cell comprises delivery via a delivery particle. In an aspect, a method of providing a nucleic acid molecule to a plant cell or plant comprises delivery via a delivery vesicle. In an aspect, a delivery vesicle is selected from the group consisting of an exosome and a liposome. In an aspect, a method of providing a nucleic acid molecule to a plant cell or plant comprises delivery via a viral vector. In an aspect, a viral vector is selected from the group consisting of an adenovirus vector, a lentivirus vector, and an adeno-associated viral vector. In another aspect, a method providing a nucleic acid molecule to a plant cell or plant comprises delivery via a nanoparticle. In an aspect, a method providing a nucleic acid molecule to a plant cell or plant comprises microinjection. In an aspect, a method providing a nucleic acid molecule to a plant cell or plant comprises polycations. In an aspect, a method providing a nucleic acid molecule to a plant cell or plant comprises a cationic oligopeptide.

In an aspect, a delivery particle is selected from the group consisting of an exosome, an adenovirus vector, a lentivirus vector, an adeno-associated viral vector, a nanoparticle, a polycation, and a cationic oligopeptide. In an aspect, a method provided herein comprises the use of one or more delivery particles. In another aspect, a method provided herein comprises the use of two or more delivery particles. In another aspect, a method provided herein comprises the use of three or more delivery particles.

Suitable agents to facilitate transfer of nucleic acids into a plant cell include agents that increase permeability of the exterior of the plant or that increase permeability of plant cells to oligonucleotides or polynucleotides. Such agents to facilitate transfer of the composition into a plant cell include a chemical agent, or a physical agent, or combinations thereof. Chemical agents for conditioning includes (a) surfactants, (b) organic solvents, aqueous solutions, or aqueous mixtures of organic solvents, (c) oxidizing agents, (e) acids, (f) bases, (g) oils, (h) enzymes, or combinations thereof.

Organic solvents useful in conditioning a plant to permeation by polynucleotides include DMSO, DMF, pyridine, N-pyrrolidine, hexamethylphosphoramide, acetonitrile, dioxane, polypropylene glycol, other solvents miscible with water or that will dissolve phosphonucleotides in non-aqueous systems (such as is used in synthetic reactions). Naturally derived or synthetic oils with or without surfactants or emulsifiers can be used, e.g., plant-sourced oils, crop oils (such as those listed in the 9th Compendium of Herbicide Adjuvants, publicly available on line at www(dot)herbicide(dot)adjuvants(dot)com) can be used, e.g., paraffinic oils, polyol fatty acid esters, or oils with short-chain molecules modified with amides or polyamines such as polyethyleneimine or N-pyrrolidine.

Examples of useful surfactants include sodium or lithium salts of fatty acids (such as tallow or tallowamines or phospholipids) and organosilicone surfactants. Other useful surfactants include organosilicone surfactants including nonionic organosilicone surfactants, e.g., trisiloxane ethoxylate surfactants or a silicone polyether copolymer such as a copolymer of polyalkylene oxide modified heptamethyl trisiloxane and allyloxypolypropylene glycol methylether (commercially available as Silwet® L-77).

Useful physical agents can include (a) abrasives such as carborundum, corundum, sand, calcite, pumice, garnet, and the like, (b) nanoparticles such as carbon nanotubes or (c) a physical force. Carbon nanotubes are disclosed by Kam et. al. (2004) Am. Chem. Soc, 126 (22):6850-6851, Liu et. al. (2009) Nano Lett, 9(3): 1007-1010, and Khodakovskaya et. al. (2009) ACS Nano, 3(10):3221-3227. Physical force agents can include heating, chilling, the application of positive pressure, or ultrasound treatment. Embodiments of the method can optionally include an incubation step, a neutralization step (e.g., to neutralize an acid, base, or oxidizing agent, or to inactivate an enzyme), a rinsing step, or combinations thereof. The methods of the invention can further include the application of other agents which will have enhanced effect due to the silencing of certain genes. For example, when a polynucleotide is designed to regulate genes that provide herbicide resistance, the subsequent application of the herbicide can have a dramatic effect on herbicide efficacy.

Agents for laboratory conditioning of a plant cell to permeation by polynucleotides include, e.g., application of a chemical agent, enzymatic treatment, heating or chilling, treatment with positive or negative pressure, or ultrasound treatment. Agents for conditioning plants in a field include chemical agents such as surfactants and salts.

In an aspect, a transformed or transfected cell is a plant cell. Recipient plant cell or explant targets for transformation include, but are not limited to, a seed cell, a fruit cell, a leaf cell, a callus cell, a cotyledon cell, a hypocotyl cell, a meristem cell, an embryo cell, an endosperm cell, a root cell, a shoot cell, a stem cell, a pod cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, a phloem cell, a bud cell, or a vascular tissue cell. In another aspect, this disclosure provides a plant chloroplast. In a further aspect, this disclosure provides an epidermal cell, a guard cell, a trichome cell, a root hair cell, a storage root cell, or a tuber cell. In another aspect, this disclosure provides a protoplast. In another aspect, this disclosure provides a plant callus cell. Any cell from which a fertile plant can be regenerated is contemplated as a useful recipient cell for practice of this disclosure. Callus can be initiated from various tissue sources, including, but not limited to, immature embryos or parts of embryos, seedling apical meristems, microspores, and the like. Those cells which are capable of proliferating as callus can serve as recipient cells for transformation. Practical transformation methods and materials for making transgenic plants of this disclosure (e.g., various media and recipient target cells, transformation of immature embryos, and subsequent regeneration of fertile transgenic plants) are disclosed, for example, in U.S. Pat. Nos. 6,194,636 and 6,232,526 and U. S. Patent Application Publication 2004/0216189, all of which are incorporated herein by reference. Transformed explants, cells or tissues can be subjected to additional culturing steps, such as callus induction, selection, regeneration, etc., as known in the art. Transformed cells, tissues or explants containing a recombinant DNA insertion can be grown, developed or regenerated into transgenic plants in culture, plugs or soil according to methods known in the art. In one aspect, this disclosure provides plant cells that are not reproductive material and do not mediate the natural reproduction of the plant. In another aspect, this disclosure also provides plant cells that are reproductive material and mediate the natural reproduction of the plant. In another aspect, this disclosure provides plant cells that cannot maintain themselves via photosynthesis. In another aspect, this disclosure provides somatic plant cells. Somatic cells, contrary to germline cells, do not mediate plant reproduction. In one aspect, this disclosure provides a non-reproductive plant cell.

In planta protein expression from transgenes is subjected to complex regulatory mechanisms and can be manipulated through different approaches. Modulation of translational efficiency by introducing contextual nucleotides flanking the translation initiator codon can be employed as one such approach for enhancing protein accumulation in planta. The Kozak sequence is a nucleic acid motif functioning as the protein translation initiation site in eukaryotic mRNA transcripts (Kozak M., 1987 and 1989). It regulates the specificity and the efficiency of the initiation of translation. It mediates the recruitment and assembly of the ribosome onto the mRNA and in the proper AUG start codon recognition to initiate translation. Variation in a native gene's Kozak sequence alters the efficiency or strength of the translation of an mRNA, directly impacting how much protein is made from a given individual mRNA strand. The Kozak consensus sequence varies slightly across species and is typically contained within 5-8 base pairs upstream and downstream of the ATG start codon. In the embodiments described herein, the A nucleotide of the start codon “ATG” is delineated as +1 with the preceding base being labeled as −1. Variations within the Kozak sequence effects mRNA translation. Kozak sequence strength herein refers to the favorability of initiation, affecting mRNA translation efficiency and how much protein is synthesized from a given mRNA. Learnings from the Kozak sequence analysis described in Example 1 and 2 is used to optimize nucleotide sequence (−9 to +6) around ATG-start codon of a transgene so as to optimize the Kozak for desired translation efficiency in planta.

In one aspect the optimized Kozak sequence increases protein accumulation in the edited eukaryotic cell as compared to the control eukaryotic cell. In one aspect the increase in protein accumulation is at least 20%. In one aspect the increase in protein accumulation is at least 30%. In one aspect the increase in protein accumulation is at least 40%. In one aspect the increase in protein accumulation is at least 50%. In one aspect the increase in protein accumulation is at least 60%. In one aspect the increase in protein accumulation is at least 70%. In one aspect the increase in protein accumulation is at least 80%. In one aspect the increase in protein accumulation is at least 90%. In one aspect the increase in protein accumulation is at least 100%. In one aspect the increase in protein accumulation is at least 200%. In one aspect the increase in protein accumulation is at least 300%. In one aspect the increase in protein accumulation is at least 400%. In one aspect the increase in protein accumulation is at least 500%. In one aspect the increase in protein accumulation is at least 1000%. In one aspect the increase in protein accumulation is at least 1500%. In one aspect the increase in protein accumulation is at least 2000%.

In one aspect the optimized Kozak sequence decreases protein accumulation in the edited eukaryotic cell as compared to the control eukaryotic cell. In one aspect the decrease in protein accumulation is at least 20%. In one aspect the decrease in protein accumulation is at least 30%. In one aspect the decrease in protein accumulation is at least 40%. In one aspect the decrease in protein accumulation is at least 50%. In one aspect the decrease in protein accumulation is at least 60%. In one aspect the decrease in protein accumulation is at least 70%. In one aspect the decrease in protein accumulation is at least 80%. In one aspect the decrease in protein accumulation is at least 90%. In one aspect the decrease in protein accumulation is at least 95%. In one aspect the decrease in protein accumulation is at least 100%.

In one aspect the optimized Kozak sequence decreases protein accumulation in the edited eukaryotic cell by 2-fold. In one aspect the optimized Kozak sequence decreases protein accumulation in the edited eukaryotic cell by 3-fold. In one aspect the optimized Kozak sequence decreases protein accumulation in the edited eukaryotic cell by 4-fold. In one aspect the optimized Kozak sequence decreases protein accumulation in the edited eukaryotic cell by 5-fold.

N-terminal amino acids (for e.g.: 2 to 8 amino acids at the N terminus of a target protein) have been known to modulate protein stability thereby affecting protein accumulation. For example, computational analysis of 236 highly abundant plant (angiosperm) proteins revealed that the three downstream codons from bases +4 to +12 (following the initiator codon ATG)—GCT TCC TCC- and the corresponding N-terminal amino acid residues (Ala2-Ser3-Ser4) are highly conserved (Sawant et al., 1999, 2001). Without being bound by any theory, it has been hypothesized that the efficient ribosomal recruitment at the ATG initiator involves an interaction between the +4 to +11 positions and the 48S pre-initiation complex in plants (Sawant et al., 2001). Of the 236 highly expressed proteins (Sawant et al., 2001), 46% had Met1-Ala2, 18% had Met1-Ala2-Ser3, 17% had Met1-Ala2-X3-Ser4, and 14% had Met1-Ala2-Ser3-Ser4 as the N-terminal amino acids. Similarly, the preference for Ala amino acid at the second position following the initial Met for majority of plant protein sequences has been also reported by other studies (Shemesh et al., 2010; Joshi et al., 1997; Lukaszewicz et al., 2000). The preference for Ser and Leu amino acid residues at the third and fourth positions following the initial Met has been also observed in eukaryotic proteins (Shemesh et al., 2010). The prevalence of the preferred amino acid in evolutionarily stable proteins might indicate a role in gene expression. Therefore, introduction of conserved nucleotide codons at specific positions for preferred amino acid residues at the N-terminus of proteins can improve protein synthesis efficiency for recombinant proteins in plants.

“Editing enzymes” refer to sequence-specific genome modification enzymes that may be used to introduce one or more insertions, deletions, substitutions, base modifications in a genomic sequence. In some embodiments, an editing enzyme can include, but is not limited to, an RNA-guided nuclease editing system, such as a CRISPR associated nuclease. CRISPR nucleases and their cognate guide nucleic acid when expressed or introduced as a system in a cell can modify a target nucleic acid in a sequence specific manner. In some embodiments, the CRISPR associated nuclease is selected from a Type I CRISPR-Cas system, a Type II CRISPR-Cas system, a Type III CRISPR-Cas system, a Type IV CRISPR-Cas system, Type V CRISPR-Cas system, or a Type VI CRISPR-Cas system. Non-limiting examples of CRISPR associated nucleases include Cas1, Cas1B, Cas2, Cas3, Cas4, Cas5, Cash, Cas7, Cas8, Cas9 (also known as Csn1 and Csx12), Cas1O, Cas 12a (also known as Cpf1), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1, Csx16, CsaX, Csx3, Csx1, Csx15, Csf1, Csf2, Csf3, Csf4, CasX, CasY, and Mad7 Other examples of editing enzymes include meganucleases, zinc finger nucleases (ZFNs), and transcription activator-like effector nucleases (TALENs). In some embodiments, an editing enzyme can comprise one or more sequence-specific nucleic acid binding domains (DNA binding domains) that can be from, for example, CRISPR nuclease effector protein (e.g., a Cas9, a Cas 12a), a zinc finger protein, and/or a transcription activator-like effector protein (TALE) and an effector domain that modifies the DNA. Examples of effector domains include cleavage domains (e.g., nucleases) including, but not limited to, an endonuclease (e.g., FokI), a deaminase (e.g., a cytosine deaminase, an adenine deaminase), a uracil glycosylase inhibitor (UGI), a reverse transcriptase, a Dna2 polypeptide, and/or a 5′ flap endonuclease (FEN). In some embodiments the editing enzyme is a CRISPR associated nickase for e.g.: Cas9 nickase, or a Cas12a nickase.

In one embodiment, the editing enzyme is a Cas 12a nuclease. In an aspect, the Cas12a provided herein is a Lachnospiraceae bacterium Cas12a (LbCas12a) nuclease. In another aspect, a Cas12a nuclease provided herein is a Francisella novicida Cas12a (FnCas12a).

In some embodiments, the editing enzyme is a base editor (BE). In some embodiments, the base editor is a cytosine based editor (CBE), which changes a C:G pair to a T:A pair in a targeting window. A CBE comprises a deaminase protein domain (e.g. APOBEC domain) fused to a nuclease (eg. Cas9, Cas9 nickase). In addition, the CBE can include uracil glycosylase inhibitor (UGI) domain to help facilitate the repair of the modification towards a non-cytosine base change (see US20210230577). In some embodiments, the base editor is a adenine based editor (ABE), which changes an A:T pair to a G:C pair in a targeting window. An ABE comprises an adenine deaminase (e.g., ecTadA) fused to a nuclease (e.g. Cas9, Cas9 nickase) (see US20210317440, Gaudelli et. al., Nature 551, 464-471 (2017).

In some embodiments, the editing enzyme is a Prime Editor (PE). Prime editing is a genome editing method that directly writes new genetic information into a specified DNA site using a nucleic acid programmable DNA binding protein (napDNAbp) (eg: Cas9) working in association with a polymerase wherein the prime editing system is programmed with a specialized prime editing (PE) guide RNA (“PEgRNA”) that both specifies the target site and templates the synthesis of the desired edit (see WO2020191248) In one embodiment, the term “prime editor” refers to fusion constructs comprising a napDNAbp (e.g., Cas9 nickase) and a reverse transcriptase and is capable of carrying out prime editing on a target nucleotide sequence in the presence of a pegRNA (or “extended guide RNA”). The term “prime editor” may refer to the fusion protein or to the fusion protein complexed with a pegRNA, and/or further complexed with a second-strand nicking sgRNA. In other embodiments, the reverse transcriptase component of the “primer editor” may be provided in trans.

CRISPR associated nucleases, require another non-coding nucleotide component, referred to as a guide nucleic acid or guide RNA, to have functional activity. When a CRISPR effector protein and a guide RNA form a complex, the whole system is called a “ribonucleoprotein.” Ribonucleoproteins provided herein can also comprise additional nucleic acids or proteins.

Guide nucleic acid molecules provided herein can be DNA, RNA, or a combination of DNA and RNA. As used herein, a “guide RNA” or “gRNA” refers to an RNA that recognizes a target DNA sequence and directs, or “guides”, a CRISPR nuclease to the target DNA sequence. A guide RNA for Cas9 is comprised of a region that is complementary to the target DNA (referred to as the crRNA) and a region that binds the CRISPR effector protein (referred to as the tracrRNA). Cas12a does not require a tracrRNA, therefore, in an aspect when utilizing Cas12a, the gRNA comprises a crRNA. The Cas12a crRNA comprises a repeat sequence and a spacer sequence which is complementary to the target sequence. A “single-chain guide RNA” (or “sgRNA”) is a RNA molecule comprising a crRNA covalently linked a tracrRNA by a linker sequence, which may be expressed as a single RNA transcript or molecule. A guide RNA may be a single RNA molecule (sgRNA) or two separate RNAs molecules (a 2-piece gRNA). In some embodiments a gRNA may be a split gRNA. In some embodiments a gRNA may be an engineered prime editing guide RNA (pegRNA) that is used in conjunction with a Prime editor and comprises an RNA template (pegRNA) for a reverse transcriptase. In some embodiments, the gRNA is a split pegRNA comprising a prime editing tracrRNA (petracrRNA) and a crRNA.

A prerequisite for cleavage of the target site by a CRIPSR associated nuclease in the presence of a conserved protospacer-adjacent motif (PAM) adjacent to the target sequence. For Cas9 the PAM site is downstream of the target site which usually has the sequence 5-NGG-3 but less frequently NAG. Specificity is provided by the “seed sequence” approximately 12 bases upstream of the PAM, which must match between the RNA and target DNA. The PAM motif of Cas12a is upstream of the target site and for Cas12a orthologs LbCas12a and AsCas12a (Acidaminococcus sp. BV3L6 Cas12a), the PAM sequence is 5-TTTV-3 where V can be A, C, or G. LbCas12a-RR is a variant of LbCas12a that comprises the mutations G532R/K595R and recognizes the PAM sequence 5-TYCV-3 where Y can be C or T (Gao et al., 2017). The PAM motif for FnCas12a is 5-TTV-3. As used herein, a “protospacer adjacent motif” (PAM) refers to a 2-6 base pair DNA sequence immediately upstream or downstream of a target sequence of a CRISPR complex.

While not being limited by any particular scientific theory, a CRISPR nuclease forms a complex with a guide RNA (gRNA), which hybridizes with a complementary target site, thereby guiding the CRISPR nuclease to the target site. In class II CRISPR-Cas systems, CRISPR arrays, including spacers, are transcribed during encounters with recognized invasive DNA and are processed into small interfering CRISPR RNAs (crRNAs). The crRNA comprises a repeat sequence and a spacer sequence which is complementary to a specific protospacer sequence in an invading pathogen. The spacer sequence can be designed to be complementary to target sequences of a target site in a eukaryotic genome.

As used herein, a “target sequence” refers to a selected sequence or region of a DNA molecule in which a modification (e.g., cleavage, insertion, deletion, substitution site-directed integration) is desired. A target sequence comprises a target site.

As used herein, a “target site” refers to the portion of a target sequence that is modified (e.g., cleaved) by a CRISPR nuclease. In contrast to a non-target nucleic acid (e.g., non-target ssDNA) or non-target region, a target site comprises significant complementarity to a guide nucleic acid or a guide RNA.

In an aspect, a target site is 100% complementary to a guide nucleic acid. In another aspect, a target site is 99% complementary to a guide nucleic acid. In another aspect, a target site is 98% complementary to a guide nucleic acid. In another aspect, a target site is 97% complementary to a guide nucleic acid. In another aspect, a target site is 96% complementary to a guide nucleic acid. In another aspect, a target site is 95% complementary to a guide nucleic acid. In another aspect, a target site is 94% complementary to a guide nucleic acid. In another aspect, a target site is 93% complementary to a guide nucleic acid. In another aspect, a target site is 92% complementary to a guide nucleic acid. In another aspect, a target site is 91% complementary to a guide nucleic acid. In another aspect, a target site is 90% complementary to a guide nucleic acid. In another aspect, a target site is 85% complementary to a guide nucleic acid. In another aspect, a target site is 80% complementary to a guide nucleic acid.

In an aspect, a target site comprises at least one PAM site. In an aspect, a target site is adjacent to a nucleic acid sequence that comprises at least one PAM site. In another aspect, a target site is within 5 nucleotides of at least one PAM site. In a further aspect, a target site is within 10 nucleotides of at least one PAM site. In another aspect, a target site is within 15 nucleotides of at least one PAM site. In another aspect, a target site is within 20 nucleotides of at least one PAM site. In another aspect, a target site is within 25 nucleotides of at least one PAM site. In another aspect, a target site is within 30 nucleotides of at least one PAM site.

In an aspect, a target site is positioned within genic DNA. In another aspect, a target site is positioned within a gene. In another aspect, a target site is positioned within a gene of interest. In another aspect, a target site is positioned within the promoter of a gene. In another aspect, a target site is positioned adjacent to a Kozak sequence. In another aspect, a target site comprises a Kozak sequence. In another aspect, a target site is positioned within an exon of a gene. In another aspect, a target site is positioned within an intron of a gene. In another aspect, a target site is positioned within 5′-UTR of a gene. In another aspect, a target site is positioned within intergenic DNA.

In an aspect, a target sequence comprises genomic DNA. In an aspect, a target sequence is positioned within a nuclear genome. In an aspect, a target sequence comprises chromosomal DNA. In an aspect, a target sequence comprises plasmid DNA. In an aspect, a target sequence is positioned within a plasmid. In an aspect, a target sequence comprises mitochondrial DNA. In an aspect, a target sequence is positioned within a mitochondrial genome. In an aspect, a target sequence comprises plastid DNA. In an aspect, a target sequence is positioned within a plastid genome. In an aspect, a target sequence comprises chloroplast DNA. In an aspect, a target sequence is positioned within a chloroplast genome. In an aspect, a target sequence is positioned within a genome selected from the group consisting of a nuclear genome, a mitochondrial genome, and a plastid genome.

As used herein, a “template nucleic acid molecule”, a “repair template”, a “donor template” refers to a nucleic acid molecule that comprises a nucleic acid sequence that is to be inserted into a target DNA molecule. In an aspect, a template nucleic acid molecule comprises single-stranded DNA. In another aspect, a template nucleic acid molecule comprises double-stranded DNA. In a further aspect, a template nucleic acid molecule comprises single-stranded RNA. In yet another aspect, a template nucleic acid molecule comprises double-stranded RNA. In another aspect, a template nucleic acid molecule comprises DNA and RNA. In an aspect the template nucleic acid molecule comprises at least one nucleotide modification when compared to the nucleotide sequence to be edited. In a preferred embodiment, the template nucleic acid sequences comprises a Kozak sequence. In an aspect, a template nucleic acid molecule comprises one or two homology arms flanking the desired sequence to promote the targeted insertion event through homologous recombination (HR) and/or homology-directed repair (HDR).

Endogenous DNA repair acting upon a targeted DSB drives the template integration process. Depending on the repair pathway, integration can occur through homology directed repair (HDR) or non-homologous end joining (NHEJ) (Schmidt et al., 2019; Van Eck, 2020). In HDR, the heterologous DNA segment is flanked by homologous regions between the chromosome and integrating DNA. Homologous recombination between the donor and the chromosome provides scarless chromosomal integration. On the other hand, NHEJ uses no or very short homologies for repair. NHEJ heals DSBs more efficiently but is often accompanied by point mutations at the junctions. In some instances, integrations that were initiated by HDR, are completed by NHEJ on the other arm. These scenarios can be created by the somatic HDR pathway synthesis-dependent strand-annealing (SDSA) or possibly by a combination of various other DNA repair mechanisms (Schmidt et al., 2019).

The methods described herein may be utilized to regulate the accumulation of proteins encoded by genes of agronomic interest. In some embodiments, the native Kozak sequences of genes of agronomic interest may be edited to confer features of strong mRNA translational efficacy Kozak consensus sequences. In some embodiments, the native Kozak sequences of genes of agronomic interest may be edited to confer features of adequate mRNA translational efficacy Kozak consensus sequences. In some embodiments, the native Kozak sequences of genes of agronomic interest may be edited to confer features of weak mRNA translational efficacy Kozak consensus sequences. In some embodiments, the native Kozak sequences of genes of agronomic interest may be edited to remove features of strong mRNA translational efficacy Kozak consensus sequences. In some embodiments, the native Kozak sequences of genes of agronomic interest may be edited to remove features of weak mRNA translational efficacy Kozak consensus sequences.

As used herein, the term “native” refers to a sequence that is the endogenous sequence, a sequence that is identical to the endogenous sequence, or a sequence that has not been edited.

As used herein, the term “gene of agronomic interest” refers to a transcribable DNA molecule that, when expressed in a particular plant tissue, cell, or cell type, confers a desirable characteristic. The product of a gene of agronomic interest may act within the plant in order to cause an effect upon the plant morphology, physiology, growth, development, yield, grain composition, nutritional profile, disease or pest resistance, and/or environmental or chemical tolerance or may act as a pesticidal agent in the diet of a pest that feeds on the plant. A beneficial agronomic trait may include, for example, but is not limited to, herbicide tolerance, insect control, modified yield, disease resistance, pathogen resistance, modified plant growth and development, modified starch content, modified oil content, modified fatty acid content, modified protein content, modified fruit ripening, enhanced animal and human nutrition, biopolymer productions, environmental stress resistance, pharmaceutical peptides, improved processing qualities, improved flavor, hybrid seed production utility, improved fiber production, augmented carbon sequestration, and desirable biofuel production.

Examples of genes of agronomic interest known in the art include those for herbicide resistance (U.S. Pat. Nos. 6,803,501; 6,448,476; 6,248,876; 6,225,114; 6,107,549; 5,866,775; 5,804,425; 5,633,435; and 5,463,175), increased yield (U.S. Pat. Nos. RE38,446; 6,716,474; 6,663,906; 6,476,295; 6,441,277; 6,423,828; 6,399,330; 6,372,211; 6,235,971; 6,222,098; and 5,716,837), insect control (U.S. Pat. Nos. 6,809,078; 6,713,063; 6,686,452; 6,657,046; 6,645,497; 6,642,030; 6,639,054; 6,620,988; 6,593,293; 6,555,655; 6,538,109; 6,537,756; 6,521,442; 6,501,009; 6,468,523; 6,326,351; 6,313,378; 6,284,949; 6,281,016; 6,248,536; 6,242,241; 6,221,649; 6,177,615; 6,156,573; 6,153,814; 6,110,464; 6,093,695; 6,063,756; 6,063,597; 6,023,013; 5,959,091; 5,942,664; 5,942,658, 5,880,275; 5,763,245; and 5,763,241), fungal disease resistance (U.S. Pat. Nos. 6,653,280; 6,573,361; 6,506,962; 6,316,407; 6,215,048; 5,516,671; 5,773,696; 6,121,436; 6,316,407; and 6,506,962), virus resistance (U.S. Pat. Nos. 6,617,496; 6,608,241; 6,015,940; 6,013,864; 5,850,023; and 5,304,730), nematode resistance (U.S. Pat. No. 6,228,992), bacterial disease resistance (U.S. Pat. No. 5,516,671), plant growth and development (U.S. Pat. Nos. 6,723,897 and 6,518,488), starch production (U.S. Pat. Nos. 6,538,181; 6,538,179; 6,538,178; 5,750,876; 6,476,295), modified oils production (U.S. Pat. Nos. 6,444,876; 6,426,447; and 6,380,462), high oil production (U.S. Pat. Nos. 6,495,739; 5,608,149; 6,483,008; and 6,476,295), modified fatty acid content (U.S. Pat. Nos. 6,828,475; 6,822,141; 6,770,465; 6,706,950; 6,660,849; 6,596,538; 6,589,767; 6,537,750; 6,489,461; and 6,459,018), high protein production (U.S. Pat. No. 6,380,466), fruit ripening (U.S. Pat. No. 5,512,466), enhanced animal and human nutrition (U.S. Pat. Nos. 6,723,837; 6,653,530; 6,5412,59; 5,985,605; and 6,171,640), biopolymers (U.S. Pat. Nos. RE37,543; 6,228,623; and 5,958,745, and 6,946,588), environmental stress resistance (U.S. Pat. No. 6,072,103), pharmaceutical peptides and secretable peptides (U.S. Pat. Nos. 6,812,379; 6,774,283; 6,140,075; and 6,080,560), improved processing traits (U.S. Pat. No. 6,476,295), improved digestibility (U.S. Pat. No. 6,531,648) low raffinose (U.S. Pat. No. 6,166,292), industrial enzyme production (U.S. Pat. No. 5,543,576), improved flavor (U.S. Pat. No. 6,011,199), nitrogen fixation (U.S. Pat. No. 5,229,114), hybrid seed production (U.S. Pat. No. 5,689,041), fiber production (U.S. Pat. Nos. 6,576,818; 6,271,443; 5,981,834; and 5,869,720) and biofuel production (U.S. Pat. No. 5,998,700).

### SPECIFIC EMBODIMENTS

The following embodiments are provided by way of illustration, and are not intended to be limiting of the invention, unless specified.

A first embodiment relates to a method of altering protein accumulation in an edited eukaryotic cell, the method comprising editing the Kozak sequence of a nucleic acid molecule encoding the protein at one or more nucleotides of positions −9, −8, −7, −6, −5, −4, −3, −2, −1, +4, and +5 of the Kozak sequence, where the “A” nucleotide of the ATG start codon is delineated as +1, to generate an edited nucleic acid molecule comprising an edited Kozak sequence, wherein the edited eukaryotic cell comprising the edited nucleic acid molecule exhibits a statistically significant alteration of the accumulation of the protein as compared to the accumulation of the protein within a control eukaryotic cell comprising a reference nucleic acid sequence.

A second embodiment relates to the method of embodiment 1, wherein the protein accumulation is increased in the edited eukaryotic cell as compared to the control eukaryotic cell.

A third embodiment relates to the method of embodiment 2, wherein the protein accumulation is increased by at least 20%.

A fourth embodiment relates to the method of embodiment 1, wherein the protein accumulation is decreased in the edited eukaryotic cell as compared to the control eukaryotic cell.

A fifth embodiment relates to the method of embodiment 4, wherein the protein accumulation is decreased by at least 20%.

A sixth embodiment relates to the method of embodiment 4, wherein the protein accumulation is decreased by at least 2-fold.

A seventh embodiment relates to the method of embodiment 1, wherein the nucleic acid molecule is an endogenous nucleic acid molecule.

An eight embodiment relates to the method of embodiment 1, wherein the nucleic acid molecule is a transgenic nucleic acid molecule.

A ninth embodiment relates to the method of embodiment 1, wherein accumulation of mRNA transcribed from the edited nucleic acid molecule in the edited eukaryotic cell is increased as compared to accumulation of mRNA transcribed from the reference sequence in the control eukaryotic cell.

A tenth embodiment relates to the method of embodiment 1, wherein accumulation of mRNA transcribed from the edited nucleic acid molecule in the edited eukaryotic cell is decreased as compared to accumulation of mRNA transcribed from the reference sequence in the control eukaryotic cell.

An eleventh embodiment relates to the method of embodiment 1, wherein accumulation of mRNA transcribed from the edited nucleic acid molecule in the edited eukaryotic cell is not statistically significantly different as compared to accumulation of mRNA transcribed from the reference sequence in the control eukaryotic cell.

A twelfth embodiment relates to the method of embodiment 1, wherein the eukaryotic cell is selected from the group consisting of a plant cell, a fungal cell, and an animal cell.

A thirteenth embodiment relates to the method of embodiment 12, wherein the plant cell is selected from the group consisting of a dicot cell and a monocot cell.

A fourteenth embodiment relates to the method of embodiment 12, wherein the plant cell is selected from the group consisting of a corn cell, a soybean cell, a tomato cell, a rice cell, a canola cell, a pepper cell, a wheat cell, a cucumber cell, an onion cell, an oilseed rape cell, and a cotton cell.

A fifteenth embodiment relates to method of embodiment 1, wherein the edited Kozak sequence comprises a sequence selected from the group consisting of SEQ ID NOs: 1-7, 86-89, 95 and 105.

A sixteenth embodiment relates to the method of embodiment 1, wherein the editing comprises the use of a method selected from the group consisting of template editing, base editing, and prime editing.

A seventeenth embodiment relates to the method of embodiment 1, wherein the edited Kozak sequence is a depleted Kozak sequence.

An eighteenth embodiment relates to the method of embodiment 1, wherein the protein comprises one or more N-terminal amino acid modifications.

A nineteenth embodiment relates to the method of embodiment 18, wherein the one or more N-terminal amino acid modifications introduces an N-terminal sequence selected from the group consisting of: Alanine wherein Alanine is coded by the codon GCG, Alanine wherein Alanine is coded by the codon GCT, Arginine, Methionine-Alanine-Serine-Serine wherein Alanine is coded by the codon GCG; Methionine-Alanine-Serine-Serine wherein Alanine is coded by the codon GCT; Methionine-Alanine-Alanine; Methionine-Alanine-Serine-Leucine; and Methionine-Alanine-Alanine-Leucine.

A twentieth embodiment relates to the method of embodiment 1, wherein an A or G at the −3 position is edited to a C or T.

A twenty first embodiment relates to the method of embodiments 1 or 20, wherein a G at the +4 position is edited to an A, C, or T.

A twenty second embodiment relates to the method of embodiments 1, 20 or 21, wherein a C at the −1 position is edited to an A, G, or T.

A twenty third embodiment relates to the method of embodiments 1, 20, 21, or 22, wherein a C at the −2 position is edited to an A, G, or T.

A twenty fourth embodiment relates to the method of embodiment 1, wherein an A at the −4 position is edited to a G, C, or T.

A twenty fifth embodiment relates to the method of embodiments 1 or 24, wherein an A at the −3 position is edited to a G, C, or T.

A twenty sixth embodiment relates to the method of embodiments 1, 24 or 25, wherein an A at the −2 position is edited to a G, C, or T.

A twenty seventh embodiment relates to the method of embodiments 1, 24, 25 or 26, wherein an A at the −1 position is edited to a G, C, or T.

A twenty eighth embodiment relates to the method of embodiments 1, 24, 25, 26 or 27, wherein a G at the +4 position is edited to an A, C, or T.

A twenty-ninth embodiment relates to the method of embodiments 1, 24, 25, 26, 27 or 28, wherein a C at the +5 position is edited to an A, G, or T.

A thirtieth embodiment relates to the method of embodiment 1 wherein the eukaryotic cell is a monocot cell and wherein the nucleotide at the −8 position is edited to a T.

A thirty-first embodiment relates to the method of embodiments 1 or 30 wherein the eukaryotic cell is a monocot cell and wherein the nucleotide at the −5 position is edited to an A or T.

A thirty-second embodiment relates to the method of embodiments 1, 30 or 31 wherein the eukaryotic cell is a monocot cell and wherein the nucleotide at the −4 position is edited to a T.

A thirty-third embodiment relates to the method of embodiments 1, 30, 31 or 32 wherein the eukaryotic cell is a monocot cell and wherein the nucleotide at the −3 position is edited to a T or C.

A thirty-fourth embodiment relates to the method of embodiments 1, 30, 31, 32 or 33 wherein the eukaryotic cell is a monocot cell and wherein the nucleotide at the −2 position is edited to a T or G.

A thirty-fifth embodiment relates to the method of embodiments 1, 30, 31, 32, 33 or 34 wherein the eukaryotic cell is a monocot cell and wherein the nucleotide at the +4 position is edited to an A, T or C.

A thirty-sixth embodiment relates to the method of embodiments 1, 30, 31, 32, 33, 34 or 35 wherein the eukaryotic cell is a monocot cell and wherein the nucleotide at the +5 position is edited to an G or T.

A thirty-seventh embodiment relates to the method of embodiments 1, 30, 31, 32, 33, 34, 35 or 36 wherein the eukaryotic cell is a monocot cell and wherein the nucleotide at the +6 position is edited to an A or T.

A thirty-eighth embodiment relates to the method of embodiment 1, wherein the eukaryotic cell is a dicot cell and wherein the nucleotide at the −6 position is edited to a C, G or T.

A thirty-ninth embodiment relates to the method of embodiments 1 or 38, wherein the eukaryotic cell is a dicot cell and wherein the nucleotide at the −4 position is edited to a C, G or T.

A fortieth embodiment relates to the method of embodiments 1, 38 or 39, wherein the eukaryotic cell is a dicot cell and wherein the nucleotide at the −3 position is edited to a C or T.

A forty-first embodiment relates to the method of embodiments 1, 38, 39 or 40, wherein the eukaryotic cell is a dicot cell and wherein the nucleotide at the −2 position is edited to a G or T.

A forty-second embodiment relates to the method of embodiments 1, 38, 39, 40 or 41, wherein the eukaryotic cell is a dicot cell and wherein the nucleotide at the −1 position is edited to a C, G or T.

A forty-third embodiment relates to the method of embodiments 1, 38, 39, 40, 41 or 42, wherein the eukaryotic cell is a dicot cell and wherein the nucleotide at the +4 position is edited to a C, A or T.

A forty-fourth embodiment relates to the method of embodiments 1, 38, 39, 40, 41, 42 or 43, wherein the eukaryotic cell is a dicot cell and wherein the nucleotide at the +5 position is edited to a G, A or T.

A forty-fifth embodiment relates to the method of embodiments 1, 38, 39, 40, 41, 42, 43 or 44, wherein the eukaryotic cell is a dicot cell and wherein the nucleotide at the +6 position is edited to a C or A.

A forty-sixth embodiment relates to a method of generating an edited plant, the method comprising:

providing an editing enzyme, or a nucleic acid molecule encoding the editing enzyme, to a plant cell;
generating an edit in a Kozak sequence of a nucleic acid molecule encoding a protein in the plant cell to generate an edited Kozak sequence, wherein the edit comprises editing the Kozak sequence in one or more nucleotide positions of the Kozak sequence selected from the group consisting of positions −9, −8, −7, −6, −5, −4, −3, −2, −1, +4, and +5; and regenerating an edited plant from the plant cell, wherein the edited plant comprises the edited Kozak sequence, and wherein accumulation of the protein is altered in the edited plant as compared to a control plant when grown under comparable conditions.

A forty-seventh embodiment relates to the method of embodiment 46, wherein the editing enzyme is selected from the group consisting of a Cas9 nuclease, a Cas12a nuclease, a cytosine base editor, an adenine base editor, a Cas9 nickase, and a Cas12a nickase.

A forty-eighth embodiment relates to the method of embodiment 47, wherein the editing enzyme further comprises an engineered reverse transcriptase.

A forty-ninth embodiment relates to the method of embodiment 46, wherein the method further comprises the use of a guide RNA (gRNA), or a nucleic acid molecule encoding the gRNA.

A fiftieth embodiment relates to the method of embodiment 49, wherein the gRNA is a single-gRNA (sgRNA).

A fifty-first embodiment relates to the method of embodiment 49, wherein the gRNA is a split gRNA.

A fifty-second embodiment relates to the method of embodiment 49, wherein the editing enzyme and the gRNA are provided as a ribonucleoprotein complex.

A fifty-third embodiment relates to the method of embodiment 46, wherein the providing comprises a method selected from the group consisting of polyethylene-glycol mediated protoplast transformation, Agrobacterium-mediated transformation, particle bombardment, and carbon nanoparticle delivery.

A fifty-fourth embodiment relates to the method of embodiment 46, wherein accumulation of the protein is increased in the edited plant as compared to the control plant.

A fifty-fifth embodiment relates to the method of embodiment 54, wherein accumulation of the protein is increased at least 20%.

A fifty-sixth embodiment relates to the method of embodiment 46, wherein accumulation of the protein is decreased in the edited plant as compared to the control plant.

A fifty-seventh embodiment relates to the method of embodiment 56, wherein accumulation of the protein is decreased at least 20%.

A fifty-eighth embodiment relates to the method of embodiment 46, wherein the plant cell is selected from the group consisting of a corn cell, a soybean cell, a tomato cell, a rice cell, a canola cell, a pepper cell, a wheat cell, a cucumber cell, an onion cell, an oilseed rape cell, and a cotton cell.

A fifty-ninth embodiment relates to the method of embodiment 46, wherein the plant cell is a protoplast cell or a callus cell.

A sixtieth embodiment relates to the method of embodiment 46, wherein the nucleic acid molecule is an endogenous nucleic acid molecule.

A sixty-first embodiment relates to the method of embodiment 46, wherein the nucleic acid molecule is a transgenic nucleic acid molecule.

A sixty-second embodiment relates to the method of embodiment 46, wherein the edited Kozak sequence comprises a sequence selected from the group consisting of SEQ ID NOs: 1-7, 86-89, 95 and 105.

A sixty-third embodiment relates to the method of embodiment 46, wherein the method further comprises generating an edit resulting in one or more N-terminal amino acid modifications of the protein.

A sixty-fourth embodiment relates to the method of embodiment 63, wherein the one or more N-terminal amino acid modifications introduces an N-terminal sequence selected from the group consisting of: Methionine-Alanine-Serine-Serine wherein Alanine is coded by the codon GCG; Methionine-Alanine-Serine-Serine wherein Alanine is coded by the codon GCT; Methionine-Alanine-Alanine; Methionine-Alanine-Serine-Leucine; and Methionine-Alanine-Alanine-Leucine.

A sixty-fifth embodiment relates to the method of embodiment 46, wherein an A or G at the −3 position is edited to a C or T.

A sixty-sixth embodiment relates to the method of embodiments 46 or 65, wherein a G at the +4 position is edited to an A, C, or T.

A sixty-seventh embodiment relates to the method of embodiments 46, 65 or 66, wherein a C at the −1 position is edited to an A, G, or T.

A sixty-eighth embodiment relates to the method of embodiments 46, 65, 66, or 67, wherein a C at the −2 position is edited to an A, G, or T.

A sixty-ninth embodiment relates to the method of embodiments 46, wherein an A at the −4 position is edited to a G, C, or T.

A seventieth embodiment relates to the method of embodiments 46 or 69, wherein an A at the −3 position is edited to a G, C, or T.

A seventy-first embodiment relates to the method of embodiments 46, 69 or 70, wherein an A at the −2 position is edited to a G, C, or T.

A seventy-second embodiment relates to the method of embodiments 46, 69, 70 or 71, wherein an A at the −1 position is edited to a G, C, or T.

A seventy-third embodiment relates to the method of embodiments 46, 69, 70, 71 or 72, wherein a G at the +4 position is edited to an A, C, or T.

A seventy-fourth embodiment relates to the method of embodiments 46, 69, 70, 71, 72 or 73, wherein a C at the +5 position is edited to an A, G, or T.

A seventy-fifth embodiment relates to the method of embodiment 46 wherein the plant is a monocot and wherein the nucleotide at the −8 position is edited to a T.

A seventy-sixth embodiment relates to the method of embodiments 46 or 75 wherein the plant is a monocot and wherein the nucleotide at the −5 position is edited to an A or T.

A seventy-seventh embodiment relates to the method of embodiments 46, 75 or 76 wherein the plant is a monocot and wherein the nucleotide at the −4 position is edited to a T.

A seventy-eighth embodiment relates to the method of embodiments 46, 75, 76 or 77 wherein the plant is a monocot and wherein the nucleotide at the −3 position is edited to a T or C.

A seventy-ninth embodiment relates to the method of embodiments 46, 75, 76, 77 or 78 wherein the plant is a monocot and wherein the nucleotide at the −2 position is edited to a T or G.

An eightieth embodiment relates to the method of embodiments 46, 75, 76, 77, 78 or 79 wherein the plant is a monocot and wherein the nucleotide at the +4 position is edited to an A, T or C.

An eighty-first embodiment relates to the method of embodiments 46, 75, 76, 77, 78, 79 or 80 wherein the plant is a monocot and wherein the nucleotide at the +5 position is edited to an G or T.

An eighty-second embodiment relates to the method of embodiments 46, 75, 76, 77, 78, 79, 80 or 81 wherein the plant is a monocot and wherein the nucleotide at the +6 position is edited to an A or T.

An eighty-third embodiment relates to the method of embodiment 46, wherein the plant is a dicot and wherein the nucleotide at the −6 position is edited to a C, G or T.

An eighty-fourth embodiment relates to the method of embodiments 46 or 83, wherein the plant is a dicot and wherein the nucleotide at the −4 position is edited to a C, G or T.

An eighty-fifth embodiment relates to the method of embodiments 46, 83 or 84, wherein the plant is a dicot and wherein the nucleotide at the −3 position is edited to a C or T.

An eighty-sixth embodiment relates to the method of embodiments 46, 83, 84 or 85, wherein the plant is a dicot and wherein the nucleotide at the −2 position is edited to a G or T.

An eighty-seventh embodiment relates to the method of embodiments 46, 83, 84, 85 or 86, wherein the plant is a dicot and wherein the nucleotide at the −1 position is edited to a C, G or T.

An eighty-eighth embodiment relates to the method of embodiments 46, 83, 84, 85, 86 or 87, wherein the plant is a dicot and wherein the nucleotide at the +4 position is edited to a C, A or T.

An eighty-ninth embodiment relates to the method of embodiments 46, 83, 84, 85, 86, 87 or 88, wherein the plant is a dicot and wherein the nucleotide at the +5 position is edited to a G, A or T.

A ninetieth embodiment relates to the method of embodiments 46, 83, 84, 85, 86, 87, 88 or 89, wherein the plant is a dicot and wherein the nucleotide at the +6 position is edited to a C or A.

A ninety-first embodiment relates to a prime editing guide RNA (pegRNA) sequence, wherein the pegRNA sequence is capable of directing a prime editor (PE) to a Kozak sequence of a nucleic acid molecule, and wherein the pegRNA comprises a template sequence to edit the Kozak sequence at one or more positions selected from the group consisting of positions −9, −8, −7, −6, −5, −4, −3, −2, −1, +4, and +5 as compared to a reference Kozak sequence.

A ninety-second embodiment relates to the pegRNA of embodiment 91, wherein the pegRNA is a split pegRNA.

A ninety-third embodiment relates to the pegRNA of embodiment 92, wherein the split pegRNA comprises a prime editing tracrRNA (petracrRNA) and a crRNA.

A ninety-fourth embodiment relates to the pegRNA of embodiment 91, wherein the template sequence comprises a strong Kozak sequence.

A ninety-fifth embodiment relates to the pegRNA of embodiment 94, wherein the strong Kozak sequence is selected from the group consisting of SEQ ID NOs: 1, 3, 5, 7, 86, 95 and 105.

A ninety-sixth embodiment relates to the pegRNA of embodiment 91, wherein the template sequence comprises an adequate Kozak sequence.

A ninety-seventh embodiment relates to the pegRNA of embodiment 91, wherein the template sequence comprises a weak Kozak sequence.

A ninety-eighth embodiment relates to the pegRNA of embodiment 91, wherein the template sequence comprises a depleted Kozak sequence.

A ninety-ninth embodiment relates to the pegRNA of embodiment 98, wherein the depleted Kozak sequence is selected from the group consisting of SEQ ID NOs: 2, 4, and 6.

A one hundredth embodiment relates to the pegRNA of embodiment 91, wherein the pegRNA is part of a ribonucleoprotein complex.

A one hundred first embodiment relates to the pegRNA of embodiment 100, wherein the ribonucleoprotein complex comprises either (a) a Cas9 nickase or (b) a Cas12a nickase; and (c) an engineered reverse transcriptase.

A one hundred second embodiment relates to a nucleic acid molecule encoding the pegRNA of embodiment 91.

A one hundred third embodiment relates to an edited eukaryotic cell comprising a recombinant Kozak sequence within a nucleic acid molecule encoding a target protein, wherein the recombinant Kozak sequence comprises one or more mutations as compared to a reference sequence in nucleotides at one or more positions independently selected from the group consisting of positions −9, −8, −7, −6, −5, −4, −3, −2, −1, +4, and +5, wherein the edited eukaryotic cell exhibits altered accumulation of the target protein compared to a control eukaryotic cell.

A one hundred fourth embodiment relates to the edited eukaryotic cell of embodiment 103, wherein the edited eukaryotic cell is an edited plant cell.

A one hundred fifth embodiment relates to the edited plant cell of embodiment 104, wherein the plant cell is selected from the group consisting of a corn cell, a soybean cell, a tomato cell, a rice cell, a canola cell, a pepper cell, a wheat cell, a cucumber cell, an onion cell, an oilseed rape cell, and a cotton cell.

A one hundred sixth embodiment relates to a plant, or plant part, comprising the edited plant cell of embodiment 104.

A one hundred seventh embodiment relates to a plant product comprising the edited plant cell of embodiment 104.

A one hundred eighth embodiment relates to the edited eukaryotic cell of embodiment 103, wherein the recombinant Kozak sequence comprises one or more of an A or G at the −3 position; a G at the +4 position; a C at the −1 position; and a C at the −2 position.

A one hundred ninth embodiment relates to the edited eukaryotic cell of embodiment 103, wherein the recombinant Kozak sequence comprises an C or T at the −3 position and an A, C, or T at the +4 position.

A one hundred tenth embodiment relates to edited eukaryotic cell of embodiment 103, wherein the recombinant Kozak sequence comprises one or more of a C or T at the −3 position; an A, C or T at the +4 position; an A, G or T at the −1 position; and an A, G or T at the −2 position.

A one hundred eleventh embodiment relates to the edited eukaryotic cell of embodiment 103, wherein the recombinant Kozak sequence comprises one or more of an A at the −4 position; an A at the −3 position; an A at the −2 position; an A at the −1 position; a G at the +4 position; and a C at the +5 position.

A one hundred twelfth embodiment relates to edited eukaryotic cell of embodiment 103, wherein the recombinant Kozak sequence comprises one or more of a C, T, or G at the −4 position; a C, T, or G at the −3 position; a C, T, or G at the −2 position; a C, T, or G at the −1 position; an A, C or T at the +4 position; and an A, G or T at the +5 position.

A one hundred thirteenth embodiment relates to the edited eukaryotic cell of embodiment 103, wherein the recombinant Kozak sequence comprises: (a) at least two A's between positions −4 to −1; or (b) one A between positions −4 and −1 and a G at position +4.

A one hundred fourteenth embodiment relates to the edited eukaryotic cell of embodiment 103, wherein the recombinant Kozak sequence comprises: less than two A's between positions −4 and −1 and no G at position +4.

A one hundred fifteenth embodiment relates to the edited eukaryotic cell of embodiment 103, wherein the recombinant Kozak sequence comprises a sequence selected from the group consisting of SEQ ID NOs: 2, 4, and 6.

A one hundred sixteenth embodiment relates to the edited eukaryotic cell of embodiment 103, wherein the recombinant Kozak sequence comprises a sequence selected from the group consisting of SEQ ID NOs: 1, 3, 5, 7, and 86, 95 and 105.

A one hundred seventeenth embodiment relates to the edited eukaryotic cell of embodiment 103, wherein the recombinant Kozak sequence comprises one or more of a T at the −8 position, an A or T at the −5 position, a T at the −4 position, a T or C at the −3 position, a T or G at the −2 position, an A, T or C at the +4 position, a G or T at the +5 position, and an A or T at the +6 position.

A one hundred eighteenth embodiment relates the edited eukaryotic cell of embodiment 103, wherein the recombinant Kozak sequence comprises one or more of a C, G or T at the −6 position, a C, G or T at the −4 position, a C or T at the −3 position, a G or T at the −2 position, a C, G or T at the −1 position, a C, A or T at the +4 position, a G, A or T at the +5 position, and a C or A at the +6 position.

A one hundred nineteenth embodiment relates to the edited eukaryotic cell of embodiments 103-118, wherein the nucleic acid molecule encoding the target protein encodes one or more N-terminal amino acid modifications of the target protein.

A one hundred twentieth embodiment relates to the edited eukaryotic cell of embodiment 119, wherein the one or more N-terminal amino acid modifications introduces an N-terminal sequence selected from the group consisting of: Methionine-Alanine-Serine-Serine wherein Alanine is coded by the codon GCG; Methionine-Alanine-Serine-Serine wherein Alanine is coded by the codon GCT; Methionine-Alanine-Alanine; Methionine-Alanine-Serine-Leucine; and Methionine-Alanine-Alanine-Leucine.

A one hundred twenty first embodiment relates to a recombinant DNA molecule comprising a plant expressible promoter operably linked to a heterologous nucleic acid sequence encoding a protein, wherein the nucleic acid sequence comprises a sequence selected from the group consisting of: a) a sequence with at least 90 percent sequence identity to any of SEQ ID NOs: 1-7, 86-89, 95 and 105; and b) a sequence comprising any of SEQ ID NOs: 1-7, 86-89, 95 and 105.

A one hundred twenty-second embodiment relates to the recombinant DNA molecule of embodiment 121, wherein said sequence has at least 95 percent sequence identity to the DNA sequence of any of SEQ ID NOs: 1-7, 86-89, 95 and 105.

A one hundred twenty-third embodiment relates to the recombinant DNA molecule of embodiment 121, wherein the protein confers herbicide tolerance in plants.

A one hundred twenty-fourth embodiment relates to the recombinant DNA molecule of embodiment 121, wherein the protein confers pest resistance in plants.

A one hundred twenty-fifth embodiment relates to transgenic plant cell comprising the recombinant DNA molecule of embodiment 121.

A one hundred twenty-sixth embodiment relates to the transgenic plant cell of embodiment 125, wherein said transgenic plant cell is a monocotyledonous plant cell.

A one hundred twenty-seventh embodiment relates to the transgenic plant cell of embodiment 125, wherein said transgenic plant cell is a dicotyledonous plant cell.

A one hundred twenty-eighth embodiment relates to a transgenic seed, wherein the seed comprises the recombinant DNA molecule of embodiment 121.

A one hundred twenty-ninth embodiment relates to a recombinant DNA molecule comprising a plant expressible promoter operably linked to a heterologous nucleic acid sequence encoding a protein, wherein the nucleic acid sequence comprises a recombinant Kozak sequence comprising one or more of an A or G at the −3 position; a G at the +4 position; a C at the −1 position; and a C at the −2 position.

A one hundred thirtieth embodiment relates to a recombinant DNA molecule comprising a plant expressible promoter operably linked to a heterologous nucleic acid sequence encoding a protein, wherein the nucleic acid sequence comprises a recombinant Kozak sequence comprising an C or T at the −3 position and an A, C, or T at the +4 position.

A one hundred thirty-first embodiment relates to a recombinant DNA molecule comprising a plant expressible promoter operably linked to a heterologous nucleic acid sequence encoding a protein, wherein the nucleic acid sequence comprises a recombinant Kozak sequence comprising one or more of a C or T at the −3 position; an A, C or T at the +4 position; an A, G or T at the −1 position; and an A, G or T at the −2 position.

A one hundred thirty-second embodiment relates to a recombinant DNA molecule comprising a plant expressible promoter operably linked to a heterologous nucleic acid sequence encoding a protein, wherein the nucleic acid sequence comprises a recombinant Kozak sequence comprising one or more of an A at the −4 position; an A at the −3 position; an A at the −2 position; an A at the −1 position; a G at the +4 position; and a C at the +5 position.

A one hundred thirty-third embodiment relates to a recombinant DNA molecule comprising a plant expressible promoter operably linked to a heterologous nucleic acid sequence encoding a protein, wherein the nucleic acid sequence comprises a recombinant Kozak sequence comprising one or more of a C, T, or G at the −4 position; a C, T, or G at the −3 position; a C, T, or G at the −2 position; a C, T, or G at the −1 position; an A, C or T at the +4 position; and an A, G or T at the +5 position.

A one hundred thirty-fourth embodiment relates to a recombinant DNA molecule comprising a plant expressible promoter operably linked to a heterologous nucleic acid sequence encoding a protein, wherein the nucleic acid sequence comprises a recombinant Kozak sequence comprising: (a) at least two A's between positions −4 to −1; or (b) one A between positions −4 and −1 and a G at position +4.

A one hundred thirty-fifth embodiment relates to a recombinant DNA molecule comprising a plant expressible promoter operably linked to a heterologous nucleic acid sequence encoding a protein, wherein the nucleic acid sequence comprises a recombinant Kozak sequence comprising less than two A's between positions −4 and −1 and no G at position +4.

A one hundred thirty-sixth embodiment relates to a recombinant DNA molecule comprising a plant expressible promoter operably linked to a heterologous nucleic acid sequence encoding a protein, wherein the nucleic acid sequence comprises a recombinant Kozak sequence comprising one or more of a T at the −8 position, an A or T at the −5 position, a T at the −4 position, a T or C at the −3 position, a T or G at the −2 position, an A, T or C at the +4 position, a G or T at the +5 position, and an A or T at the +6 position.

A one hundred thirty-seventh embodiment relates to a recombinant DNA molecule comprising a plant expressible promoter operably linked to a heterologous nucleic acid sequence encoding a protein, wherein the nucleic acid sequence comprises a recombinant Kozak sequence comprising one or more of a C, G or T at the −6 position, a C, G or T at the −4 position, a C or T at the −3 position, a G or T at the −2 position, a C, G or T at the −1 position, a C, A or T at the +4 position, a G, A or T at the +5 position, and a C or A at the +6 position.

A one hundred thirty-eighth embodiment relates to the recombinant DNA molecule of embodiments 129-137, wherein the nucleic acid molecule encoding the protein encodes one or more N-terminal amino acid modifications of the protein.

A one hundred thirty-ninth embodiment relates to the recombinant DNA molecule of embodiment 138, wherein the one or more N-terminal amino acid modifications introduces an N-terminal sequence selected from the group consisting of: Methionine-Alanine-Serine-Serine wherein Alanine is coded by the codon GCG; Methionine-Alanine-Serine-Serine wherein Alanine is coded by the codon GCT; Methionine-Alanine-Alanine; Methionine-Alanine-Serine-Leucine; and Methionine-Alanine-Alanine-Leucine.

A one hundred fortieth embodiment relates to the recombinant DNA molecule of embodiments 129-139, wherein the protein confers herbicide tolerance in plants.

A one hundred forty-first embodiment relates to the recombinant DNA molecule of embodiments 129-139, wherein the protein confers pest resistance in plants.

A one hundred forty-second embodiment relates to transgenic plant cell comprising the recombinant DNA molecule of embodiments 129-141.

A one hundred forty-third embodiment relates to the transgenic plant cell of embodiment 142, wherein said transgenic plant cell is a monocotyledonous plant cell.

A one hundred forty-fourth embodiment relates to the transgenic plant cell of embodiment 142, wherein said transgenic plant cell is a dicotyledonous plant cell.

A one hundred forty-fifth embodiment relates to a transgenic seed, wherein the seed comprises the recombinant DNA molecule of embodiments 129-141.

A one hundred forty-sixth embodiment relates to a method of identifying features of Kozak sequences conferring high translational efficiency, the method comprising:

determining RNA accumulation and ribosome protection levels for a group of genes expressed in a eukaryotic cell;
selecting genes exhibiting high RNA accumulation and/or ribosome protection levels;
identifying Kozak sequences of the selected genes;
aligning the identified Kozak sequences; and
generating a Kozak consensus sequence.

A one hundred forty-seventh embodiment relates to the method of embodiment 146, wherein genes exhibiting 50 or more Fragments Per Kilobase of transcript per Million (FPKM) are selected.

A one hundred forty-eighth embodiment relates to the method of embodiment 146, wherein genes exhibiting 25 or more Fragments Per Kilobase of transcript per Million (FPKM) are selected.

A one hundred forty-ninth embodiment relates to the method of embodiment 146, wherein at least 25, at least 50, at least 75, at least 100, at least 125, at least 150, at least 175, or at least 200 genes are selected as exhibiting high RNA accumulation and/or ribosome protection levels.

A one hundred fiftieth embodiment relates to the method of embodiment 146, wherein the Kozak sequence comprises nucleotides at positions −9, −8, −7, −6, −5, −4, −3, −2, −1, +4, and +5 where the “A” nucleotide of the ATG start codon is delineated as +1.

A one hundred fifty-first embodiment relates to the method of embodiment 146, further comprising identifying positions within the Kozak sequences of the selected genes that have highly conserved nucleotides.

A one hundred fifty-second embodiment relates to the method of embodiment 146, further comprising identifying poorly represented nucleotides at positions within the Kozak sequences of the selected genes.

A one hundred fifty-third embodiment relates to a method of identifying features of Kozak sequences conferring weak translational efficiency, the method comprising:

determining RNA accumulation and ribosome protection levels for a group of genes expressed in a eukaryotic cell;
selecting genes exhibiting low RNA accumulation and/or ribosome protection levels;
identifying Kozak sequences of the selected genes;
aligning the identified Kozak sequences; and
generating a Kozak consensus sequence.

A one hundred fifty-fourth embodiment relates to the method of embodiment 153, wherein genes exhibiting less than 5 Fragments Per Kilobase of transcript per Million (FPKM) are selected.

A one hundred fifty-fifth embodiment relates to the method of embodiment 153, wherein genes exhibiting less than 1 Fragments Per Kilobase of transcript per Million (FPKM) are selected.

A one hundred fifty-sixth embodiment relates to the method of embodiment 153, wherein at least 25, at least 50, at least 75, at least 100, at least 125, at least 150, at least 175, or at least 200 genes are selected as exhibiting low RNA accumulation and/or ribosome protection levels.

A one hundred fifty-seventh embodiment relates to the method of embodiment 153, wherein the Kozak sequence comprises nucleotides at positions −9, −8, −7, −6, −5, −4, −3, −2, −1, +4, and +5 where the “A” nucleotide of the ATG start codon is delineated as +1.

A one hundred fifty-eighth embodiment relates to the method of embodiment 153, further comprising identifying positions within the Kozak sequences of the selected genes that have highly conserved nucleotides.

A one hundred fifty-ninth embodiment relates to the method of embodiment 153, further comprising identifying poorly represented nucleotides at positions within the Kozak sequences of the selected genes.

The invention may be more readily understood through reference to the following examples, which are provided by way of illustration, and are not intended to be limiting of the invention, unless specified. It should be appreciated by those of skill in the art that the techniques disclosed in the following examples represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

### Example 1. Determination of Consensus Kozak Sequences

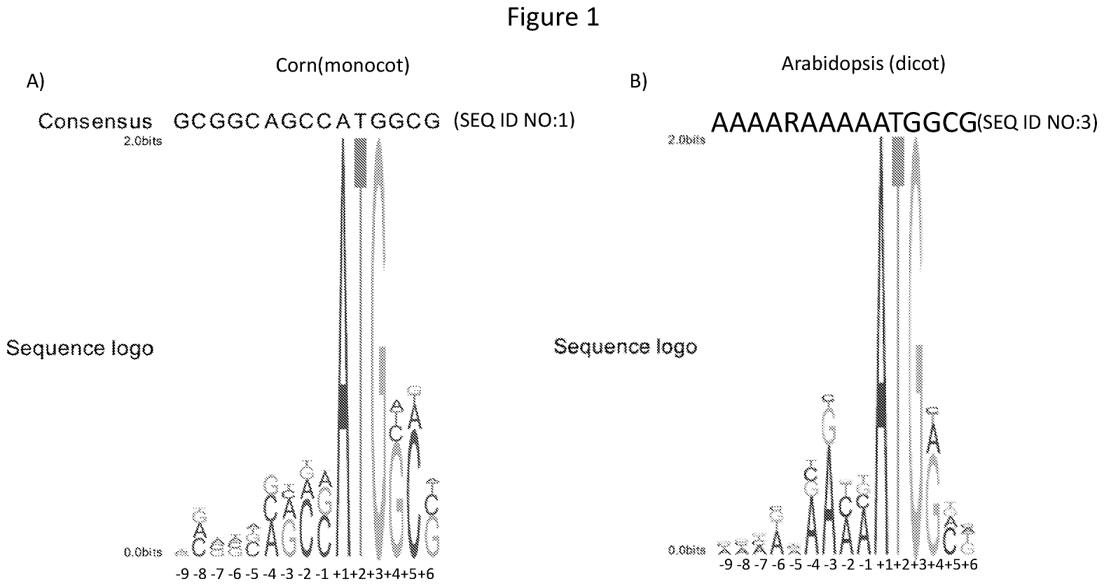

Determining consensus Maize Kozak sequence. Ribo-seq (a high-throughput technique to study global translation (see Hsu et. al. 2016)) and RNA-seq data were generated from maize leaf samples and used as inputs for the program RiboTaper (Calviello et al., 2016). Genes were categorized as low RNA accumulation (5 or fewer Fragments Per Kilobase of transcript per Million (FPKM)) or high RNA accumulation (>50 FPKM). Within each RNA accumulation category, genes were ranked by Open Reading Frames per million (a measurement of ribosome protection), as calculated by RiboTaper. About 100 genes at the top and the bottom of each of these rankings were assembled as classes. After this gene classification by RNA accumulation and ribosome protection levels, the Kozak sequences for the genes within each class were determined and then aligned for sequence logos via CLC Main Workbench (NCBI Resource Coordinators, 2016; Schneider and Stephens, 1990; QIAGEN). 9 bps upstream and 3 bps downstream of the ATG of each gene were included for Kozak sequence alignment. (The A nucleotide of the start codon “ATG” designated as +1 with the preceding base being labeled as −1). A consensus sequence for genes with high translation efficiency was identified (SEQ ID NO:1) from alignment of the Kozak sequences from 99 maize genes with high mRNA expression and high ribosomal protection. See Table 1 and the sequence logo is shown in FIG. 1A.

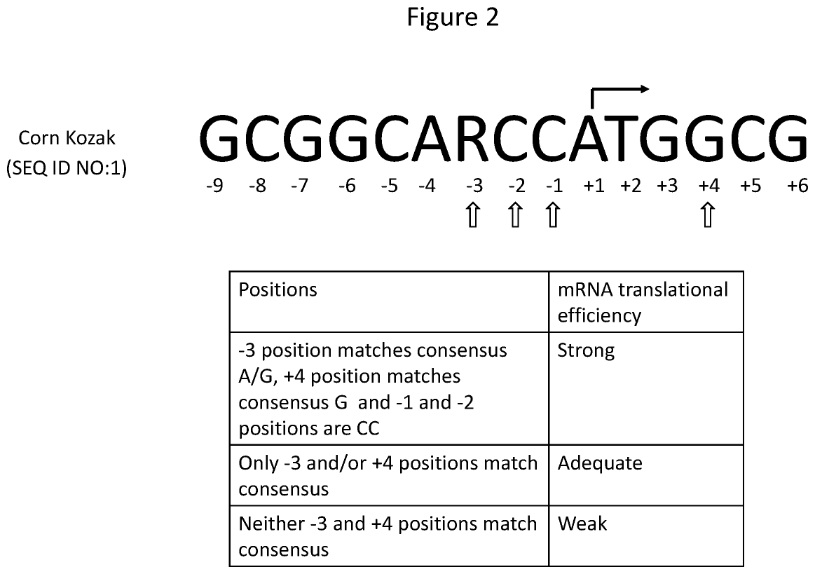

Further analysis of the consensus sequence of ‘strong’ (high translational efficiency) Kozak sequences identified the following features: nucleotides at position −3 that match the consensus G/A (with a slight preference for G); nucleotides at position +4 that match the consensus G; nucleotides at position −1 that match the consensus C and nucleotides at position −2 that match the consensus C. In addition, ‘adequate’ Kozak sequences were found to comprise nucleotides at positions −3 and/or +4 match the consensus sequence, while ‘weak’ Kozak sequences did comprise nucleotides at positions −3 and/or +4 that matched the consensus sequence. See FIG. 2. The Riboseq data was also used to identify nucleotides that were least enriched at each position, and this was used to develop a “depleted” Kozak sequence. See Table 1. Without being bound by any particular theory, inclusion of a depleted Kozak sequence is expected to alter gene expression by reducing mRNA translation efficiency.

Determining consensus Arabidopsis Kozak sequence. A workflow similar to that described above for maize was used to analyze published Arabidopsis (Hsu et. al., 2016) Riboseq datasets, except that high RNA accumulation was defined as >25 FPKM and low RNA accumulation was defined as <1 FPKM. The top 100 genes with high mRNA expression and ribosomal protection were identified and consensus sequences for the strong Kozak and depleted Kozak were determined (see Table 1 and FIG. 1B). Further analysis of the consensus sequence determined the following features of ‘strong’ Arabidopsis Kozak sequences: the nucleotides at positions −4, −3, −2 and −1 comprise A's; the nucleotides at position +4 comprise G; and the nucleotides at position +5 comprise a C. In addition, ‘adequate’ Arabidopsis Kozak sequences comprise at least two A's between positions −4 to −1 OR one A between −4 and −1 and a G at +4. A ‘weak’ Arabidopsis Kozak sequence comprises less than two A's between −4 and −1 positions and no G at position +4.

Determining consensus Tomato Kozak sequence. Published Riboseq and RNAseq data in tomato was used for this analysis (Wu et al, 2019). Genes were classified based on expression level; High (>25 FPKM), Intermediate (1-25 FPKM) and Low (<1 FPKM). Genes were then sorted by translational efficiency. 100 tomato genes with high mRNA expression and high translation efficiency were selected. 9 bps upstream and 3 bps downstream of the ATG of each gene were included for Kozak sequence alignment. The consensus sequence for the Tomato strong Kozak and depleted Kozak is shown in Table 1.

TABLE 1
Plant Kozak consensus sequences. Underlined nucleotides indicate
the start. R = A or G. N = A, T, G or C

| | Strong Kozak Consensus | |
|---|---|---|
| Organism | sequence (5′ to 3′) | Depleted Kozak sequence |
| Maize | GCGGCARCCATGGCG | GTTTATTTTATGAGA |
| | (SEQ ID NO 1) | (SEQ ID NO 2) |
| Arabidopsis | AAAARAAAAATGGCG | GGGCTTCGTATGCTC |
| | (SEQ ID NO 3) | (SEQ ID NO 4) |
| Tomato | TTAACAAAAATGGCT | CNGCGCCGTATGCGC |
| | (SEQ ID NO 5) | (SEQ ID NO 6) |
| Rice | C(G/C)R(A/C)(G/C)ATGGCG | — |
| (Rangan et al., 2008) | (SEQ ID NO 7) | |

### Example 2. Editing Native Kozak Sequences to Fine Tune Protein Expression

Based on the sequence information described in Example 1 the inventors devised a methodology to selectively modify mRNA translation and protein accumulation by introducing point mutations within the Kozak sequence of endogenous genes. For selected maize proteins, a desired expression strategy (e.g., up-regulation or down-regulation of expression of the selected protein) is chosen and the native Kozak sequence of the gene encoding the selected protein is identified. The native Kozak sequence is then aligned to the maize consensus sequence for ‘strong’ (high translational efficiency) genes (SEQ ID NO. 1) and the relative strength (strong, adequate, weak) of the native Kozak sequence is determined by comparing the native Kozak sequence to features identified as indicative of strong, adequate or weak mRNA translational efficiency. See FIG. 2. In the event the native Kozak sequence does not comprise features indicative of strong mRNA translational efficiency (e.g., an A or G at the −3 position, G at the +4 position, C at the −1 position, and C at the −2 position) and increased accumulation of the selected protein is desired, gene editing is employed to introduce edits so as to change the native sequence from a “weak” state to the “adequate” or “strong” state, or from the “adequate” state to the “strong” state. In the event the Kozak sequence comprises features indicative of strong or adequate mRNA translational efficiency and downregulation of the selected protein is desired, gene editing is used to change the native sequence from the “strong” state to the “adequate”/“weak” state, or from the “adequate” to the “weak” state (e.g., changing A or G at the −3 position to C or T, and/or G at the +4 position to C, T or A, and/or C at the −1 position to G, T or A, and/or C at the −2 position to G, T or A). To significantly downregulate protein expression, precise mutations can be introduced to convert a native Kozak to the ‘depleted’ maize Kozak sequence of SEQ ID NO. 2.

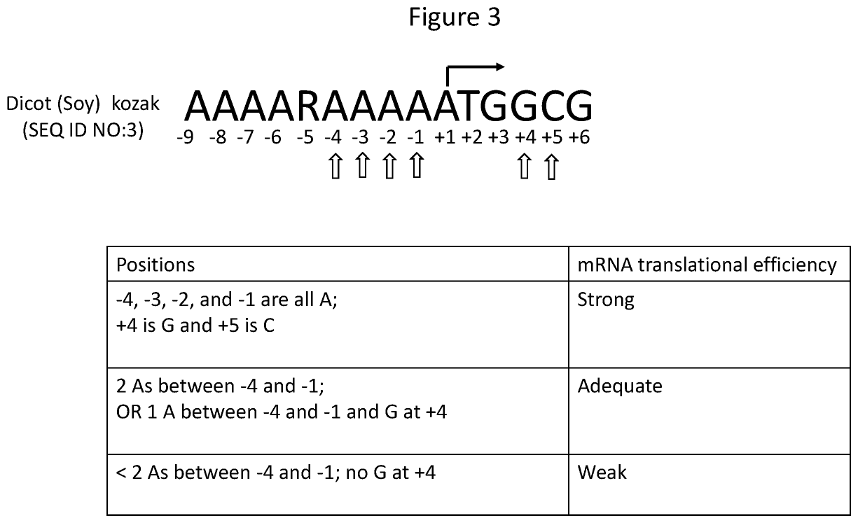

Selective modification of mRNA translation and protein accumulation in soybean plants is achieved by introducing point mutations within the Kozak sequence of endogenous soy genes. For selected soy proteins, a desired expression strategy (e.g., up-regulation or down-regulation of expression of the selected soy protein) is chosen and the native Kozak sequence of the gene encoding the selected protein is identified. The native Kozak sequence is then aligned to the consensus sequence for ‘strong’ (high translational efficiency) dicot genes (SEQ ID NO. 3) and the relative strength (strong, adequate, weak) of the native Kozak sequence is determined by comparing the native Kozak sequence to features identified as indicative of strong, adequate or weak mRNA translational efficiency. See FIG. 3. In the event the native Kozak sequence does not comprise features indicative of strong mRNA translational efficiency (e.g., an A at the −4 position, an A at the −3 position, an A at the −2 position, an A at the −1 position, a G at the +4 position, and a C at the +5 position) and increased accumulation of the selected protein is desired, gene editing is employed to change the native sequence from the “weak” state to the “adequate”/“strong” state, or from the “adequate” state to the “strong” state. In the event the Kozak sequence comprises features indicative of strong or adequate mRNA translational efficiency and downregulation of the selected soy protein is desired, gene editing is used to change the native sequence from the “strong” state to the “adequate” or “weak” state, or from the “adequate” to the “weak” state (e.g., changing an A at the −4 position to T, C or G, an A at the −3 position to T, C or G, an A at the −2 position to T, C or G, an A at the −1 position to T, C or G, a G at the +4 position to C, T, or A, and/or a C at the +5 position to G, T, or A). To significantly downregulate soy protein expression, precise mutations can be introduced to convert a native Kozak to the ‘depleted’ dicot Kozak sequence of SEQ ID NO. 4.

### Example 3: Editing Kozak Sequences of Maize and Soy Target Genes

Five maize genes and two soy genes are chosen to test if targeted manipulations of Kozak sequences result in modification of protein expression. The Waxy gene of maize has a recognizable phenotype and has been broadly used in classical and molecular genetics as a model gene (see Shure et al., 1983). Agronomically, Waxy maize exhibits better feed gain than conventional maize (see Camp et al., 2003). Maize Brown Midrib (BM3) frameshift mutants have reduced lignin content and thus improved cell wall digestibility (see Jung et al., 2012). Rad54 and Ku70 genes are involved in DNA repair and recombination (see Kragelund et al., 2016; Mazin et al., 2010). Modification of the expression of these genes can offer some control over meiotic recombination or other DNA repair processes in cells. Rpt is a tandem duplicated disease resistance locus in maize against maize rust (see Smith et al., 2004). Manipulating expression of these genes can offer more control over disease resistance responses in maize. The Rpt paralog shown in these examples have two tandem genomic copies in the maize genome. Altering expression for not just one, but two related genes at a time can have a larger effect on overall expression and phenotype than doing so for a single-copy gene.

The lipoxygenase (LOX) gene of soy is a key element of fatty acid metabolism and such, has a direct influence on the quality of food and feed (Eskin et al., 1977; Lenis et al., 2010). The alpha-SNAP protein of soy is involved in intracellular transport and is implicated with soy cyst nematode resistance (Butler et al., 2019). Similar to the Rp1 gene in maize, alpha-SNAP has three identical copies in the W82 public reference genome of soy. Manipulation of the Kozak sequences of multiple gene copies can broaden the dynamic range of gene expression. The genomic regions surrounding the Kozak sequences of these genes and their predicted mRNA translational efficiency (strong, adequate, weak) are shown Table 2. Genomic sequences around the Kozak sites of the 7 genes were analyzed to identify Cas12a and/or Cas9 CRISPR targets sites (See Tables 3 and 4). Three Cas12a enzymes, differing in their protospacer adjacent motif (PAM) recognitions, are considered: LbCas12a that recognizes the PAM sequence TTTV); a variant LbCas12a-RR that comprises the mutations G532R/K595R and recognizes the PAM sequence 5-TYCV and FnCas12a that recognizes the TTV PAM sequence.

TABLE 2
Maize and Soy Target genes. The SEQ ID NOs represent genomic
fragments of the target gene comprising the Kozak sequence, region
of the 5′UTR and region of exon 1 comprising the start site.

| | Predicted Native (WT) Kozak | |
|---|---|---|
| Target gene | mRNA translational efficiency | SEQ ID NO |
| ZmWaxy | Strong | 8 |
| ZmBm3 | Strong | 9 |
| ZmRad54 | Weak | 10 |
| ZmRp1 | Adequate | 11 |
| ZmKu70 | Strong | 12 |
| GmLox | Adequate | 13 |
| GmSNAP | Adequate | 14 |

TABLE 3
List of representative Cas12a CRISPR target sites at or near the Kozak
sequences of five maize (Zm) and two soy (Gm) genes

| | | | Target site sequence | | |
|---|---|---|---|---|---|
| Gene | Enzyme | Target siteName | PAM | Spacer (23nt) | SEQ ID NO |
| ZmWaxy1 | FnCas12a | ZmWaxy1_FnCas12a_TS1 | TTA | ATCGGCATGGCGGCTCTAGCCAC | 29 |
| ZmWaxy1 | FnCas12a | ZmWaxy1_FnCas12a_TS2 | TTG | CGACGAGCTGCGACGTGGCTAGA | 30 |
| ZmRp1 | FnCas12a | ZmRp1_FnCas12a_TS1 | TTC | ATGGCGGACTTGGCGCTAGTTGG | 31 |
| ZmRp1 | FnCas12a | ZmRp1_FnCas12a_TS2 | TTA | AGCCAACTAGCGCCAAGTCCGCC | 32 |
| ZmRp1 | LbCas12a-RR | ZmRp1_LbCas12a-RR_TS1 | TCCA | ACTTCATGGCGGACTTGGCGCTA | 33 |
| ZmRp1 | LbCas12a-RR | ZmRp1_LbCas12a-RR_TS2 | TTCA | TGGCGGACTTGGCGCTAGTTGGC | 34 |
| ZmRp1 | LbCas12a-RR | ZmRp1_LbCas12a-RR_TS3 | TCCG | CCATGAAGTTGGAGTAGTTTGTG | 35 |
| ZmKu70 | FnCas12a | ZmKu70_FnCas12a_TS1 | TTC | CCGACCTCGGCGCCATGGACCTG | 36 |
| ZmKu70 | LbCas12a-RR | ZmKu71_LbCas12a-RR_TS1 | TCCA | GTTCCCGACCTCGGCGCCATGGA | 37 |
| ZmKu70 | LbCas12a-RR | ZmKu71_LbCas12a-RR_TS2 | TTCC | CGACCTCGGCGCCATGGACCTGG | 38 |
| ZmKu70 | LbCas12a-RR | ZmKu71_LbCas12a-RR_TS3 | TCCC | GACCTCGGCGCCATGGACCTGGA | 39 |
| ZmKu70 | LbCas12a-RR | ZmKu71_LbCas12a-RR_TS4 | TCCA | TGGCGCCGAGGTCGGGAACTGGA | 40 |
| ZmKu70 | LbCas12a-RR | ZmKu71_LbCas12a-RR_TS5 | TCCA | GGTCCATGGCGCCGAGGTCGGGA | 41 |
| ZmKu70 | LbCas12a-RR | ZmKu71_LbCas12a-RR_TS6 | CCCC | TCTGGGTCCAGGTCCATGGCGCC | 42 |
| ZmKu70 | LbCas12a-RR | ZmKu71_LbCas12a-RR_TS7 | TCCC | CTCTGGGTCCAGGTCCATGGCGC | 43 |
| ZmRad54 | FnCas12a | ZmRad54_FnCas12a_TS1 | TTA | TTCACCGTCCGTTGCAGCGAATG | 44 |
| ZmRad54 | FnCas12a | ZmRad54_FnCas12a_TS2 | TTC | ACCGTCCGTTGCAGCGAATGCCC | 45 |
| ZmRad54 | FnCas12a | ZmRad54_FnCas12a_TS3 | TTG | CAGCGAATGCCCTCGAGGAGCCA | 46 |
| ZmRad54 | LbCas12a | ZmRad54_LbCas12a_TS1 | TTTA | TTCACCGTCCGTTGCAGCGAATG | 47 |
| ZmRad54 | LbCas12a-RR | ZmRad54_LbCas12a-RR_TS1 | TTCA | CCGTCCGTTGCAGCGAATGCCCT | 48 |
| ZmRad54 | Cas12a-RR | ZmRad54_LbCas12a-RR_TS2 | TCCG | TTGCAGCGAATGCCCTCGAGGAG | 49 |
| Soy genes | | | | | |
| GmLOX | FnCas12a | GmLOX_FnCas12a_TS1 | TTG | GCAAAGATGTTTTCAGCAGGCCA | 51 |
| GmLOX | FnCas12a | GmLOX_FnCas12a_TS2 | TTG | TGTTGGTAGCTTTGGCAAAGATG | 52 |
| GmLOX | FnCas12a | GmLOX_FnCas12a_TS3 | TTG | GTAGCTTTGGCAAAGATGTTTTC | 53 |
| GmLOX | FnCas12a | GmLOX_FnCas12a_TS4 | TTG | CCAAAGCTACCAACACAACTATT | 54 |
| GmLOX | LbCas12a | GmLOX_LbCas12a_TS1 | TTTG | GCAAAGATGTTTTCAGCAGGCCA | 55 |
| GmLOX | LbCas12a | GmLOX_LbCas12a_TS2 | TTTG | CCAAAGCTACCAACACAACTATT | 56 |
| GmLOX | LbCas12a | GmLOX_LbCas12a_TS3 | TTTG | ATCTTATGGCCTGCTGAAAACAT | 57 |
| GmLOX | LbCas12a-RR | GmLOX_LbCas12a-RR_TS1 | TCCC | TTTGATCTTATGGCCTGCTGAAA | 58 |
| GmSNAP | FnCas12a | GmSNAP_FnCas12a_TS1 | TTC | GATCGGAGGAAAATGGCCGATCA | 59 |
| GmSNAP | FnCas12a | GmSNAP_FnCas12a_TS2 | TTG | TTTCGATCGGAGGAAAATGGCCG | 60 |
| GmSNAP | FnCas12a | GmSNAP_FnCas12a_TS3 | TTC | GATAACTGATCGGCCATTTTCCT | 61 |
| GmSNAP | LbCas12a | GmSNAP_LbCas12a_TS1 | TTTC | GATCGGAGGAAAATGGCCGATCA | 62 |
| GmSNAP | LbCas12a | GmSNAP_LbCas12a_TS2 | TTTG | TTTCGATCGGAGGAAAATGGCCG | 63 |
| GmSNAP | LbCas12a- | GmSNAP_LbCas12a-RR_TS1 | TTCG | ATCGGAGGAAAATGGCCGATCAG | 64 |
| GmSNAP | LbCas12a- | GmSNAP_LbCas12a-RR_TS2 | TTCG | ATAACTGATCGGCCATTTTCCTC | 65 |

TABLE 4
List of representative Cas9 CRISPR target sites at or near the
Kozak sequences of maize and soy genes

| | | | Target site sequence | | |
|---|---|---|---|---|---|
| Gene | Enzyme | TS name | Spacer | PAM | SEQ ID NO: |
| ZmBM3 | Cas9 | ZmBM3_Cas9_TS1 | GTCGCCGGCGGTGGAGCCCA | TGG | 50 |
| GmSNAP | Cas9 | GmSNAP_Cas9_TS1 | TTGTTTCGATCGGAGGAAAA | TGG | 66 |
| GmSNAP | Cas9 | GmSNAP_Cas9_TS2 | AATTGCTTTGTTTCGATCGG | AGG | 67 |

### Example 4: Molecular Constructs and Plant Transformation Methods Used for Delivering Editing Reagents

Genome editing reagents can be delivered into the host plants using DNA expression vectors optimized for expression in the host plant. Delivery methods of DNA-based molecular constructs include but are not limited to (1) polyethylene-glycol (PEG) mediated protoplast transformation, (2) Agrobacterium-mediated transformation, (3) particle bombardment and (4) carbon nanoparticle delivery.

In Agrobacterium-mediated plant transformation (Agro transformation) the Type IV secretion system of the plant pathogens Agrobacterium tumefaciens or Rhizobium (formerly Agrobacterium rhizogenes) is engineered such that exogenous plasmid DNA (T-DNA) transformed into Agrobacterium would ultimately integrate into the plant host genome by a well-defined molecular machinery. Due to its broad adaptability to multiple species and scalability, this method is the most prevalent one in plant transformation. Agrobacterium T-DNA vectors are designed for delivery of CRISPR nuclease system components to plant cells. CRISPR nuclease is encoded by an individual expression cassette, which is assembled in a single T-DNA molecule in a binary vector suitable for use with Agrobacterium tumefaciens strains. The T-DNA vector is further designed to contain an expression cassette for production of at least one suitable gRNA that forms a complex with Cas12a or Cas9 and guides it to hybridize to a target site in a plant genome. An expression cassette for a plant selectable marker gene, for example antibiotic resistance or herbicide tolerance, is further provided in the T-DNA vectors to aid in selection of transformed plant cells. For editing methodology that require a donor/repair template (see Example 5), the donor/repair template sequence may be incorporated into the expression vector or delivered separately.

Gene expression regulatory elements, including, but not limited to, promoters, introns, polyadenylation sequences and transcriptional termination sequences, are chosen to provide suitable expression levels of each expression element on the T-DNA. Gene expression elements that express the gene cassettes at sufficient levels and timing so as to provide all necessary components at the same time and in the same tissue, at levels that are sufficient to result in targeted cleavage activity are utilized. Promoters and other regulatory elements may be chosen to provide constitutive gene expression of all the components of the system.

The Cas12a guide RNA expression cassette comprises a plant Pol III promoter operably linked to a 21-nucleotide DNA sequence encoding either the FnCas12a crRNA sequence, also called a direct repeat sequence (SEQ ID NO: 70) or an LbCas12a direct repeat sequence (SEQ ID NO: 169); a 23- to 25-nucleotide spacer DNA sequence (SEQ ID NO: 29-49 for maize, SEQ ID NO: 51-65 for soy) targeting one of the 7 genes described in Table 2 followed by a DNA sequence encoding the 19-nucleotide crRNA (SEQ ID NO: 70) and a T7 termination sequence. The Cas9 gRNA expression cassette comprises a PolIII promoter operably linked to a spacer sequence targeting one of the target genes described in Table 2 (SEQ ID NO: 50, 66, 67) operably linked to a 76-nucleotide DNA sequence encoding the Cas9 single guide RNA (sgRNA) (SEQ ID NO: 71) sequence comprising a crRNA and a tracrRNA.

The editing components can also be delivered as ribonucleo-protein (RNP) complexes that are assembled in vitro, prior to transformation. Yet, in another embodiment, they can be delivered as an RNA molecule. It may include the messenger RNA (mRNA) for the effector CRISPR nuclease protein, and, chimerically linked to it, the non-coding RNA for the crRNA/tracrRNA or sgRNA, whichever may apply for the specific experiment. Alternatively, a mix of a separate mRNA and one or more non-coding RNA species can also be delivered. While Cas12a is used as an example, these designs are also suitable for delivering most other effector proteins known in the art including, but not limited to Cas9, Cas12b, Cas12k, Cas13; or fusion derivatives of these used in base editing (BE), prime editing (PE) or in DNA tethering constructs such as Cas:HUH or Cas:streptavidin. In addition to the native Cas effector proteins, amino-acid sequence variants recognizing alternative protospacer-adjacent motifs (PAMs) can also be expressed as needed. While there are many such variants known in the art, Example 7 highlights one particular example: LbCas12a-RR, which carries two, a G/R and a K/R substitutions. This variant recognizes TYCV and CCCC PAMs as oppose to the canonical TTTV PAMs (Gao et al., 2017; Zhong et al., 2018). Table 3 shows examples of Cas9, Cas12a and Cas12a-RR target sites in the genes of interest listed in Table 2.

In protoplast transformation, plant cell walls are removed by an appropriate enzyme mixture (including cellulase, pectinase and xylanase). Then, the cells are suspended in a solution including the plasmid of interest, PEG and calcium cations. The calcium ions, in the presence of PEG form pores in the cell membrane that facilitates the plasmid uptake. This transformation method is considered one of the most efficient one as far as the plasmid/cell ratio is concerned. In a few plant species, whole plants can be regenerated from transformant protoplasts. In others, protoplast transformation is considered rather an experimental model to test heterologous gene expression prior to using alternative stable, plant-based transformation methods.

In particle bombardment, a gold particle coated with the plasmid of interest is delivered into plant tissues in a disruptive manner. Once the gold particles are submerged into the partially damaged tissues, the plasmids can be dissolved into the cytosols. Carbon nanoparticle transformation is the newest of all these technologies. The chemically inert carbon nanoparticles are first covalently coated by a positively charged polymer, such as polyethyleneimine (PEI). Then, these electrostatically active nanoparticles are incubated with the negatively charged DNA, RNA or RNP, which thus will be absorbed by them. Next, these nanoparticle complexes are delivered into plants by a suitable method, such as leaf infiltration or microinjection.

Any of the plant transformation strategies listed above can be viable options for experiments that aimed to edit Kozak sequences in plants.

### Example 5: Editing Kozak Sequences Using Homology-Directed Templated Repair

CRISPR-mediated chromosome cutting at or around the Kozak sequence can trigger homology-directed repair in the presence of an appropriate template. These templates can be used to engineer the Kozak sequence of a gene encoding a protein of interest, thereby modifying protein expression. For each targeted Kozak sequence, repair templates comprising mutations in the −4, −3, −2, −1, +4 and/or +5 positions of the native Kozak sequence are designed and used for homology-directed repair following Cas mediated cleavage at the target region.

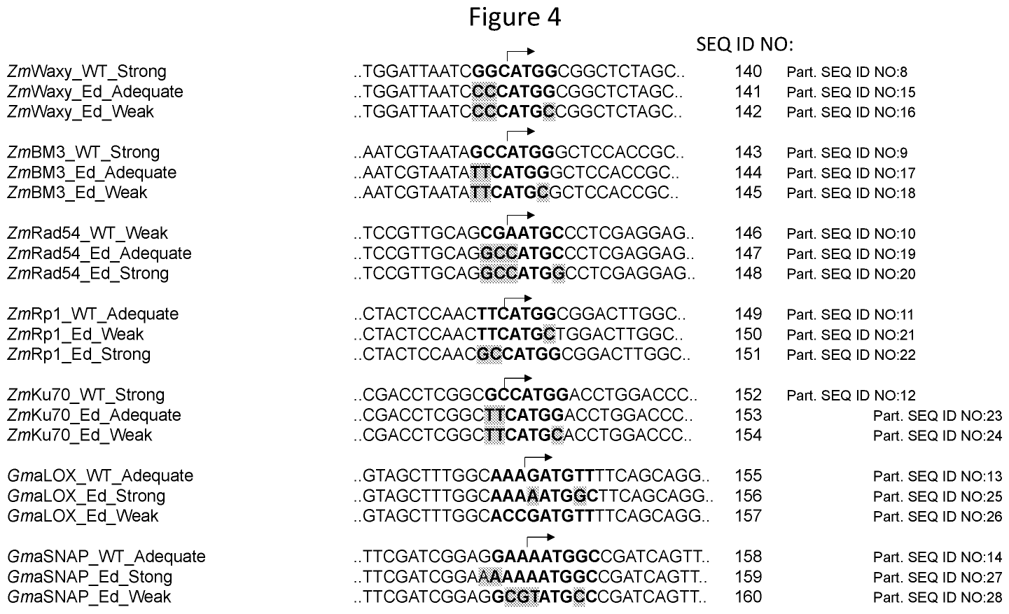

Examples of possible repair templates with optimized Kozak sequences for the 7 target genes are shown in FIG. 4. All these templates are shown in uniform length and in sense orientation. However, their lengths, strandedness (ss/ds) and orientation can vary based on experimental conditions. For example, in at least some eukaryotic organisms, ssDNA templates are preferred to be in the same orientation as the target site. However, the preference for template orientation is not fully established in either soy or maize.

The templates can be incorporated into a binary plasmid designed for Agrobacterium-mediated transformation. In this scenario, the template will be double-stranded, while its length can still be variable. When using either PEG transformation or particle bombardment, single stranded or double stranded templates are optional.

### Example 6: Editing Kozak Sequences by Screening for Targeted Point Mutations, Such as Insertions or Deletions (Indels)

Single or multiple nucleotide insertions or deletions caused by targeted double-strand breaks and subsequent erroneous DNA repairs, if impacting one of the conserved nucleotides of a Kozak sequence can modify mRNA translational efficiency. If a cognate target site of a CRISPR endonuclease, such as Cas9 or Cas12a overlaps with the Kozak sequence of a gene encoding a protein of interest such that the targeted double-strand break (referred to as ‘cut site’ below) coincides or flanks one or more of the nucleotides of the Kozak sequence, it is feasible to screen for indels in the edited plants to identify ones where the Kozak sequence has been modified due to an indel.

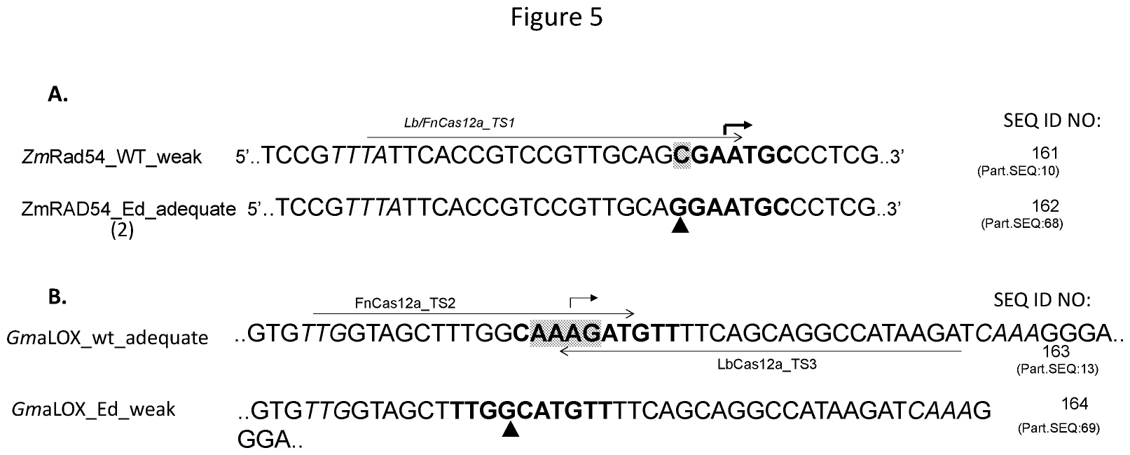

FIG. 5A illustrates an example, where the weak native Kozak sequence of ZmRad54 may be turned to an adequate Kozak sequence by identifying edits comprising the deletion of a ‘C’ in the −3 position, thus sliding a flanking ‘G’ into the same position. Similarly, FIG. 5B shows how the wild type, adequate Kozak sequence of the GmLOX gene may be converted to a weak Kozak sequence in edits comprising a 4-bp (‘AAAG’) targeted deletion at positions −4 to −1 mediated by either Fn- or LbCas12a.

### Example 7: Editing Kozak Sequences by Base Editing (BE)

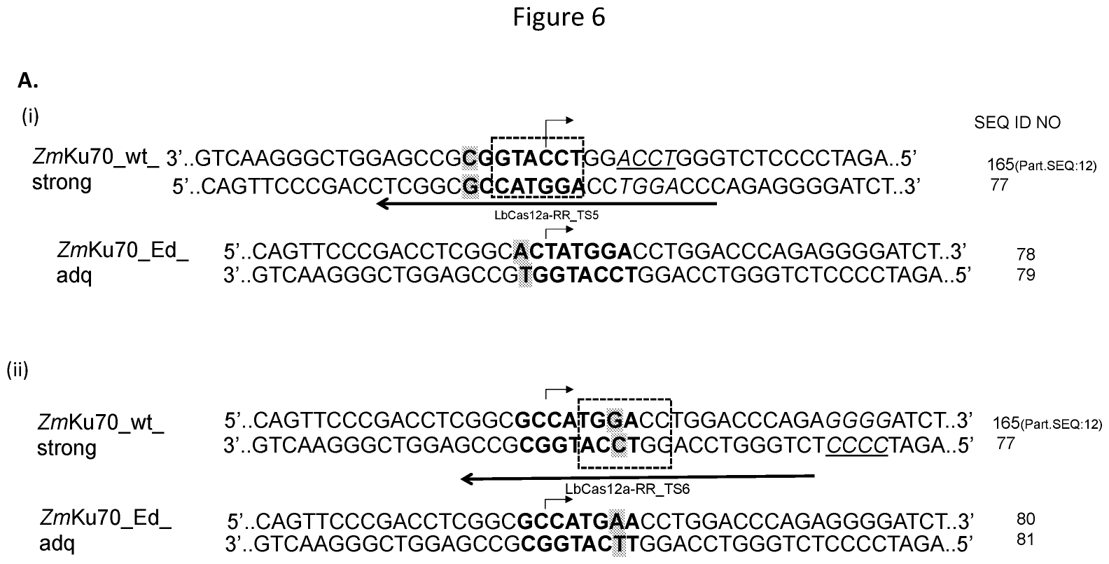

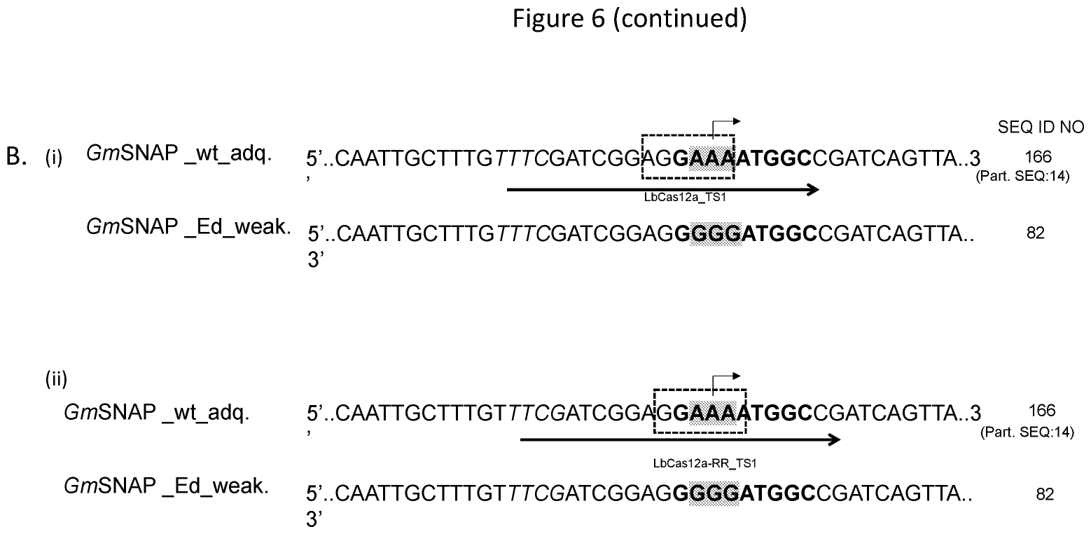

Cytosine base editors (CBEs) are comprised of a single-stranded cytidine deaminase fused to an impaired form of Cas9 or Cas12a, which, at the other terminus is also tethered to one (BE3) or two (BE4) monomers of uracil glycosylase inhibitor (UGI) (Komor et al., 2016 and 2017). CBEs catalyze C-to-T conversions. Adenine base editors (ABEs) include deoxyadenosine deaminases, which catalyze conversions of adenosines to inosines. Inosines are read as guanines by polymerases, which thus ultimately convert As to Gs (Gaudelli et al., 2017). Since both deaminases use ssDNA as substrate, nucleotides in only the most exposed portions of the single-stranded R-loops are accessible for such base conversion. More specifically, for Cas12a BEs, conversion rates are the best in the 8-14 bp region downstream of PAM. FIG. 6 shows two examples of how the Kozak sequences of ZmKu70 and GmSNAP may be altered using CBE and ABE, respectively. In both cases, the Kozak sequences overlap with the 8-14 bp region of corresponding target sites.

### Example 8: Editing Kozak Sequences by Prime Editing (PE)

Prime editing is a genome editing technology that can introduce selected mutations at or around the nick site of a CRISPR nickase (Anzalone et al., 2019). Prime editing has been described as a ‘search-and-replace’ genome editing technology that mediates targeted insertions, deletions, all 12 possible base-to-base conversions, and combinations thereof without requiring double stranded breaks (DSBs) or donor DNA templates. Prime editors are fusion proteins between a CRISPR-associated nickase (e.g., Cas9, Cas12a) and an engineered reverse transcriptase. The prime editor protein is targeted to the editing site by an engineered prime editing guide RNA (pegRNA). pegRNAs have dual functions: they guide the prime editor to the specified target site and encode the desired edit in an extension that is typically at the 3′ end of the pegRNA. Upon target binding, the CRISPR nickase introduces a single strand break in the PAM-containing DNA strand. The prime editor then uses the newly liberated 3′ end of the target DNA site to prime reverse transcription using the extension in the pegRNA as a template. Successful priming requires that the extension in the pegRNA contain a primer binding sequence (PBS) that can hybridize with the 3′end of the nicked target DNA strand to form a primer-template complex. In addition, pegRNAs contain a reverse transcription template that directs the synthesis of the edited DNA strand onto the 3′end of the target DNA strand. The reverse transcription template contains the desired DNA sequence change(s), as well as a region of homology to the target site to facilitate DNA repair.

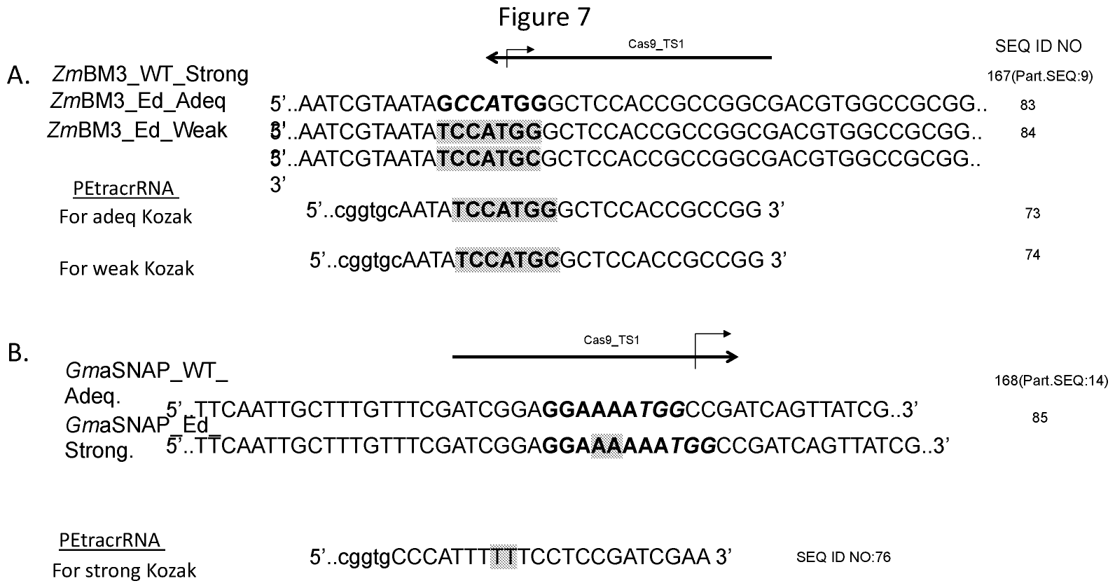

FIG. 7 illustrates how the native Kozak regions of ZmBM3 (strong Kozak) and GmSNAP (adequate Kozak) can be altered by prime editing. Since prime editing can function using separate crRNA and prime-edit-modified tracrRNAs (petracrRNA), the embodiment described in FIG. 7 utilizes separate crRNA and petracrRNAs. The ZmBM3 Cas9 TS1 crRNA sequence is set forth as SED ID NO: 72. The petracrRNA of SEQ ID NO: 73 is designed as a template for converting the native strong Kozak of BM3 (SEQ ID NO:167) to an adequate Kozak (SEQ ID NO: 83). The petracrRNA of SEQ ID NO: 74 is designed for converting the native strong Kozak of BM3 (SEQ ID NO:167) to a weak Kozak (SEQ ID NO: 84).

The native GmSNAP gene has an adequate Kozak. The GmSNAP Cas9-TS1 crRNA sequence is set forth as SEQ ID NO: 75. The petracrRNA (SEQ ID NO: 76) is designed for converting the native adequate Kozak of GmSNAP (SEQ ID NO: 85) to a strong Kozak. In another embodiment, a chimeric fused pegRNA is used for prime editing.

### Example 9: Molecular Characterization of Edited Plants

Maize or Soy excised embryos or explants are transformed with a transformation vector having one of the editing constructs described in Example 4. As a control, transformation vectors lacking gRNA cassettes are also transformed. The transformed embryos or explants are transferred to soil plugs for rooting. To characterize the edits and recover plants with relevant edits, DNA is extracted from leaf tissue and PCR-based assays are performed using a pair of PCR primers flanking the intended target region comprising the Kozak sequence region. PCR products are sequenced and analyzed to identify relevant edits. Plants comprising the relevant Kozak edits are grown to maturity and self-pollinated to obtain plants homozygous for the edited allele. The mRNA and protein expression in leaf tissue from edited and control plants are compared. qRT-PCR or RNAseq analysis is used for assessing mRNA expression levels and Western blotting or ELISA is used for assessing protein accumulation. Ribosome profiling followed by Ribo-seq (also called as Ribosome foot printing) can also be used to quantify ribosome occupancy which correlates with protein accumulation. The relative protein expression of the edited alleles compared to the unedited, native allele, is increased for the edited alleles having features of the strong Kozak consensus sequence. Conversely, the protein expression is decreased for the edited alleles lacking features of the strong Kozak consensus sequence (e.g., having features of a depleted Kozak sequence). Edited plants showing desired variations in the protein level are advanced for phenotypic assays relevant for each trait.

### Example 10: Optimizing Transgene Protein Expression by Designing Optimal Sequences Around the Transcription Start Site

This example describes the testing of Kozak sequence variants and N-terminal amino acid modifications and their impact on RNA expression and protein accumulation of 4 proteins of interest. Specifically, selected nucleotide sequences (−9 up to +12) flanking the translation initiator codon (ATG) of transgenes encoding the protein of interest were synthesized and introduced into transgene expression cassettes to test for its effect on mRNA translation efficiency and protein accumulation in protoplasts and in plants.

Target genes and modifications: Gene of Interest 1 (GOI 1) encoding Protein of Interest 1 (POI 1); Gene of Interest 2 (GOI 2) encoding Protein of Interest 1 (POI 2); Gene of Interest 3 (GOI3) encoding Protein of Interest 3 (POI 3) and Gene of Interest 4 (GOI 4) encoding Protein of Interest 4 (POI 4) were selected for this analysis. Four variants of Kozak sequences and nine N-terminal amino acid modifications were selected for testing (see Table 5). The “strong” maize consensus Kozak sequence (SEQ ID NO:1) (described in Table 5 as “Strong-1”) developed by alignment of 99 maize genes with high mRNA expression and high ribosomal protection indicative of high translation efficiency (see Example 1) was selected for testing. Additionally, a second ‘strong’ maize consensus Kozak sequence (SEQ ID NO: 86) (described in Table 5 as “Strong-2”) developed by alignment of 100 maize genes with low mRNA expression and high ribosomal protection and a ‘depleted’ maize Kozak sequence (SEQ ID NO: 2) (described in Table 5 as “Depleted”) were selected for testing.

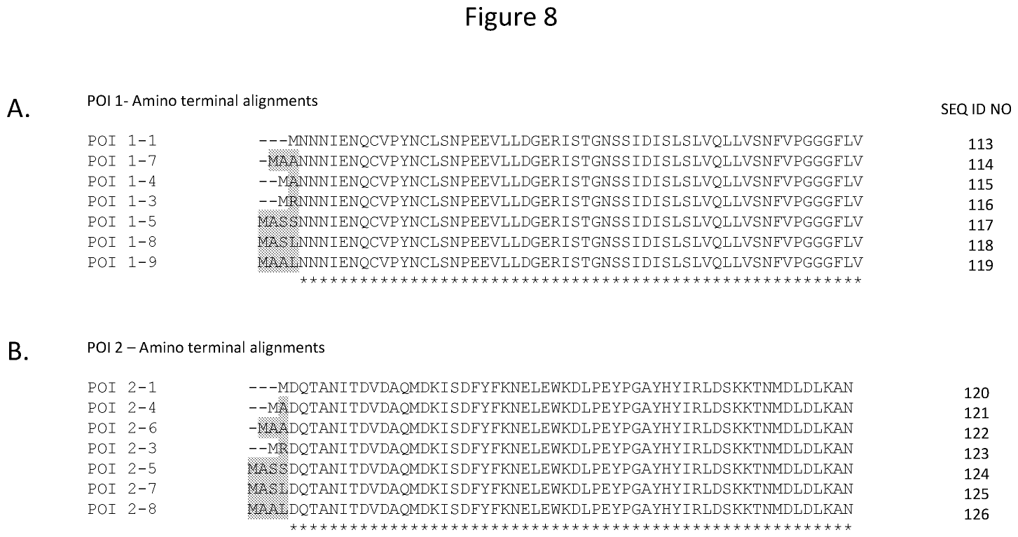

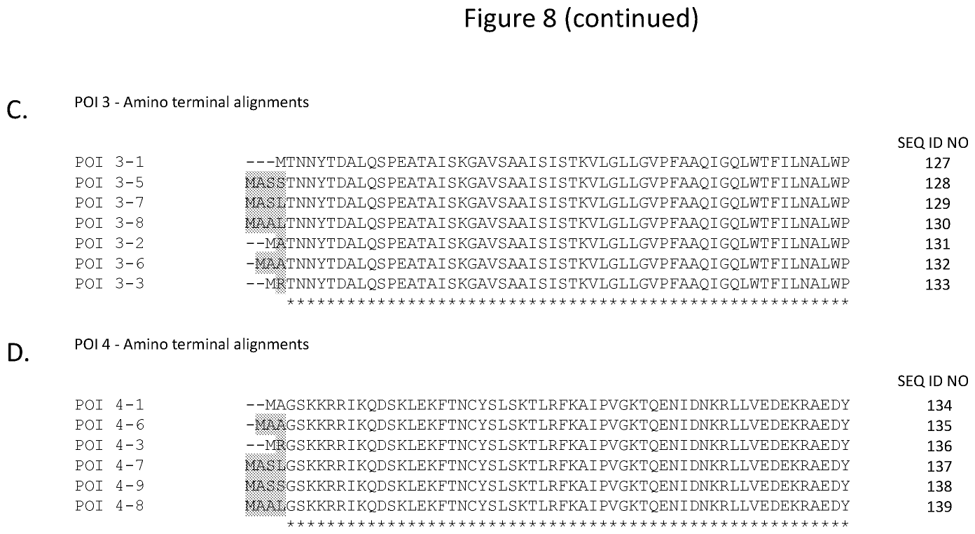

Expression Constructs: Multiple Agrobacterium T-DNA expression constructs comprising gene expression cassettes for each of the four genes comprising corresponding Kozak variant and N-terminal modifications were generated (see Table 5, FIG. 8). Each gene expression cassette comprised the gene encoding the protein of interest with Kozak and/or N-terminal modifications, operably linked to 5′ and 3′ untranslated regions and a plant-operable promoter and leader.

TABLE 5
Construct identities, genes and description of modifications. Original = Native
N-terminal sequence. MASS₁ = Methionine-Alanine-Serine-Serine wherein Alanine is
coded by the codon GCG. MASS₂ = Methionine-Alanine-Serine-Serine wherein Alanine
is coded by the codon GCT. MAA = Methionine-Alanine-Alanine. MASL = Methionine-
Alanine-Serine-Leucine. MAAL = Methionine-Alanine-Alanine-Leucine. * Indicates the
constructs comprising the unoptimized Kozak sequence and original N-terminal
sequence for the specified gene.

| | | | | | Kozak |
|---|---|---|---|---|---|
| Expression | Gene of | Kozak | N-terminal | | SEQ |
| Construct | Interest | Modification | Modification | Sequence around ATG (5′ to 3′) | ID NO |
| POI 1-1* | GOI 1 | Adequate | Original | CTTACCACCATGAAC | 87 |
| POI 1-2 | GOI 1 | Strong-1 | Bonus Ala | GCGGCAGCCATGGCG | 88 |
| POI 1-3 | GOI 1 | Depleted | Bonus Arg | GTTTATTTTATGAGA | 2 |
| POI 1-4 | GOI 1 | Strong-2 | Bonus Ala | CCCCCGCCATGGCG | 86 |
| POI 1-5 | GOI 1 | Adequate | MASS₁ | CTTACCACCATGGCGTCCTCC | 89 |
| POI 1-6 | GOI 1 | Adequate | MASS₂ | CTTACCACCATGGCTTCCTCC | 90 |
| POI 1-7 | GOI 1 | Adequate | MAA | CTTACCACCATGGCGGCC | 91 |
| POI 1-8 | GOI 1 | Adequate | MASL | CTTACCACCATGGCGTCCCTC | 92 |
| POI 1-9 | GOI 1 | Adequate | MAAL | CTTACCACCATGGCGGCCCTC | 93 |
| POI 1-10 | GOI 1 | Strong-1 | MASS | GCGGCAGCCATGGCGTCCTCC | 94 |
| POI 2-1* | GOI 2 | Strong-3 | Original | GTGACCGCCATGGAC | 95 |
| POI 2-2 | GOI 2 | Strong-1 | Bonus Ala | GCGGCAGCCATGGCG | 88 |
| POI 2-3 | GOI 2 | Depleted | Bonus Arg | GTTTATTTTATGAGA | 2 |
| POI 2-4 | GOI 2 | Strong-2 | Bonus Ala | CCCCCGCCATGGCG | 86 |
| POI 2-5 | GOI 2 | Strong-3 | MASS₁ | GTGACCGCCATGGCGTCCTCC | 96 |
| POI 2-6 | GOI 2 | Strong-3 | MAA | GTGACCGCCATGGCGGCC | 97 |
| POI 2-7 | GOI 2 | Strong-3 | MASL | GTGACCGCCATGGCGTCCCTC | 98 |
| POI 2-8 | GOI 2 | Strong-3 | MAAL | GTGACCGCCATGGCGGCCCTC | 99 |
| POI 2-9 | GOI 2 | Strong-1 | MASS1 | GCGGCAGCCATGGCGTCCTCC | 94 |
| POI 3-1* | GOI 3 | Adequate | Original | GGTACCGCCATGACT | 100 |
| POI 3-2 | GOI 3 | Strong-1 | Bonus Ala | GCGGCAGCCATGGCG | 88 |
| POI 3-3 | GOI 3 | Depleted | Bonus Arg | GTTTATTTTATGAGA | 2 |
| POI 3-4 | GOI 3 | Strong-2 | Bonus Ala | CCCCCGCCATGGCG | 86 |
| POI 3-5 | GOI 3 | Adequate | MASS₁ | GGTACCGCCATGGCGTCCTCC | 101 |
| POI 3-6 | GOI 3 | Adequate | MAA | GGTACCGCCATGGCGGCC | 102 |
| POI 3-7 | GOI 3 | Adequate | MASL | GGTACCGCCATGGCGTCCCTC | 103 |
| POI 3-8 | GOI 3 | Adequate | MAAL | GGTACCGCCATGGCGGCCCTC | 104 |
| POI 3-9 | GOI 3 | Strong-1 | MASS₁ | GCGGCAGCCATGGCGTCCTCC | 94 |
| POI 4-1* | GOI 4 | Strong-4 | Original | GTCGCCGCCATGGCG | 105 |
| POI 4-2 | GOI 4 | Strong-1 | Original | GTGCGGCAGCCATGGCG | 106 |
| POI 4-3 | GOI 4 | Depleted | Bonus Arg | GTTTATTTTATGAGA | 2 |
| POI 4-4 | GOI 4 | Strong-2 | Original | GTCCCCCGCCATGGCG | 107 |
| POI 4-5 | GOI 4 | Strong-4 | MASS₁ | GTCGCCGCCATGGCGTCCTCC | 108 |
| POI 4-6 | GOI 4 | Strong-4 | MAA | GTCGCCGCCATGGCGGCC | 109 |
| POI 4-7 | GOI 4 | Strong-4 | MASL | GTCGCCGCCATGGCGTCCCTC | 110 |
| POI 4-8 | GOI 4 | Strong-4 | MAAL | GTCGCCGCCATGGCGGCCCTC | 111 |
| POI 4-9 | GOI 4 | Strong-1 | MASS₁ | GTGCGGCAGCCATGGCGTCCTCC | 112 |

Protoplast transformation: Maize leaf protoplasts were isolated from etiolated seedlings as described by Sheen and Bogorad, 1985. Protoplasts were transformed with the constructs described in Table 5 using PEG mediated transformation (Yoo et al., 2007, Nature Protocols., 2, 1565-1572). A luciferase expression construct was co-transformed and served as a transformation control. Protoplasts were incubated 18 to 24 hours at 22° C. Twenty-four replicates were performed for each treatment. In each replicate, 54k protoplasts were transformed. Twenty-four replicates were pooled into four replicates for each treatment. Aliquots equal to 258k cells and 54k cells were removed and processed for protein quantification and RNA quantification, respectively. The remaining of protoplasts were used for luciferase quality control and normalization assays.

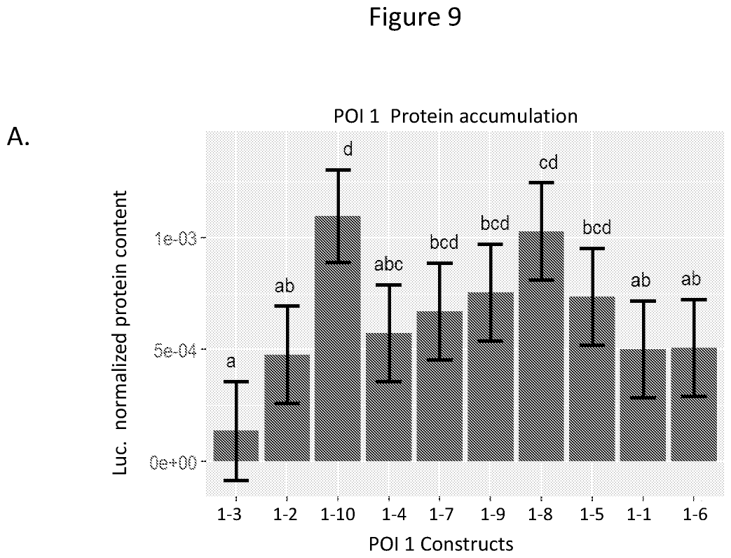

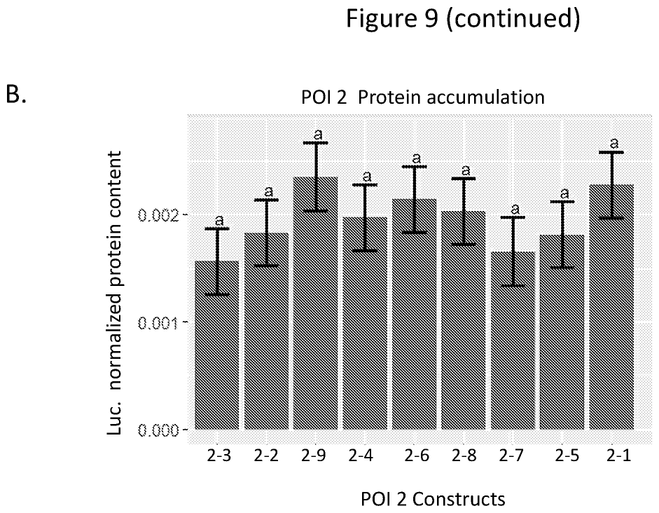

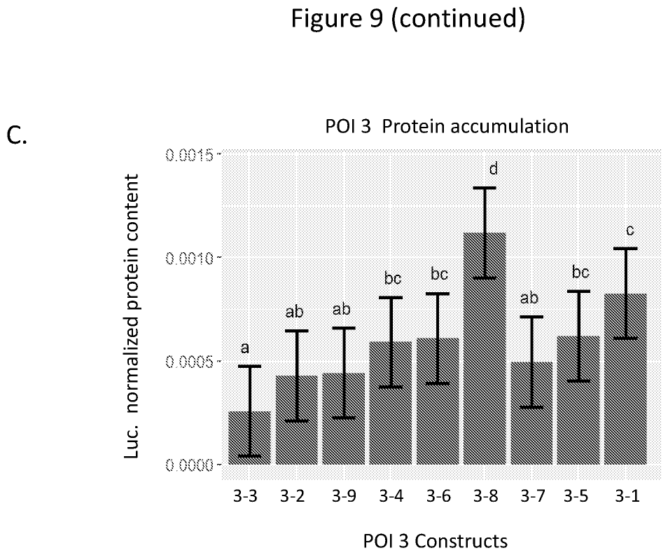

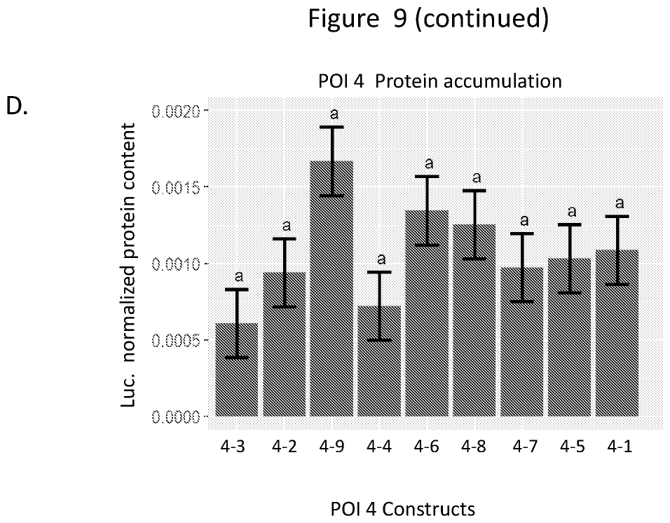

Protein extraction and quantitation: Protein was extracted from maize leaf protoplast samples via phosphate-buffered saline with Tween detergent. Proteins of interest were quantitated via ELISA (enzyme-linked immunosorbent assay) with internally-developed antibodies (FIG. 9). Proteins of interest were normalized to total proteins via BCA Total Protein assay (Pierce, ThermoFisher, Carlsbad, Calif.). For protoplasts, proteins of interest were also normalized to co-transformed luciferase levels.

RNA extraction, purification: Two stainless steel BBs were added to each protoplast well on a 96 well plate along with 200 μL TRI reagent. Cells were homogenized at 1100-1200 rpm for 4 min. RNA was extracted and purified using TM reagent (Sigma) and Direct-zol (Zymo) 96 well kits, according to manufacturers' instructions. After elution into RNase-free water, Turbo DNase (ThermoFisher, Carlsbad, Calif.) digestion was performed according to the manufacturer's instructions.

RNA quantitation: MultiScribe Reverse Transciptase (ThermoFisher, Carlsbad, Calif.) was used to generate cDNA with the following reaction conditions: 25° C. for 10 minutes, 37° C. for 2 hours, 85° C. for 5 minutes, 4° C. hold. TaqMan quantitative PCR was performed with PerfeCTa FastMix II 2X (Quantabio, Beverly, Mass.). Reactions were denatured at 95° C. for 2 minutes, and then cycled 40× with: 95° C. for 10 seconds, 60° C. for 30 seconds, and a plate scan.

Impact of Kozak and N-terminal modification on protoplast expression: Kozak and N-terminal modifications can, in maize leaf protoplasts, have a statistically significant effect on protein accumulation, but the effect depends on the context from the gene of interest (FIG. 9). Specifically, there were strong and significant differences in protein accumulation for POI 1 and POI 3 due to Kozak/N-terminal modifications, but the ranking of Kozak/N-terminal modifications is not the same between POI 1 and POI 3. For example, the highest protein accumulation for POI 3 was from the MAAL N-terminal modification in the context of an unoptimized Kozak sequence (see FIG. 9d). Whereas for POI 1, the highest protein accumulation was from a modified strong Kozak sequence and a MASS N-terminal modification (see FIG. 9a). The protein accumulation differences between specific constructs are large, on the order of 5 to 10 fold. Not wishing to be bound by a particular theory, these large effects may be due to improved ribosomal recruitment and translation initiation and/or enhancement (see Kozak, J. of Biol Chem., 1991, 266, 19867-19870). Constructs with the depleted Kozak sequence consistently showed lower protein expression. For POI 1 and POI 3, this decrease was statistically significant.

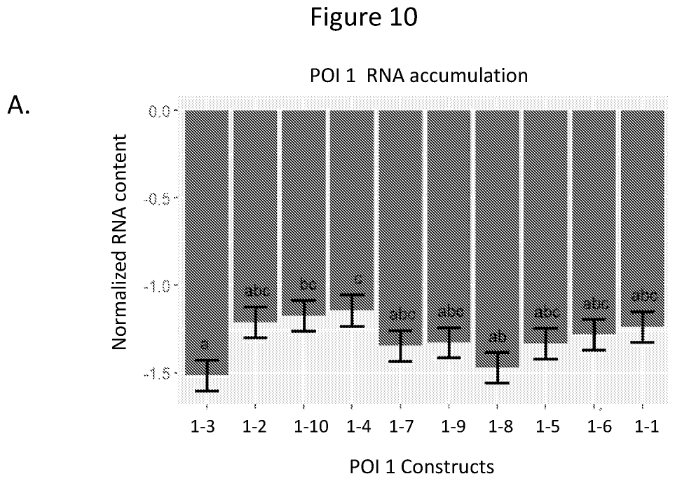

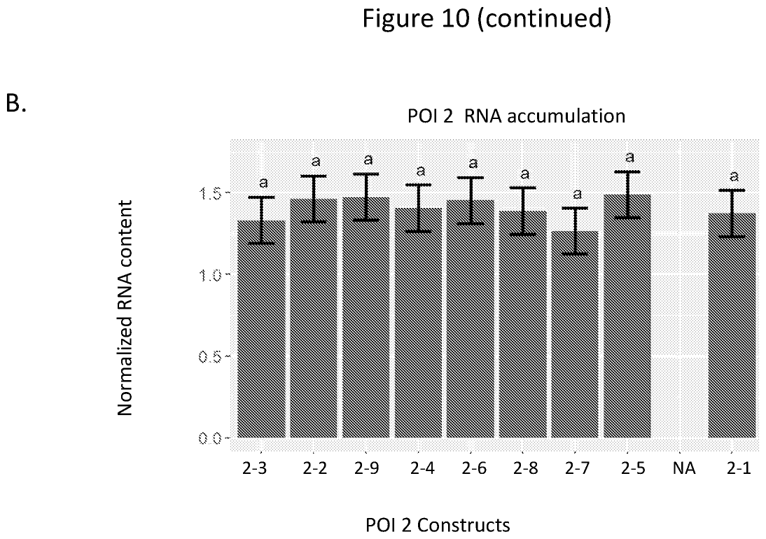

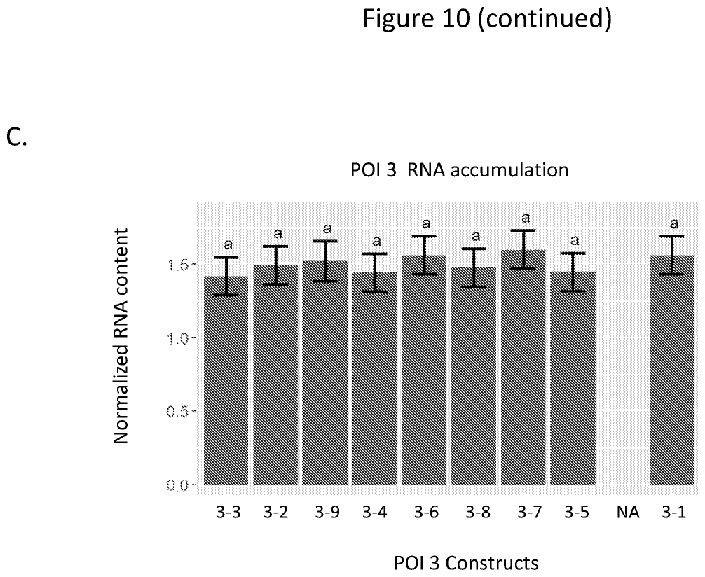

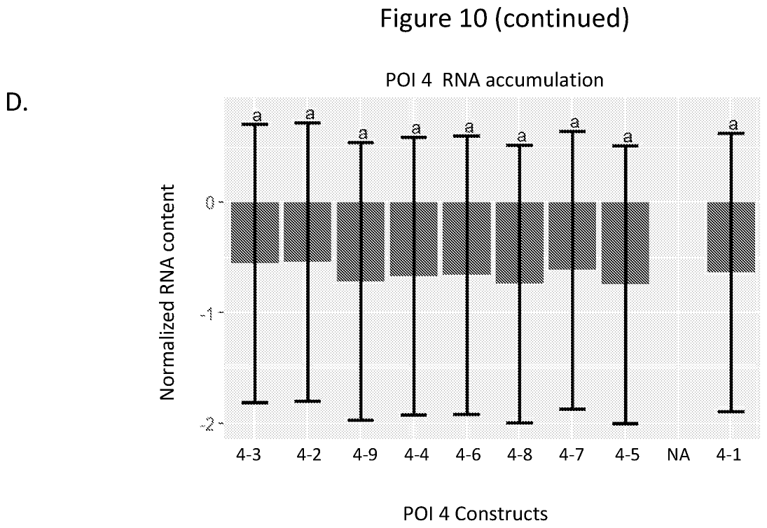

Kozak and N-terminal modifications did not have significant effects at the RNA level for POI 2, 3 and 4 (FIG. 10). POI 1 constructs (FIG. 10a) showed significant differences in RNA accumulation, but effects were small and did not match the effect on Protein accumulation in FIG. 9a. For example, the highest POI 1 protein accumulation was from strong Kozak with MASS N-terminal modification and from Original Kozak with MASL modification, but these same constructs do not cause the highest RNA accumulation. The RNA accumulation differences between constructs were small, less than 1.5 fold. Not wishing to be bound by a particular theory, the small effects on RNA accumulation observed may be due to changes in ribosomal recruitment causing changes in mRNA stability (Presnyak et al., 2015, Cell, 160, 1111-1124).

Overall, these results are consistent with Kozak and N-terminal modifications effecting transgene expression at the protein accumulation level in a context-dependent fashion, while gene expression at the RNA level is unchanged or changed only slightly by these same modifications.

TABLE 6
* Indicates the constructs comprising unoptimized Kozak sequence with original N-terminal
sequence for the specified gene.
Mean protein accumulation and percent difference compared to transgene
constructs with native Kozak and N-terminal sequences.

| | | | | % difference from |
|---|---|---|---|---|
| | | | | native Kozak with |
| Expression | Kozak | N-terminal | Mean Protein | Original N- |
| construct | Modification | modification | Accumulation | terminal sequence |
| POI 1-1* | Adequate | Original | 5.02E−04 | 0% |
| POI 1-2 | Strong-1 | Bonus Ala | 4.78E−04 | −5% |
| POI 1-3 | Depleted | Bonus Arg | 1.11E−04 | −78% |
| POI 1-4 | Strong-2 | Bonus Ala | 5.74E−04 | 14% |
| POI 1-5 | Adequate | MASS1 | 7.37E−04 | 47% |
| POI 1-6 | Adequate | MASS2 | 5.08E−04 | 1% |
| POI 1-7 | Adequate | MAA | 6.71E−04 | 34% |
| POI 1-8 | Adequate | MASL | 1.03E−03 | 105% |
| POI 1-9 | Adequate | MAAL | 7.55E−04 | 50% |
| POI 1-10 | Strong-1 | MASS | 1.04E−03 | 106% |
| POI 2-1* | Strong-3 | Original | 2.28E−03 | 0% |
| POI 2-2 | Strong-1 | Bonus Ala | 1.83E−03 | −20% |
| POI 2-3 | Depleted | Bonus Arg | 1.57E−03 | −31% |
| POI 2-4 | Strong-2 | Bonus Ala | 1.97E−03 | −13% |
| POI 2-5 | Strong-3 | MASS1 | 1.81E−03 | −20% |
| POI 2-6 | Strong-3 | MAA | 2.14E−03 | −6% |
| POI 2-7 | Strong-3 | MASL | 1.69E−03 | −26% |
| POI 2-8 | Strong-3 | MAAL | 2.03E−03 | −11% |
| POI 2-9 | Strong-1 | MASS1 | 2.38E−03 | 4% |
| POI 3-1* | Adequate | Original | 8.26E−04 | 0% |
| POI 3-2 | Strong-1 | Bonus Ala | 4.29E−04 | −48% |
| POI 3-3 | Depleted | Bonus Arg | 2.58E−04 | −69% |
| POI 3-4 | Strong-2 | Bonus Ala | 5.91E−04 | −28% |
| POI 3-5 | Adequate | MASS1 | 6.21E−04 | −25% |
| POI 3-6 | Adequate | MAA | 6.10E−04 | −26% |
| POI 3-7 | Adequate | MASL | 4.95E−04 | −40% |
| POI 3-8 | Adequate | MAAL | 1.12E−03 | 35% |
| POI 3-9 | Strong-1 | MASS1 | 4.43E−04 | −46% |
| POI 4-1* | Strong-4 | Original | 1.09E−03 | 0% |
| POI 4-2 | Strong-1 | Original | 9.39E−04 | −13% |
| POI 4-3 | Depleted | Bonus Arg | 6.08E−04 | −44% |
| POI 4-4 | Strong-2 | Original | 7.20E−04 | −34% |
| POI 4-5 | Strong-4 | MASS1 | 1.03E−03 | −5% |
| POI 4-6 | Strong-4 | MAA | 1.35E−03 | 24% |
| POI 4-7 | Strong-4 | MASL | 9.74E−04 | −10% |
| POI 4-8 | Strong-4 | MAAL | 1.25E−03 | 16% |
| POI 4-9 | Strong-1 | MASS1 | 1.67E−03 | 54% |

Impact of Kozak and N-terminal modification on in-planta expression: Based on the results from the protoplast assays, the modifications showing the strongest effects were moved into stable transformation testing in maize. Specifically, GOI 1/POI 1 and GOI 3/POI 3 variants were advanced for in planta testing. Table 7 describes the specific constructs that were tested. Agrobacterium mediated transformation was used to transform maize explants with one of the T-DNA constructs described in Table 7. Plants with a single copy of the transgene were outcrossed to non-transgenic plants to generate F1 plants and leaf punches were sampled for expression quantification. Protein and RNA quantification was carried out as described previously for protoplast analysis.

TABLE 7
In planta stable protein expression. Mean protein accumulation and percent
difference from native protein sequence. * Indicates the constructs comprising
unoptimized Kozak sequence with original N-terminal sequence for the specified gene.

| | | | | | % difference |
|---|---|---|---|---|---|
| | | | | | from native |
| | | | | Mean | Kozak with |
| | | | | Protein | Original N- |
| Expression | Gene of | Kozak | N-terminal | Accumulation | terminal |
| Construct | Interest | Modification | Modification | (ppm) | sequence |
| POI 1-1* | GOI 1 | Adequate | Original | 0.90 | 0% |
| POI 1-3 | GOI 1 | Depleted | Bonus Arg | 0.41 | −55% |
| POI 1-8 | GOI 1 | Adequate | MASL | 18.65 | 1973% |
| POI 1-10 | GOI 1 | Strong-1 | MASS | 17.67 | 1863% |
| POI 3-1* | GOI 3 | Adequate | Original | 39.71 | 0% |
| POI 3-3 | GOI 3 | Depleted | Bonus Arg | 2.96 | −93% |
| POI 3-8 | GOI 3 | Adequate | MAAL | 75.29 | 90% |

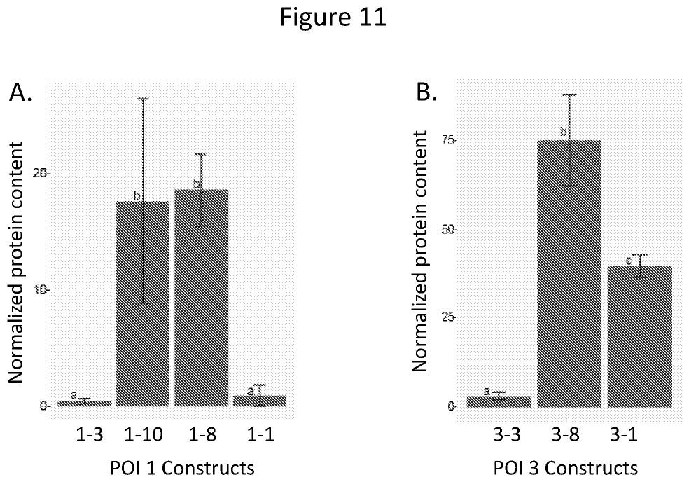

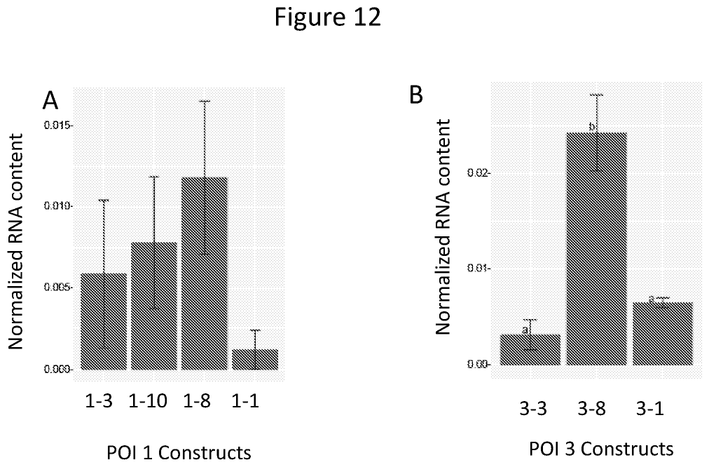

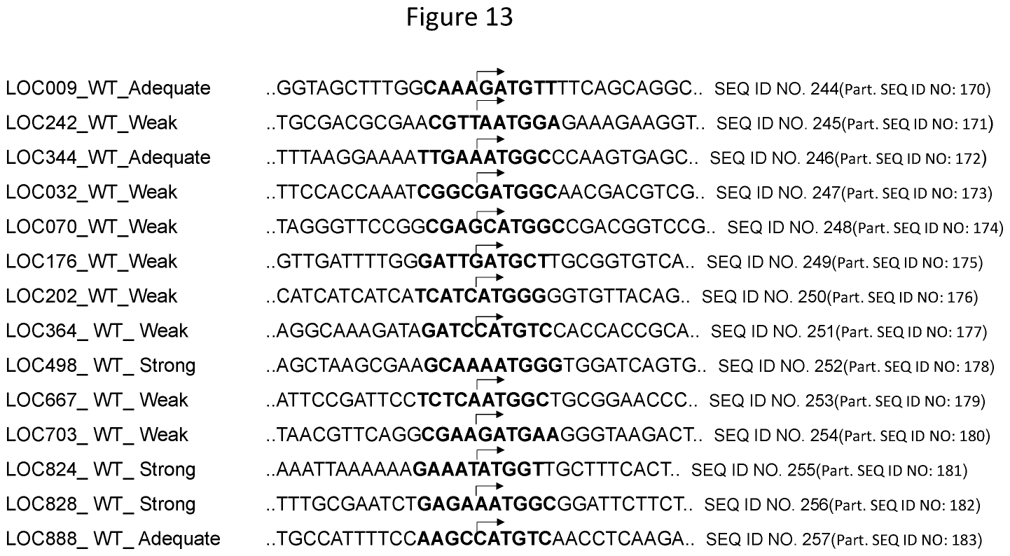

As shown in FIG. 11, the results from stable transformed plants were consistent with observations seen in protoplast assays. For example, for POI 1, the variant with a modified strong Kozak sequence with a MASS N-terminal modification and the adequate Kozak with the MASL N-terminal modification showed significant increase in protein accumulation compared to the adequate Kozak with the original N terminus (ANOVA F=10.2, p=0.000378) (see FIG. 11A and Table 7). For POI 3, significant differences in protein accumulation across variants was also observed (ANOVA F=25.01, p=0.00000476). See FIG. 11B and Table 7. The adequate Kozak with the MAAL modification showed the highest protein accumulation. For both proteins, the depleted Kozak sequence resulted in statistically significant reduction in protein accumulation. Significant changes in RNA expression were not observed for GOI 1, but were noted for GOI 3 (see FIG. 12).

Taken together, the data suggests that Kozak and N-terminal modifications can affect transgene protein accumulation in protoplasts and stable corn transformants.

### Example 11: Additional Soy Target Genes

Thirteen soy genes with a range of Kozak sequence strengths are chosen to test the effect of targeted manipulations of Kozak sequences on protein expression levels. The strength of the native Kozak sequence was determined as described in Example 1 by comparing the sequence features of the native Kozak sequence to a consensus sequence derived aligning the Kozak sequences of the top 100 Arabidopsis genes exhibiting high mRNA expression and ribosomal protection. The genomic regions surrounding the Kozak sequences of these genes, and their predicted ability to drive high translational efficiency (strong, adequate, weak) are shown Table 8. Genomic sequence around the Kozak sites of the 13 genes was analyzed to identify Cas12a CRISPR targets sites (see Table 9).

TABLE 8
Soy Target genes. The SEQ ID NOs represent genomic fragments of the target
gene comprising the Kozak sequence, region of
the 5′UTR and region of exon 1 comprising the start site.

| | | | Predicted | |
|---|---|---|---|---|
| | | | strength of | |
| | Gene Name | | the native | SEQ ID |
| Name | (GenBank) | Description | Kozak | NO |
| LOC009 | LOC114375009 | Gm seed linoleate 13S- | adequate | 170 |
| | | lipoxygenase-1 | | |
| LOC242 | LOC114377242 | Gm centromere protein C-like, | adequate | 171 |
| | | transcript variant X2 | | |
| LOC344 | LOC114417344 | Gm 3-phosphoshikimate 1- | adequate | 172 |
| | | carboxyvinyltransferase 2 | | |
| LOC032 | LOC100795032 | Gm eukaryotic initiation factor 4A- | weak | 173 |
| | | 3 | | |
| LOC070 | LOC114398070 | Gm nuclear transcription factor Y | adequate | 174 |
| | | subunit B-10-like | | |
| LOC176 | LOC114417176 | Gm transcription activator GLK1- | weak | 175 |
| | | like | | |
| LOC202 | LOC114400202 | Gm protein NUCLEAR FUSION | adequate | 176 |
| | | DEFECTIVE 4-like | | |
| LOC364 | LOC114425364 | Gm MYB-like transcription factor | weak | 177 |
| | | ETC3 | | |
| LOC498 | LOC114375498 | Gm monothiol glutaredoxin-S17 | adequate | 178 |
| LOC667 | LOC114373667 | Gm lactoylglutathione lyase | adequate | 179 |
| LOC703 | LOC102667703 | Gm B-box zinc finger protein 32 | adequate | 180 |
| LOC824 | LOC114369824 | Gm protein leghemoglobin A | adequate | 181 |
| LOC828 | LOC114423828 | Gm 14-3-3-like protein A | strong | 182 |
| LOC888 | LOC114386888 | Gmethylene-responsive | adequate | 183 |
| | | transcription factor ERF086-like | | |

TABLE 9
List of representative Caslla CRISPR target sites at or near
the Kozak sequences of soy genes

| | | | Target site sequence | | |
|---|---|---|---|---|---|
| Gene | Enzyme | Target site name | PAM | Spacer | SEQ ID NO |
| LOC009 | FnCas12a | LOC009_FnCas12a_TS1 | TTG | GCAAAGATGTTTTCAGCAGGCCA | 184 |
| | FnCas12a | LOC009_FnCas12a_TS2 | TTG | CCAAAGCTACCAACACAACTATT | 185 |
| | FnCas12a | LOC009_FnCas12a_TS3 | TTG | GTAGCTTTGGCAAAGATGTTTTC | 186 |
| | FnCas12a | LOC009_FnCas12a_TS4 | TTG | TGTTGGTAGCTTTGGCAAAGATG | 187 |
| | LbCas12a | LOC009_LbCas12a_TS1 | TTTG | GCAAAGATGTTTTCAGCAGGCCA | 188 |
| | LbCas12a | LOC009_LbCas12a_TS2 | TTTG | CCAAAGCTACCAACACAACTATT | 189 |
| | LbCas12a | LOC009_LbCas12a_TS3 | TTTG | ATCTTATGGCCTGCTGAAAACAT | 190 |
| | LbCas12a-RR | LOC009_LbCas12a-RR_TS1 | TCCC | TTTGATCTTATGGCCTGCTGAAA | 191 |
| LOC242 | FnCas12a | LOC242_FnCas12a_TS1 | TTG | TCCATTAACGTTCGCGTCGCATT | 192 |
| | FnCas12a | LOC242_FnCas12a_TS2 | TTG | CGAACCAATAATGCGACGCGAAC | 193 |
| | FnCas12a | LOC242_FnCas12a_TS3 | TTG | TTTCTCCATTAACGTTCGCGTCG | 194 |
| | FnCas12a | LOC242_FnCas12a_TS4 | TTA | ACGTTCGCGTCGCATTATTGGTT | 195 |
| | FnCas12a | LOC242_FnCas12a_TS5 | TTA | TCTATTTCCGAACCAATAATGCG | 196 |
| | LbCas12a | LOC242_LbCas12a_TS1 | TTTG | TCCATTAACGTTCGCGTCGCATT | 197 |
| | LbCas12a | LOC242_LbCas12a_TS2 | TTTG | CGAACCAATAATGCGACGCGAAC | 198 |
| | LbCas12a-RR | LOC242_LbCas12a-RR_TS1 | TCCA | CTAATGCATCACCTTCTTTCTCC | 199 |
| | LbCas12a-RR | LOC242_LbCas12a-RR_TS2 | TCCA | TTAACGTTCGCGTCGCATTATTG | 200 |
| | LbCas12a-RR | LOC242_LbCas12a-RR_TS3 | TCCG | AACCAATAATGCGACGCGAACGT | 201 |
| LOC344 | FnCas12a | LOC344_FnCas12a_TS1 | TTA | AGGAAAATTGAAATGGCCCAAGT | 202 |
| | FnCas12a | LOC344_FnCas12a_TS2 | TTG | AGCAAGATTGTGCACTCTGCTCA | 203 |
| | FnCas12a | LOC344_FnCas12a_TS3 | TTG | ACAACTTTAAGGAAAATTGAAAT | 204 |
| | FnCas12a | LOC344_FnCas12a_TS4 | TTG | GGCCATTTCAATTTTCCTTAAAG | 205 |
| | FnCas12a | LOC344_FnCas12a_TS5 | TTG | TGCACTCTGCTCACTTGGGCCAT | 206 |
| | LbCas12a | LOC344_LbCas12a_TS1 | TTTA | AGGAAAATTGAAATGGCCCAAGT | 207 |
| | LbCas12a | LOC344_LbCas12a_TS2 | TTTG | AGCAAGATTGTGCACTCTGCTCA | 208 |
| | LbCas12a-RR | LOC344_LbCas12a-RR_TS1 | TTCA | CAACTTTAAGGAAAATTGAAATG | 209 |
| LOC667 | FnCas12a | LOC667_FnCas12a_TS1 | TTG | CGATTCCTCTCAATGGCTGCGGA | 210 |
| | FnCas12a | LOC667_FnCas12a_TS2 | TTG | CTCTCAATGGCTGCGGAACCCAA | 211 |
| | FnCas12a | LOC667_FnCas12a_TS3 | TTG | CGCAGCCATTGAGAGGAATCGGA | 212 |
| | FnCas12a | LOC667_FnCas12a_TS4 | TTG | CTTGGGTTCCGCAGCCATTGAGA | 213 |
| | LbCas12a-RR | LOC667_LbCas12a-RR_TS1 | TTCC | GATTCCTCTCAATGGCTGCGGAA | 214 |
| | LbCas12a-RR | LOC667_LbCas12a-RR_TS2 | TTCC | TCTCAATGGCTGCGGAACCCAAG | 215 |
| | LbCas12a-RR | LOC667_LbCas12a-RR_TS3 | TTCC | GCAGCCATTGAGAGGAATCGGAA | 216 |
| | LbCas12a-RR | LOC667_LbCas12a-RR_TS4 | TTCC | TTGGGTTCCGCAGCCATTGAGAG | 217 |
| LOC070 | FnCas12a | LOC070_FnCas12a_TS2 | TTC | CCTTTCTCAAATTAGGGTTCCGG | 218 |
| | FnCas12a | LOC070_FnCas12a_TS3 | TTC | CGGCGAGCATGGCCGACGGTCCG | 219 |
| | LbCas12a-RR | LOC070_LbCas12a-RR_TS2 | TTCC | CTTTCTCAAATTAGGGTTCCGGC | 220 |
| | LbCas12a-RR | LOC070_LbCas12a-RR_TS3 | TTCC | GGCGAGCATGGCCGACGGTCCGG | 221 |
| LOC824 | FnCas12a | LOC824_FnCas12a_TS1 | TTC | TCAGTGAAAGCAACCATATTTCT | 222 |
| LOC498 | FnCas12a | LOC498_FnCas12a_TS1 | TTC | ACGTCCCTCACTGATCCACCCAT | 223 |
| | LbCas12a-RR | LOC498_LbCas12a-RR_TS1 | TTCA | CGTCCCTCACTGATCCACCCATT | 224 |
| LOC703 | FnCas12a | LOC703_FnCas12a_TS1 | TTC | AGGCGAAGATGAAGGGTAAGACT | 225 |
| | LbCas12a-RR | LOC703_LbCas12a-RR_TS1 | TTCA | GGCGAAGATGAAGGGTAAGACTT | 226 |
| LOC888 | FnCas12a | LOC888_FnCas12a_TS1 | TTC | TTGCCATTTTCCAAGCCATGTCA | 227 |
| | FnCas12a | LOC888_FnCas12a_TS2 | TTC | TTGAGGTTGACATGGCTTGGAAA | 228 |
| | LbCas12a-RR | LOC888_LbCas12a-RR_TS1 | TTCT | TGCCATTTTCCAAGCCATGTCAA | 229 |
| | LbCas12a-RR | LOC888_LbCas12a-RR_TS2 | TTCT | TGAGGTTGACATGGCTTGGAAAA | 230 |
| LOC202 | FnCas12a | LOC202_FnCas12a_TS3 | TTC | TCCTGTAACACCCCCATGATGAT | 231 |
| LOC828 | FnCas12a | LOC828_FnCas12a_TS1 | TTG | CGAATCTGAGAAATGGCGGATTC | 232 |
| | LbCas12a | LOC828_LbCas12a_TS1 | TTTG | CGAATCTGAGAAATGGCGGATTC | 233 |
| | FnCas12a | LOC828_FnCas12a_TS2 | TTG | TAGTTGCGGTGGTGGACATGGAT | 234 |
| LOC032 | FnCas12a | LOC032_FnCas12a_TS2 | TTC | AAACCTTTTTTTTTCCACCAAAT | 235 |
| | FnCas12a | LOC032_FnCas12a_TS3 | TTC | CACCAAATCGGCGATGGCAACGA | 236 |
| | LbCas12a | LOC032_LbCas12a_TS2 | TTTC | AAACCTTTTTTTTTCCACCAAAT | 237 |
| | LbCas12a | LOC032_LbCas12a_TS3 | TTTC | CACCAAATCGGCGATGGCAACGA | 238 |
| LOC176 | FnCas12a | LOC176_FnCas12a_TS2 | TTA | GATTAACATAGTGTGTTGATTTT | 239 |
| | FnCas12a | LOC176_FnCas12a_TS3 | TTG | GGATTGATGCTTGCGGTGTCACC | 240 |
| | LbCas12a | LOC176_LbCas12a_TS2 | TTTA | GATTAACATAGTGTGTTGATTTT | 241 |
| | LbCas12a | LOC176_LbCas12a_TS3 | TTTG | GGATTGATGCTTGCGGTGTCACC | 242 |

### Example 12: Evaluating the Efficacy CRISPR Mediated Chromosome Cutting

The LOC 344 gene was chosen for further analysis. Cas12a guide RNA expression cassettes were designed to guide LbCas12a, or FnCas12a to appropriate target sites at or around the Kozak sequence identified within the LOC 344 gene (see Table 9). The gRNA cassettes comprised a soy U6 Pol III promoter operably linked to a CRISPR direct repeat for either FnCas12a (SEQ ID NO:70) or LbCas12a (SEQ ID NO: 169) operably linked to a 23- to 25-nucleotide spacer DNA sequence targeting a site within LOC 344 (SEQ ID NO: 202-209) and a polyT (TTTTTTTT) transcription terminator sequence. The gRNA cassettes were inserted into a pUC57 variant of the pUC19 vector (Yanisch-Perron et al., 1985).

Transient Soy protoplast assays were used to test for guide RNA efficacy. The guide RNA vectors were co-transformed via polyethylene-glycol (PEG) into soy cotyledon protoplasts with another binary vector encoding the appropriate FnCas12a or LbCas12a CRISPR endonuclease.

TABLE 10
Combination of reagents used for protoplast gRNA efficacy assay.

| | Target | | |
|---|---|---|---|
| Treatment | gene | Target site gRNA | Enzyme |
| 1 | LOC 344 | LOC734_FnCas12a_TS1 | FnCas12a |
| 2 | | LOC344_FnCas12a_TS2 | FnCas12a |
| 3 | | LOC344_FnCas12a_TS3 | FnCas12a |
| 4 | | LOC344_FnCas12a_TS4 | FnCas12a |
| 5 | | LOC344_FnCas12a_TS5 | FnCas12a |
| 6 | | LOC344_LbCas12a_TS1 | FnCas12a |
| 7 | | LOC344_LbCas12a_TS2 | FnCas12a |

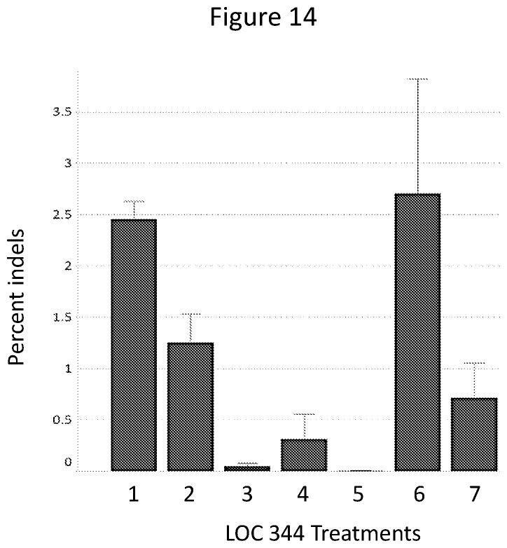

After a two-day incubation period, genomic DNA was isolated from protoplast suspensions and target regions were amplified by PCR (9 cycles of touchdown PCR from 67 to 58° C. annealing followed by 30 cycles of standard PCR with 58° C. annealing). The amplicons were sequenced by Next Generation Sequencing (NGS), by standard methods known in the art to identify modified sequences comprising insertions or deletions (indels) that are indicative of guide RNA-Cas12a mediated editing. The gRNA efficacy data is shown in FIG. 14. For LOC 344, cutting TS1 with FnCas12a or LbCas12a resulted in the highest editing efficiency.

### Example 13: Editing Kozak Sequences in Soy Protoplasts

Based on the gRNA efficacy data for LOC 344, the highest cutting gRNA nuclease combinations were selected for testing templated editing at the Kozak target sites. As shown in Table 8, the native LOC 344 Kozak sequence (nucleotides −9 to +12 flanking the translation initiator codon (ATG) of SEQ ID NO: 258) was determined to be an adequate Kozak based on comparison to a consensus sequence derived from aligning the Kozak sequences of 100 Arabidopsis genes exhibiting high mRNA expression and ribosomal protection. Editing systems comprising gRNAs targeting TS1 and cognate Cas endonucleases, FnCas12a protein (SEQ ID NO: 261) and LbCas12a protein (SEQ ID NO: 262), were assembled in vitro as ribonucleoprotein (RNP) complexes along with single stranded DNA repair (donor) template. The repair DNA template for LOC 344 (SEQ ID NO: 243) comprised an engineered strong Kozak consensus sequence flanked by homology arms that were homologous to the genic sequence flanking the native Kozak sequence. The single stranded repair DNA template was phosphorothioated at the last two phosphodiester bonds of each termini to make it resistant to nuclease degradation (Renaud et al., 2016). Protoplasts were transformed with various assay combinations are shown in Table 11 by standard PEG mediated transformation method known in the art.

TABLE 11
Combination of reagents used for LOC 344 templated editing assay.

| | | | Repair |
|---|---|---|---|
| | | | template |
| Treatment | Target site gRNA | Enzyme | orientation |
| 1 | LOC344_LbCas12a_TS1 | LbCas12a | Sense |
| 2 | LOC344_LbCas12a_TS1 | LbCas12a | Antisense |
| 3 | LOC344_FnCas12a_TS1 | FnCas12a | Sense |
| 4 | LOC344_FnCas12a_TS1 | FnCas12a | Antisense |
| 5 (control) | — | — | Sense |
| 6 (control) | — | — | Antisense |

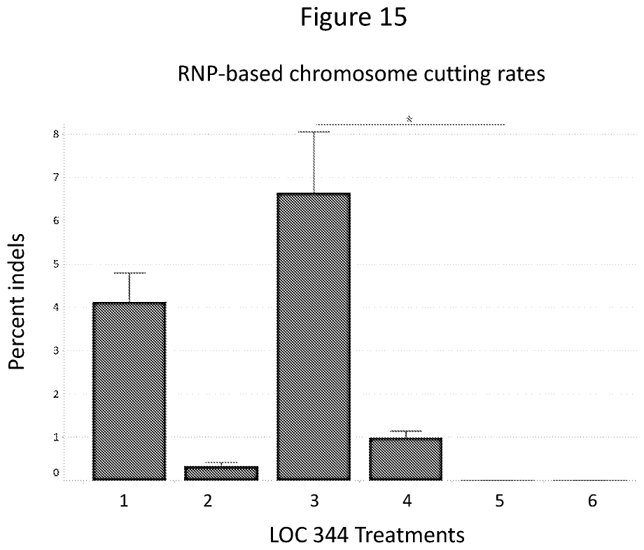

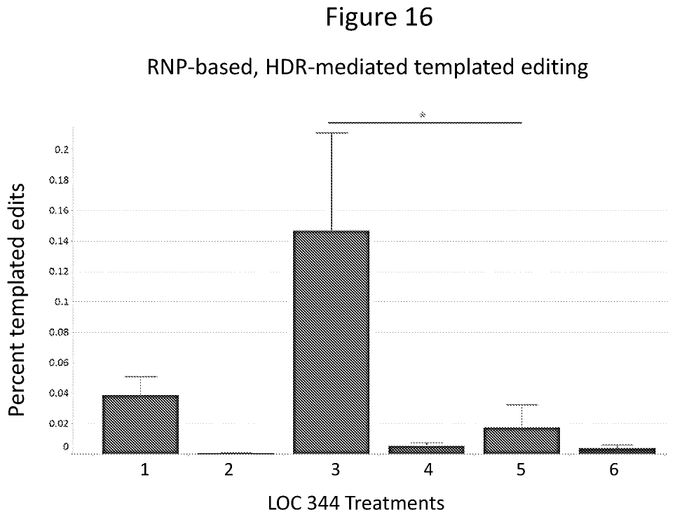

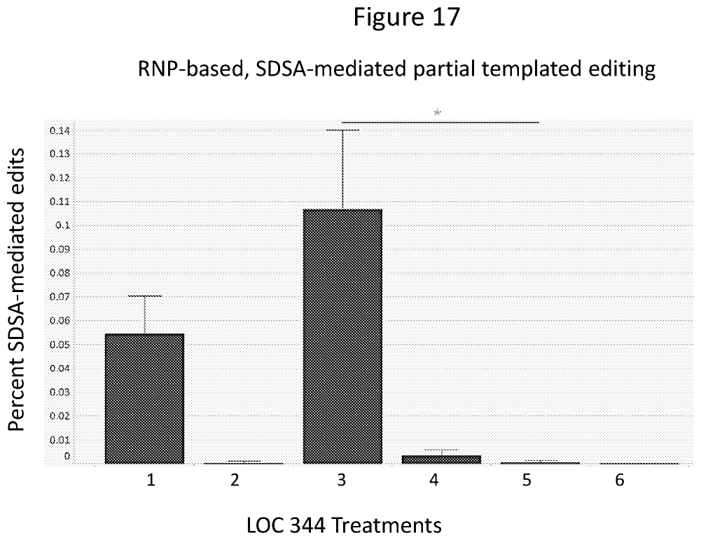

After a two-day incubation period, genomic DNA was isolated from protoplast suspensions and target regions were amplified by PCR. The amplicons were sequenced by Next Generation Sequencing (NGS), by standard methods known in the art to assay for presence of edits and identify targeted integrations of repair template. The RNP based chromosome indel rates (see FIG. 15) as well as templated editing rates (see FIGS. 16 and 17) were quantified for each treatment. At least one RNP/repair template combination demonstrated statistically significant, above-background chromosome cutting and HDR-mediated repair template integration as revealed by quantification of indels and templated edits, respectively (see FIG. 16). Donor integrations that were not mediated by homology upstream of the Kozak sequence, but otherwise demonstrated perfect homology downstream of the Kozak region can also be of value for this analysis. Therefore, this kind of integrations were also quantified and were collectively denoted as SDSA (synthesis-dependent strand-annealing)-mediated integrations. Representative sequences from HDR-mediated and SDSA-mediated integration events are provided as SEQ ID NO: 259 and SEQ ID NO: 260, respectively. Taken together, this data shows that the native Kozak can be replaced with an engineered Kozak sequence using homology directed insertion following Cas12a mediated cleavage. Furthermore, as seen for LOC344, an endogenous adequate Kozak sequence can be replaced with a strong Kozak sequence.

### Example 14: Editing Kozak Sequences in Soy Calli

Soy callus cells will be used to generate desired edits and determine impact on protein and RNA accumulation. The editing components will be delivered as ribonucleoprotein (RNP) complexes that are assembled in vitro, prior to transformation. gRNAs targeting select target sites will be assembled in vitro with their cognate Cas endonucleases, FnCas12a and LbCas12a, respectively. Then ss or ds stranded repair template DNA will be added to the RNP complex in equimolar concentration. The repair template DNA comprises the desired Kozak modification flanked by homology arms. dsDNA comprising an NptII antibiotic resistance cassette is also added to the mixture as selectable marker for kanamycin selection. This RNP/DNA mixture is transformed into soy callus cells using PEG mediated transformation using standard methods known in the art. As controls, cells will be transformed with complexes lacking the guide RNA-Cas endonuclease complex. Callus cells will be induced for cell division, which will ultimately give rise to callus particles.

The calli will be genotyped by sequencing. Control and edited calli will subsequently be assayed for altered ribosome-binding characteristics and changes in protein accumulation will be quantified by at least two approaches: semi-quantitative Western blot and RiboSeq. To accommodate the analyzes listed above, the individual callus particles will be split into at least three segments. Total genomic DNA will be isolated from one segment and the Kozak regions will be sequenced by Next generation Sequencing methods known in the art (e.g., AmpliSeq, Illumina, San Diego, Calif.) and analyzed for targeted edits. Total proteins will be purified from another segment of edited calli. Protein extracts will be subject to semi-quantitative Western blots using specific antibodies that can detect the target proteins. Significantly altered intensities of Western bands will indicate altered protein accumulation. Total RNA and ribosome-protected RNA will be isolated from the third segment of edited callus particles. Ribo-seq will be used to quantify ribosome occupancy on altered Kozak sequences in test and control calli. For ribo-seq analysis, ribosomal footprinting will be performed using a modified version of a published protocol (Ingolia et al., 2012). Specifically, frozen tissue will be ground to powder using liquid nitrogen, a mortar, and a pestle. 100 mg of tissue will be combined with 400 μL pre-chilled polysome extraction buffer (2% polyoxyethylene (10) tridecyl ether, 1% deoxycholic acid, 1 mM DTT, 100 μg/ul cycloheximide, 10 Units/mL DNase I (epicentre), 100 mM Tris-HCl (pH 8), 40 mM KCl, 20 mM MgCl2). RNA will be digested via RNase I (Ambion, Thermo Fisher, Waltham, Mass.). MicroSpin S-400 Columns (Illustra, GE Healthcare, Chicago, Ill.) will be used to clean up reactions as described. The rRNA removal step will be eliminated, and the RNA will be gel purified using 15% polyacrylamide TBE-Urea gels (Invitrogen, Carlsbad Calif.) and a ZR small-RNA ladder (Zymo Research, Irvine, Calif.). RNA will be recovered from gel slices using Ist Engineering Gel Break and 5 μM column tubes before being pelleted as described but using a ten-minute incubation at −80° C. and centrifugation at 15,000 g for 15 minutes. Purified ribosome footprints will be prepared for sequencing using Illumina TruSeq Small RNA Library Preparation Kits Companion RNA-seq libraries are made from the same tissue samples using KAPA RNA HyperPrep kits (Roche, Indianapolis, Ind.). The resulting ribo-seq and RNA-seq libraries are sequenced using an Illumina NextSeq. Ribo seq and RNA seq analysis will be carried out as described in Example 1.

The sufficiency of Kozak edits to change endogenous gene expression will be confirmed in stably edited soy plants. The same CRISPR reagents will be transformed into explants using particle bombardment. Genotyping by Next gen sequencing methods will identify R0 plants with altered Kozak sequences. Edited individuals will be self-pollinated and plants with homozygous Kozak edits will be identified in the R1 generation by genotyping. The phenotyping experiments described above will also be performed in R1 plants.

1. A method of altering protein accumulation in an edited eukaryotic cell, the method comprising editing the Kozak sequence of a nucleic acid molecule encoding the protein at one or more nucleotides of positions −9, −8, −7, −6, −5, −4, −3, −2, −1, +4, and +5 of the Kozak sequence to generate an edited nucleic acid molecule comprising an edited Kozak sequence, wherein the edited eukaryotic cell comprising the edited nucleic acid molecule exhibits a statistically significant alteration of the accumulation of the protein as compared to the accumulation of the protein within a control eukaryotic cell comprising a reference nucleic acid sequence.

2. The method of claim 1, wherein the protein accumulation is increased in the edited eukaryotic cell as compared to the control eukaryotic cell.

3. The method of claim 1, wherein the protein accumulation is decreased in the edited eukaryotic cell as compared to the control eukaryotic cell.

4. The method of claim 1, wherein the edited Kozak sequence comprises a sequence selected from the group consisting of SEQ ID NOs: 1-7, 86-89, 95 and 105.

5. The method of claim 1, wherein the edited Kozak sequence is a depleted Kozak sequence.

6. The method of claim 1, wherein the protein comprises one or more N-terminal amino acid modifications.

7. The method of claim 6, wherein the one or more N-terminal amino acid modifications introduces an N-terminal sequence selected from the group consisting of: Alanine wherein Alanine is coded by the codon GCG; Alanine wherein Alanine is coded by the codon GCT; Arginine; Methionine-Alanine-Serine-Serine wherein Alanine is coded by the codon GCG; Methionine-Alanine-Serine-Serine wherein Alanine is coded by the codon GCT; Methionine-Alanine-Alanine; Methionine-Alanine-Serine-Leucine; and Methionine-Alanine-Alanine-Leucine.

8. The method of claim 1, wherein one or more of: (a) an A or G at the −3 position is edited to a C or T; (b) a G at the +4 position is edited to an A, C, or T; (c) a C at the −1 position is edited to an A, G, or T; (d) a C at the −2 position is edited to an A, G, or T; (e) an A at the −4 position is edited to a G, C, or T; (f) an A at the −3 position is edited to a G, C, or T; (g) an A at the −2 position is edited to a G, C, or T; (h) an A at the −1 position is edited to a G, C, or T; (i) a G at the +4 position is edited to an A, C, or T; and (j) a C at the +5 position is edited to an A, G, or T.

9. The method of claim 1, wherein one or more of: (a) an C or T at the −3 position is edited to a A or G; (b) an A, C, or T at the +4 position is edited to a G; (c) an A, G, or T at the −1 position is edited to a C; (d) an A, G, or T at the −2 position is edited to a C; (e) a G, C, or T at the −4 position is edited to an A; (f) a G, C, or T at the −3 position is edited to an A; (g) a G, C, or T at the −2 position is edited to an A; (h) a G, C, or T at the −1 position is edited to an A; (i) an A, C, or T at the +4 position is edited to a G; and (j) an A, G, or T at the +5 position is edited to a C.

10. A method of generating an edited plant, the method comprising:
(a) providing an editing enzyme, or a nucleic acid molecule encoding the editing enzyme, to a plant cell;
(b) generating an edit in a Kozak sequence of a nucleic acid molecule encoding a protein in the plant cell to generate an edited Kozak sequence, wherein the edit comprises editing the Kozak sequence in one or more nucleotide positions of the Kozak sequence selected from the group consisting of positions −9, −8, −7, −6, −5, −4, −3, −2, −1, +4, and +5; and
(c) regenerating an edited plant from the plant cell, wherein the edited plant comprises the edited Kozak sequence, and wherein accumulation of the protein is altered in the edited plant as compared to a control plant when grown under comparable conditions.

11. The method of claim 10, wherein accumulation of the protein is increased in the edited plant as compared to the control plant.

12. The method of claim 10, wherein accumulation of the protein is decreased in the edited plant as compared to the control plant.

13. The method of claim 10, wherein the plant cell is selected from the group consisting of a corn cell, a soybean cell, a tomato cell, a rice cell, a canola cell, a pepper cell, a wheat cell, a cucumber cell, an onion cell, an oilseed rape cell, and a cotton cell.

14. The method of claim 10, wherein the nucleic acid molecule is an endogenous nucleic acid molecule or the nucleic acid molecule is a transgenic nucleic acid molecule.

15. The method of claim 10, wherein the edited Kozak sequence comprises a sequence selected from the group consisting of SEQ ID NOs: 1-7, 86-89, 95 and 105.

16. The method of claim 10, wherein the method further comprises generating an edit resulting in one or more N-terminal amino acid modifications of the protein.

17. The method of claim 16, wherein the one or more N-terminal amino acid modifications introduces an N-terminal sequence selected from the group consisting of: Alanine wherein Alanine is coded by the codon GCG; Alanine wherein Alanine is coded by the codon GCT; Arginine; Methionine-Alanine-Serine-Serine wherein Alanine is coded by the codon GCG; Methionine-Alanine-Serine-Serine wherein Alanine is coded by the codon GCT; Methionine-Alanine-Alanine; Methionine-Alanine-Serine-Leucine; and Methionine-Alanine-Alanine-Leucine.

18. The method of claim 10, wherein one or more of: (a) an A or G at the −3 position is edited to a C or T; (b) a G at the +4 position is edited to an A, C, or T; (c) a C at the −1 position is edited to an A, G, or T; (d) a C at the −2 position is edited to an A, G, or T; (e) an A at the −4 position is edited to a G, C, or T; (f) an A at the −3 position is edited to a G, C, or T; (g) an A at the −2 position is edited to a G, C, or T; (h) an A at the −1 position is edited to a G, C, or T; (i) a G at the +4 position is edited to an A, C, or T; and (j) a C at the +5 position is edited to an A, G, or T.

19. The method of claim 10, wherein one or more of: (a) an C or T at the −3 position is edited to a A or G; (b) an A, C, or T at the +4 position is edited to a G; (c) an A, G, or T at the −1 position is edited to a C; (d) an A, G, or T at the −2 position is edited to a C; (e) a G, C, or T at the −4 position is edited to an A; (f) a G, C, or T at the −3 position is edited to an A; (g) a G, C, or T at the −2 position is edited to an A; (h) a G, C, or T at the −1 position is edited to an A; (i) an A, C, or T at the +4 position is edited to a G; and (j) an A, G, or T at the +5 position is edited to a C.

20. An edited eukaryotic cell comprising a recombinant Kozak sequence within a nucleic acid molecule encoding a target protein, wherein the recombinant Kozak sequence comprises one or more mutations as compared to a reference sequence in nucleotides at one or more positions independently selected from the group consisting of positions −9, −8, −7, −6, −5, −4, −3, −2, −1, +4, and +5, wherein the edited eukaryotic cell exhibits altered accumulation of the target protein compared to a control eukaryotic cell.

21.-29. (canceled)
